(19) United States
(12) Patent Application Publication
(10) Pub. No.: US 2024/0254287 A1
(43) Pub. Date: Aug. 1, 2024

(54) Thiol-ene-based polymerizable composition

(71) Applicant: Karl Leibinger Medizintechnik GmbH & Co. KG, Muhlheim an der Donau (DE)

(72) Inventors: Patrick STEINBAUER, Zwettl Osterreich (AT); Stefan BAUDIS, Wein Osterreich (AT); Frank REINAUER, Emmingen-Liptingen (DE); Tobias WOLFRAM, Dreieich (DE)

(21) Appl. No.: 18/561,399

(22) PCT Filed: May 18, 2022
(86) PCT No.: PCT/EP2022/063451

§ 371 Date: Nov. 16, 2023

(30) Foreign Application Priority Data
May 18, 2021 (EP) 21174453.7

(51) Int. Cl. C08G 75/045

(52) U.S. Cl. CPC C08G 75/045; C08G 2261/11; C08G 2261/1412; C08G 2261/1432; C08G 2261/145

(57) ABSTRACT

The present invention refers to the use of compounds comprising, per molecule, at least one polymerizable C—C double bond, at least one adhesion-providing moiety, and one at least bivalent hydrocarbon moiety in between as a spacer as primers in a thiolene-based polymerizable composition for improving the adhesion of the composition on a substrate to be coated therewith, wherein the composition further comprises a radical initiator, monomers with at least two polymerizable C—C double bonds per molecule, and polythiols with at least two SH groups per molecule, wherein the primers comprise 5-norbornen-2-yl groups as polymerizable C—C double bonds, characterized in that compounds are used as primers in the polymerizable composition that a) comprise phosphonic acid groups as adhesion-providing moieties; or b) comprise groups selected from phosphonic acid groups and 3,4-dihydroxyphenyl groups as adhesion-providing moieties, and the polymerizable composition is used as bone adhesive; as well as to corresponding thiol-ene-based compositions; corresponding norbornene derivatives suitable for this purpose; and specific synthesized representatives of such norbornene derivatives.

The present invention relates to the use of special primers in polymerizable thiol-ene-based compositions and their technical and medical use, e.g., in dental materials or as materials for osteosynthesis, in particular as bone adhesives. Furthermore, it relates to novel norbornene derivatives and their corresponding use as primers.

### STATE OF THE ART

Despite the, among vertebrate tissues, unique ability of bones to heal by bone regeneration, this process cannot be performed after severe damages or diseases. Severe bone fractures or injuries due to age, traffic accidents, bone tumor resection, etc. are serious orthopedic problems that additionally require surgical intervention to fixate bones and enable adequate healing. Statistically, every other person will experience a bone fracture during their lifetime. However, classic as well as surgical fracture treatment may always entail complications or problems with regard to mechanical stability, so that there is an urgent need for bone and tissue adhesives (Heiss et al., Eur. J. Trauma 32, 141-148 (2006)).

In addition to the advantageous properties of adhesives when fixating bone fractures, particularly compared to metal implants, e.g., broader applicability, more even distribution of forces to the fracture area, and better fixation in spongious bone parts, bone adhesives still show some difficulties resulting from the special requirements for bone adhesives as well as different application areas. On the one hand, the polymerizable compositions of bone adhesives must be non-toxic and biocompatible in order to be suitable for clinical use in trauma and orthopedic surgery. On the other hand, they should not imitate surrounding tissue and be biologically degradable after a predetermined time span, and heat development during polymerization must be minimal. In addition, the adhesive must bind in humid environments, so that adhesive strength in situ is important. Furthermore, easy fabrication, practicability, and usability, minimum shrinkage after curing, storage stability, and efficiency are relevant factors.

One biomimetic adhesive for repairing bones that is commercially available is, for example, OsStic™ by PBC Biomed, consisting of tricalcium phosphate and phosphoserine. Aqueous solutions of these two components with silicate as an additional component can also be used as soft tissue adhesives (see, e.g., WO 2018/060289 A1, US 2020/038545 A1). These injectable bioceramics show higher shear bond strength (SBS) (2.5-4 MPa) than commercial cyanoacrylates (Pujari-Palmer et al., Materials 11(12), 2492 (2018)). Tetranite™ by LaunchPad Medical is a synthetic, injectable, biometric, and resorbable adhesive based on a combination of tetracalcium phosphate and phosphoserine having ostopromotif properties and ahdere to bones or metals (WO 2010/056811 A1). In addition, it has a 7.5 times higher shear bond strength (3±2 MPa) than non-resorbable poly(methylmethacrylate) bone cement (Kirillova et al., Adv. Healthc. Mater. 7(17), 1800467 (2018)).

Due to the inability of biocompatible and biologically degradable matrix materials to bond directly to hard tissues, adhesive formulations often contain adhesion-providing agent or “primer” molecules. These are extremely important for the final adhesive strength to wet bones and must therefore be well integrated into the adhesive matrix. Usually, the primer molecule consists of a polymerizable group (PG) that is capable to react with other monomers of the polymerizable composition and to copolymerize with the matrix material, as well as an adhesion motif (AM) as a further functional group that is responsible for forming primary chemical bonds (covalent or ionic bonds). In common dental restauration materials, the PG of the primer consist of (meth)acrylates, and the AM consists of phosphonic acid. The etching effect of phosphonic acid results in a number of additional effects, such as the purification of residues from dental enamel, an increase of the enamel surface, and the formation of micropores. In addition, the primers contain a spacer group for connecting PG and AM. Based on the length of this groups, certain properties such as volatility, solubility, viscosity, flexibility, swelling, and hydrophilicity can be controlled (Moszner et al., Dent. Mater. 21(10), 895-910 (2005)).

In patent literature, for example, EP 2,229,930 A1 and DE 10 2004 061 924 A1 disclose primer molecules for dental restauration materials. Commercially available dental enamel-dentin self-etching adhesives (SEA) consist of a self-etching adhesion-providing agent, cross-linking monomers, monofunctional co-monomers, and additives. Adhese® Universal (Ivoclar Vivadent) made of a composite material, methacrylate monomers with methacryloyloxydecyl dihydrogen phosphate (MDP) as an adhesion-providing agent, and Scotchbond™ Universal (3M) made of methacrylates, a silane and MDP as a primer are widely used as dental restauration materials. These adhesion-providing agents showed, depending on the composite material, between 25 and 28 MPa (Todd et al., Ivoclar Vivadent, Adhese Universal—Scientific Documentation, Schaan, 2015).

For tissue engineering of bones, however, (meth)acrylates are not preferred because of their high cytotoxicity and their degradation products. An alternative described in WO 2013/052328 A1 is thiol-ene polymerization with vinyl esters. The free-radical thiolene polymerization is of great interest for biomedical applications such as tissue adhesives, hydrogels, and tooth replacement materials. Step-by-step construction of thiolene networks promotes homogeneous structures that are very advantageous for mechanical robustness. Despite several attempts to use free-radical thiol-ene polymerization in adhesives for tissue restauration, the final properties and the performance of these systems have been insufficient until now. Therefore, there is a continued need for adhesives for fixating bones with biomimetic strategies using phosphonates, amines and catechols representing promising basic approaches (Böker et al., Materials 12(23), 3975 (2019)). A lack of mechanical stiffness and adhesion to wet surfaces remain challenges for thiol-ene adhesives to overcome.

WO 2009/029734 A2 describes different compositions of a polymeric bone cement using thiol-ene chemistry (TEC). The use of bone cement is, however, limited to the injection into bone cavities. WO 2011/048077 A2 discloses diverse compositions for treating bone fractures using thiol-ene chemistry (TEC) describing various groups containing C—C double bonds as polymerizable groups PG, which are selected from vinyl, acrylate, methacrylate and allyl groups as well as unsaturated cyclic alkenes, including norbornenes and N-maleimides. Primers mentioned to be applied to bones in a first step are, for example, 4-hydroxy-3-methoxybenzaldehyde, 3,4-dihydroxy-phenethylamine, p-ethylphenol, p-vinylphenol, p-methacrylatephenol, bicyclo[2.2.1]hept-2-enoic acid, and various polyhydroxystyroles.

WO 2018/077973 A1 and Granskog et al., Adv. Funct. Mater. 28(26), 1800372 (2018), describe the synthesis of primer molecules and fiber-reinforced adhesive patches (FRAP) in a two-component adhesive system using a Tec-based resin formulation with tris[2-(3-mercaptopropionyloxy)ethyl]isocyanurate (TEMPIC) as a trifunctional thiol and triallylisocyanurate (TATATO) as a trifunctional monomer. In addition, the flexural modulus increases with the addition of hydroxyapatite as a filler. A formulation with a primer comprises 2,2-bis(allyloxymethyl)propanoic acid (BAPA), ethoxylated trimethylolpropane-tris(3-mercaptopropionate) (ETTMP), and a primer molecule containing a phosphonic acid that comprises one or two phosphonic acid groups each bound to BAPA, i.e., ((2,2-bis(allyloxymethyl)propanamido)methyl)phosphonic acid (herein referred to as BAPA^{Phn}) or ((((2,2-bis(allyloxymethyl)propanamido)ethyl)azanediyl)bis(methylene))-bis(phosphonic acid) (herein BAPA^{bisPhn}). For a matrix formulation with hydroxyapatite in combination with BAPA^{bisPhn} in the primer formulation, a shear bond strength of approximately 9 MPa is reported after curing.

Instead of phosphonic acid groups, various polyols, including moieties containing phenolic OH groups such as pyrocatechol or catechol groups in so-called “biomimetic primers” (due to their structural relation to the catecholamines of adrenaline and dopamine), as well as amines and silanes were used as adhesion motifs AM to increase adhesion in, for example, wet conditions, with silanes more often being used as primers for metal substrates. See, e.g., WO 2011/048077 A2; Granskog et al., v.s.; and Tomas Romson, “Triazine-based adhesive: An adherence study on clinically used metal surfaces”, Dissertation, Royal Institute of Technology, Stockholm, Sweden, 2018). When using pyrocatechol groups as AM-containing primer molecules for fiber-reinforced adhesive patches, the literature reports a shear bond strength of 0.29 MPa (Olofsson et al., RSC Adv. 6(31), 26398-26405 (2016)).

Ye at al. (“Surface-Initiated Ring-Opening Metathesis Polymerization of Pentadecafluorooctyl-5-norbornene-2-carboxylate from Variable Substrates Modified with Sticky Biomimic Initiator”, Macromolecules 43(13), 5554-5560 (2010)) disclose the use of the following primer, N-[2-(3,4-dihydroxyphenyl)ethyl]-5-norbornene-2-carboxylic acid amide (hereinafter referred to as “Catechol-2C-Norb”, Compound B2), with a pyrocatechol group as an adhesion motif AM and a norbornene group as a polymerizable group GP for impregnating metal and metal oxide surfaces:

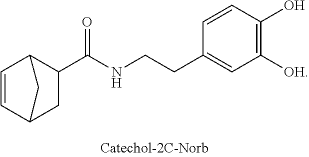 [SMILES: O=C(NCCc1ccc(O)c(O)c1)C1CC2C=CC1C2]

Specifically, TiO₂ nano wires were impregnated with the primer, immersed in a solution of a ruthenium catalyst and then in a solution of the norbornene monomer mentioned in the title, each in dichloromethane, so that the polymerization of the monomer was initiated and corresponding polymer chains were grafted onto the primer simultaneously serving as an initiator.

And WO 89/06666 A1 discloses thiol-ene-based compositions for manufacturing adhesives by thermal polymerization, which comprise, for example, in addition to numerous other compounds, those with norbornyl moieties as polymerizable groups PG and phosphoric acid esters as adhesion motifs AM as “adhesion-providing agents”. Substrates mentioned are metal and other polar substrates, with zinc, copper and steel surfaces having specifically been coated.

Despite all the different approaches and chemical concepts for the manufacture of bone adhesives and the partial overcoming of problems such as lower adhesion to wet surfaces and lower biocompatibility, key factors, such as simple applicability and surgical applicability, of such adhesives have insufficiently been taken into account. Against this background, it was the object of the invention to develop a thiol-ene-based polymerizable composition that is suitable for use as a bone adhesive and which at least partially solves some of the problems still existing.

### DISCLOSURE OF THE INVENTION

The present invention achieves this object by providing the use of compounds comprising, per molecule, at least one polymerizable C—C double bond, at least one adhesion-providing moiety, and one at least bivalent hydrocarbon moiety in between as a spacer as primers in a thiol-ene-based polymerizable composition for improving the adhesion of the composition on a substrate to be coated therewith, wherein the composition further comprises a radical initiator, monomers with at least two polymerizable C—C double bonds per molecule, and polythiols with at least two SH groups per molecule, wherein the primers comprise 5-norbornen-2-yl groups as polymerizable C—C double bonds, wherein the inventive use is characterized by the use, as primers in the polymerizable composition, of compounds
a) comprising phosphonic acid groups as adhesion-providing moieties; or
b) comprising groups selected from phosphonic acid groups and 3,4-dihydroxyphenyl groups as adhesion-providing moieties, and that the polymerizable composition is used as a bone adhesive.

The inventors have surprisingly found out the combinations of 5-norbornen-2-yl groups as polymerizable groups PG and phosphonic acid or 3,4-dihydroxyphenyl (4-pyrocatechyl) groups as adhesion motifs AM for the primer molecules show a synergistic effect and a number of advantages, not only, but particularly when using the composition as a bone adhesive. On the one hand, norbornenyl groups have shown to be the most reactive, directly compared to all other common primer PGs, in compositions curable by thiol-ene addition polymerization. On the other hand, the adhesion of the cured composition on substrates—on hydroxyapatite as well as on metal surfaces—achieved with these primers is clearly superior to that obtained with common primers in otherwise identical test compositions or with commercially available bone adhesives, as will be shown in the examples below by measurements of the shear bond strength of the cured compositions—for one—as well as two-component compositions.

Without wishing to be bound by theory, the inventors assume that the excellent results with regard to shear bond strength are also due to the high reactivity of the norbornenyl groups during thiol-ene addition polymerization. In this way, the primer molecules are integrated into the resulting polymer matrix immediately after initiation of the polymerization reaction and thus able to promote the adhesion of the matrix on the substrate to be coated, e.g., bone material, at a very early state, i.e., long before the curing of the composition is completed.

In preferred embodiments of the invention, the primers each comprise at least two 5-norbornen-2-yl groups and/or at least two adhesion-providing moieties, which further promotes the above effects, however, it is more preferable to have not more than three 5-norbornen-2-yl groups and/or not more than two adhesion-providing moieties because no further improvement of the effect is to be expected in these cases, so that the molecular weight of the primer and thus the mass of the formulation would be increased unnecessarily. In particular, the primers each comprise two 5-norbornen-2-yl groups and one or two adhesion-providing moieties, in particular phosphonic acid groups (HO)₂P(═O)—.

When using the compositions as bone adhesives, the adhesion-providing moieties AM of the primers are, in fact, in particular phosphonic acid groups because they showed even better results in shear bond strength tests after coating and curing of the composition on hydroxyapatite surfaces than 3,4-dihydroxyphenyl (4-pyrocatechyl) groups as AM of the primers and are easier to synthesize.

The spacer moiety for connecting the PG and AM groups of the primers are usually hydrocarbon moieties having a number of carbon atoms and optionally heteroatoms that is not particularly limited, so that oligomeric or polymeric spacers may also be used and may thus sometimes also connected a two-digit number of 5-borbornen-2-yl PG groups and/or AM moieties with each other. On the other hand, the spacers may also comprise only one or two (hydrocarbon) atoms to connect, for example, only one 5-norbornen-2-yl group and one AM group. According to the present invention it is, however, preferred that the spacer moiety has a chain length of at least 3 or at least 6 carbon atoms, one or several of which are optionally replaced by a heteroatom selected from O, S and N. Primers containing spacers with short chain lengths tend to result in lower shear bond strengths, which the inventors attribute, without wishing to be bound by theory, to a lower flexibility of the spacer-AM groups projecting from the polymer matrix after curing.

Spacer moieties of the primers having a number of carbon atoms, one or more of which are optionally replaced by a heteroatom selected from O, S and N, of more than 40 and/or a chain length, herein referring to the direct linear connection between the PG and AM groups, of more than 10 or more than 12 carbon atoms or heteroatoms are, however, also not to be preferred in the composition of the invention because they do not result in any additional increase of shear bond strength, but again undesirably increase the molecular weight and thus the mass of the formulation.

According to the invention, the monomers are preferably selected from compounds with at least two allyl or vinyl ester groups, more preferably from 1,3,3-triallyl-1,3,5-triazine-2,4,6-trione (TATATO), O,O′-(hexahydrofuro[3,2-b]furan-3,6-diyl)divinyl adipate (glucitol divinyl adipate, GDVA) and mixtures thereof because they have been shown to be well compatible with the inventive primers and resulted in good shear bond strengths in previous tests of the inventors. In particular, the monomers are or comprise 1,3,3-triallyl-1,3,5-triazine-2,4,6-trione (TATATO), which led to even better results in tests than O,O′-(hexahydrofuro[3,2-b]furan-3,6-diyl)divinyl adipate (GDVA).

In further preferred embodiments of the invention, the polythiols contained in the composition consist of compounds with at least three SH groups per molecule, particularly of tris[2-(3-mercaptopropionyloxy)ethyl]isocyanurate (TEMPIC), trimethylolpropane-tris(3-mercaptopropionate) (TMPMP) and mixtures thereof because these polythiols led to particularly good test results, which were even slightly better when tris[2-(3-mercaptopropionyloxy)ethyl]isocyanurate (TEMPIC) was used alone compared to trimethylolpropane-tris(3-mercaptopropionate) (TMPMP) alone or mixtures of both.

According to the invention, the polymerizable composition may be present as a one-component composition or a two-component composition, of which only the first component comprises one or more of the primers defined above, which is first applied to the surface to be coated and pre-cured, followed by the application of the second component on the first one and then complete curing of both layers. Depending on the particular application of the composition, either a one- or a two-component composition may be preferred.

The mass ratios of the components contained in the polymerizable composition are, of course, not particularly limited. A person with average skill in the field of thiol-ene polyaddition will, based on the teachings disclosed herein, be able to determine, without undue experimentation, optimum mixing rations between initiators, monomers, polythiols, primers and any additives, such as fillers, stabilizers and other additives commonly used in the field, for a particular selection of components and surfaces to be coated—for example, by conducting the test method described in the examples below.

In general, the polymerizable composition must, in preferred embodiments of the invention, comprise approximately 0.1 wt % to approximately 5 wt % of a radical initiator, approximately 10 wt % to approximately 50 wt % of monomers, approximately 40 wt % to approximately 80 wt % of polythiols, and approximately 5 wt % to approximately 25 wt % of primers, the sum of which must, of course, be 100 wt %. Additional additives, fillers, stabilizers, etc. are, of course, also possible in any amounts compatible with the other components in these cases.

In particularly preferred embodiments, the radical initiator of the invention is a radical photoinitiator, so that the composition is photopolymerizable. This is particularly advantageous with regard to the use as a bone adhesive because the application of the polymerizable composition for treating or healing in vivo is greatly simplified when curing of a composition applied to a bone fracture site can be initiated via simple irradiation. In such cases the polymerizable composition is, in particular, preferably a one-component composition. Alternatively, the initiators can also be redox or thermal initiators, wherein the latter are unlikely to be considered for in-vivo applications.

In particular, the invention thus provides the use of a composition as defined above, in particular of a one-component composition, for use as a bone adhesive usable by medical professionals for treating bone fractures or injuries.

In a second aspect, the present invention provides a novel norbornene derivative of the formula

(Norb)ₙ-Sp-(AM)ₘ

wherein
Norb represents a 5-norbornen-2-yl radical;
each AM independently represents a phosphonic acid group (HO)₂P(═O)— or a 3,4-dihydroxyphenyl group;
n and m each independently represent an integer ≥1; and
Sp represents a spacer group selected from (n+m)-valent hydrocarbon residues with 1 to 40 carbon atoms, one or more of which are optionally replaced by a heteroatom selected from O, S and N;
provided that at least one of n and m represents an integer >1 when AM represents a 3,4-dihydroxyphenyl group.

These novel chemical compounds with one or more 5-norbornen-2-yl radicals as polymerizable groups and one or more phosphonic acid groups (HO)₂P(═O)— and/or 3,4-dihydroxyphenyl groups as adhesion-providing moieties AM are very well suited as primers in thiol-ene based polymerizable compositions according to the first aspect of the present invention, as will be shown by numerous examples and comparative examples below.

The number of polymerizable 5-norbornen-2-yl radicals Norb and phosphonic acid moieties AM in the norbornene derivatives according to the present invention is not particularly limited. This means that depending on the chain length of the spacer Sp, only one or several groups Norb and (HO)₂P(═O)— can be bound to these, e.g., 2, 3, 4, 5, or more Norb and/or (HO)₂P(═O)— groups each, wherein values of 2 or more are preferred for n and/or m according to the invention. In the case of 3,4-dihydroxyphenyl moieties as adhesion motifs AM, the inventive norbornene derivatives are, however, limited to those in which the number n of Norb groups and/or the number m of 3,4-dihydroxyphenyl moieties AM is at least 2 because these led to clearly better results in the examples than those in which n and m were each only 1 (such as in the primer Catechol-2C-Norb, Compound B2, from Example 21 known from literature).

As mentioned before, even two-digit numbers of corresponding Norb and/or AM groups may be contained in the norbornene derivatives in the case of oligomeric or polymeric spacers. This means that n and m can each represent 2, 3, 4, 5, or more, sometimes even a number >10.

As also mentioned before, n and m preferably represent not more than 3 because with a larger number of Norb and AM no further improvements of the effect when norbornene derivatives are used as primers in polymerizable compositions are to be expected, so that the molecular weight of the primers and thus the mass of the respective formulation are unnecessarily increased.

Particularly preferably, the inventive norbornene derivative of the above formula is characterized in that
a) one or both of n and m represent(s) 2; and/or
b) S represents an (n+m)-valent hydrocarbon residue with 3 to 20 or 6 to 16 carbon atoms, one or more of which are optionally replaced by a heteroatom selected form O, S and N; and/or
c) AM represents a phosphonic acid group (HO)₂P(═O)—;

wherein, more preferably, all three characteristics a) to c) are met, which led to the best results.

And in preferred embodiments, the norbornene derivative of the present invention is selected form the following compounds, which have proven successful in the examples.

### N-(4-Hydroxy-4,4-diphosphonobutyl)-5-norbornene-2-carbonic acid amide sodium salt (N-(4-hydroxy-4,4-diphosphonobutyl)alendronic acid amide sodium salt) (Alendronate-Norb) (E1)

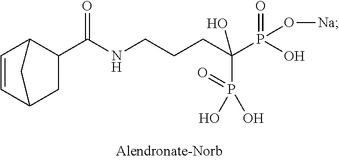

### 5-Norbornen-2-ylcarbonyloxymethylphosphonic acid (Phn-1C-Norb) (E3)

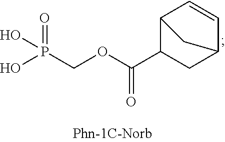 [SMILES: O=C(OCP(=O)(O)O)C1CC2C=CC1C2]

### 6-(5-Norbornen-2-ylcarbonyloxy)hexylphosphonic acid (Phn-6C-Norb) (E4)

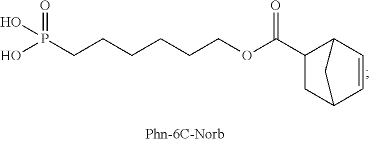 [SMILES: O=C(OCCCCCCP(=O)(O)O)C1CC2C=CC1C2]

### 6-(2,3-Bis(5-norbornen-2-ylcarbonyloxy)propylthio)hexylphosphonic acid (Phn-6C-bisNorb) (E5)

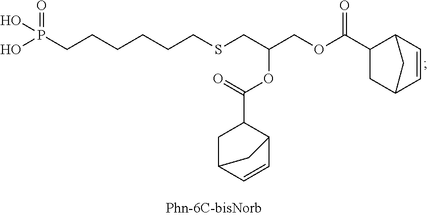 [SMILES: O=C(OCC(CSCCCCCCP(=O)(O)O)OC(=O)C1CC2C=CC1C2)C1CC2C=CC1C2]

### 6-(2,3-Bis(5-norbornen-2-ylcarbonyloxy)propylthio)hexane-1,1-diyl-bis(phosphonic acid) (bisPhn-6C-bisNorb) (E6)

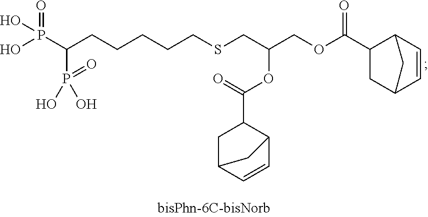 [SMILES: O=C(OCC(CSCCCCCC(P(=O)(O)O)P(=O)(O)O)OC(=O)C1CC2C=CC1C2)C1CC2C=CC1C2]

### 3-(3-(3,4-Dihydroxyphenyl)propylthio)propane-1,2-diyl-bis(norbornene-2-carboxylate) (Catechol-3C-bisNorb) (E7)

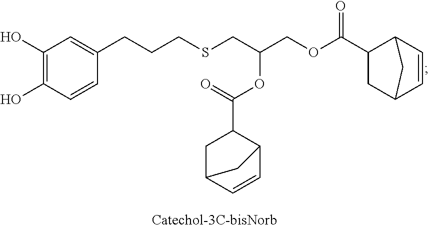 [SMILES: O=C(OCC(CSCCCc1ccc(O)c(O)c1)OC(=O)C1CC2C=CC1C2)C1CC2C=CC1C2]

### Ethylenediaminetetraacetic acid-bis(2-(3,4-dihydroxyphenyl)ethyl)amide-bis(2-(5-norbornen-2-yl)ethyl)amide (bisCatechol-EDTA-bisNorb) (E8)

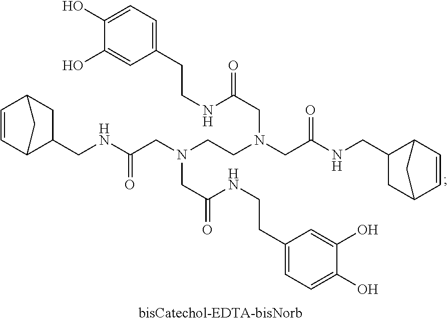 [SMILES: O=C(CN(CCN(CC(=O)NCCc1ccc(O)c(O)c1)CC(=O)NCC1CC2C=CC1C2)CC(=O)NCC1CC2C=CC1C2)NCCc1ccc(O)c(O)c1]

### Diethylenetriaminepentaacetic acid-bis(2-(5-norbornen-2-yl)ethyl)amide-tris(2-(3,4-dihydroxyphenyl)ethyl)amide (triCatechol-DTPA-bisNorb) (E9)

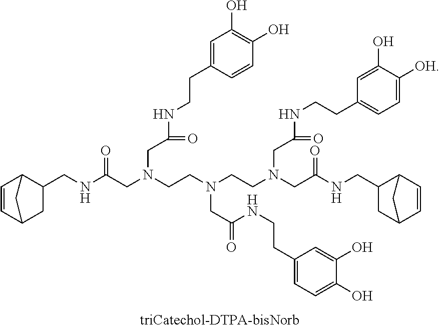 [SMILES: O=C(CN(CCN(CC(=O)NCCc1ccc(O)c(O)c1)CC(=O)NCC1CC2C=CC1C2)CCN(CC(=O)NCCc1ccc(O)c(O)c1)CC(=O)NCC1CC2C=CC1C2)NCCc1ccc(O)c(O)c1]

Of these novel compounds, 6-(2,3-bis(5-norbornen-2-ylcarbonyloxy)propylthio)hexylphosphonic acid (Phn-6C-bisNorb) (E5) and 6-(2,3-bis(5-norbornen-2-ylcarbonyloxy)propylthio)hexane-1,1-diyl-bis(phosphonic acid) (bisPhn-6C-bisNorb) (E6) have proven to be especially effective.

Due to these good results, the present invention also provides the use of the above novel norbornene derivatives as primers in polymerizable compositions according to the first aspect.

### SHORT DESCRIPTION OF THE DRAWINGS

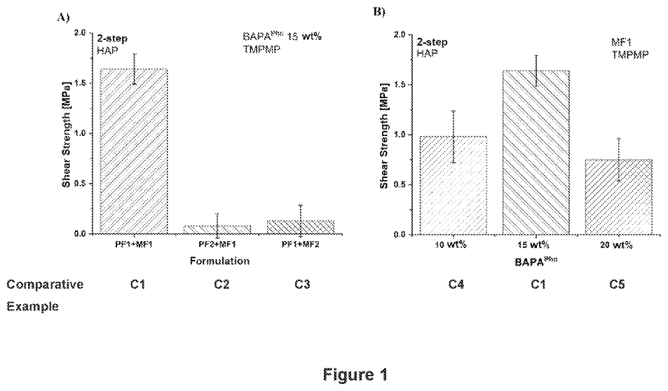

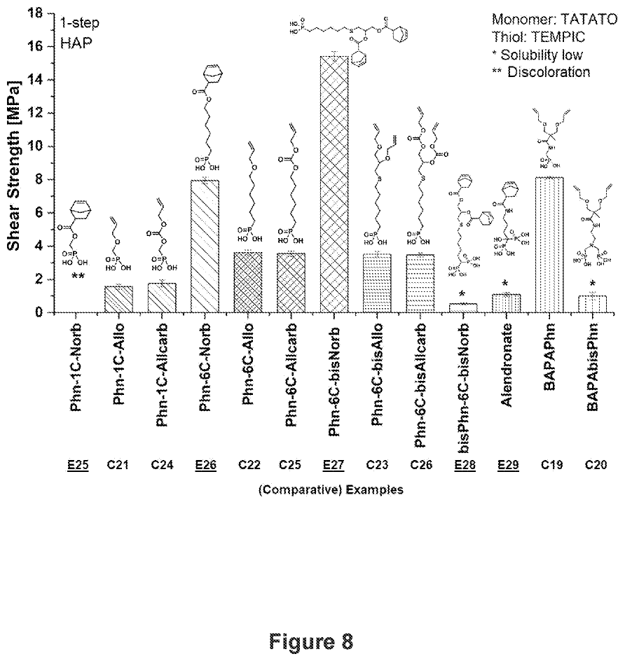

Below, the present invention will be described in further detail based on specific synthesis, embodiment and comparative examples, which are exclusively for illustrative purposes and not to be understood as limiting, as well as with reference to the attached figures. Of these, FIGS. 1 to 7 are graphic representations of the results of shear bond strength measurements that were achieved with two-component compositions of Examples 1 to 24 and Comparative Examples 1 to 18, and FIGS. 8 to 12 are representations of the results achieved with one-component compositions of Examples 25 to 36 and Comparative Examples 19 to 30.

### EXAMPLES

Unless otherwise specified herein, all reagents reacted in the reactions below were obtained from commercial sources in synthesis grade and used without any further purification. The purity of all intermediates and products obtained in the examples was examined by determining physical data such as melting point (Fp.) and refractive index (RI), as well as by nuclear resonance spectra (1H, 13C and ³¹P NMR), high-resolution mass spectrometry (HR-MS), and elementary analyses. All norbornenyl-containing compounds were racemic mixtures of the respective endo- and exo-isomers.

The following synthesis examples are grouped depending on the polymerizable groups PG and adhesion motifs AM that were introduced.

### SYNTHESIS EXAMPLES

### Synthesis Examples 1 to 8—Preparation of Comparative Primers with Allyloxy (Allo) or Allyloxycarbonyloxy (Allcarb) as Pg and Phosphonic Acid (Phn) as am

### Synthesis Example 1—Preparation of ((2,2-bis(allyloxymethyl)propaneamido)methyl)phosphonic acid (BAPA^{Phn}) (S1)

### Step 1: Preparation of 2,2-bis(allyloxymethyl)propanoic acid (BAPA)

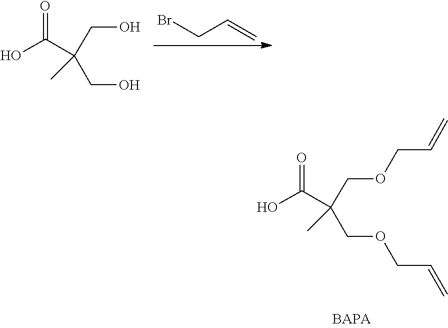

The synthesis known from literature was carried out as described in Antoni et al., Macromolecules 43, 6625-6631 (2010) by reacting 2,2-bis(hydroxymethyl)propanoic acid with allylbromide.

DC (PE:EE, 9:1) R_{f}=0.3; RI n_{D}^{20° C.}: 1.4566; ¹H NMR (400 MHZ, CDCl₃) δ (ppm): 6.00-5.77 (m, 2H, 2×CH═CH₂), 5.22 (dd, 4H, 2×CH═CH₂), 4.01 (d, ³J_{HH}=5.5, 1.5 Hz, 4H, 2×OCH₂CH), 3.58 (s, 4H, 2×CCH₂O): 1.25 (s, 3H, CCH₃); ¹³C-NMR (101 MHZ, CDCl₃) δ (ppm): 180.14 (s, COOH), 136.7 (s, CH═CH₂), 117.08 (s, CH═CH₂), 72.55 (s, CCH₂O), 72.01 (s, OCH₂CH), 48.24 (s, C_{q}), 17.97 (s, CCH₃).

### Step 2—Preparation of ((2,2-bis(allyloxymethyl)propaneamido)methyl)phosphonic acid (BAPA^{Phn}) (S1)

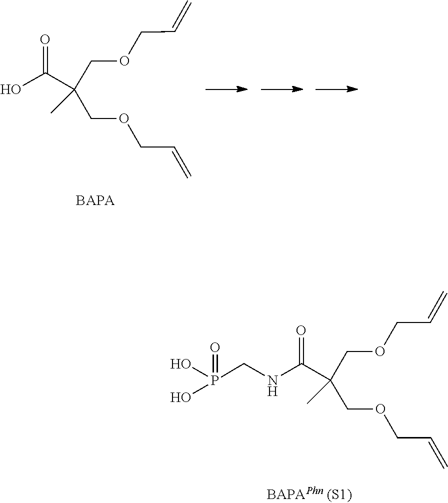

The synthesis known from literature was carried out as described in Granskog et al., v.s., by activating BAPA with carbonyldiimidazole (CDI), followed by reacting the mixed anhydride obtained as an intermediate product with aminoethyl phosphonic acid diethyl ester and final ester cleavage with bromotrimethylsilane.

¹H NMR (400 MHZ, CDCl₃) δ (ppm): 6.02-5.73 (m, 2H, 2×CH═CH₂), 5.35-5.08 (m, 4H, 2×CH═CH₂), 4.01 (m, 4H, 2×CH₂CH═CH₂), 3.72-3.64 (m, 2H, NHCH₂P), 3.55 (s, 4H, 2×CCH₂O), 1.19 (s, 3H, CCH₃); ¹³C-NMR (101 MHZ, CDCl₃) δ (ppm): 134.34 (s, CH═CH₂), 117.70 (s, CH═CH₂), 73.28 (s, CCH₂O), 72.62 (s, 2×CH₂CH═CH₂), 47.79 (s, C_{q}), 34.56 (d, ¹J_{CP}=155.3 Hz, NHCH₂P), 18.38 (s, C_{q}CH₃); ³¹P NMR (101 MHZ, CDCl₃) δ (ppm): 19.22.

### Synthesis Example 2—Preparation of ((((2,2-bis(allyloxymethyl)propanamido)ethyl)azanediyl)-bis(methylene))-bis(phosphonic acid) (BAPA^{bisPhn}) (S2)

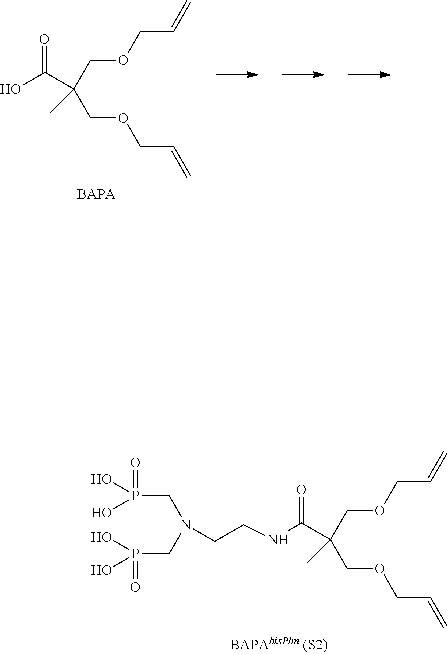 [SMILES: C=CCOCC(C)(COCC=C)C(=O)O ; C=CCOCC(C)(COCC=C)C(=O)NCCN(CP(=O)(O)O)CP(=O)(O)O]

The synthesis known from literature was also carried out according to Granskog et al., v.s., again by first activating the BAPA obtained in the Synthesis Example, Step 1, BAPA with carbonyldiimidazole (CDI), reacting the mixed anhydride with ethylene diamine to obtain aminoethylamide, which was reacted with 2 mol p-formaldehyde and dimethyl phosphite to obtain bis(phosphonic acid dimethyl ester), and again followed by a final ester cleavage with bromotrimethylsilane.

¹H NMR (400 MHZ, CDCl₃) δ (ppm): 8.10 (s, 1H, NHCO), 6.12-5.81 (m, 2H, CH═CH₂), 5.21 (m, 4H, 2×CH═CH₂), 3.97 (d, 4H, ³J_{HH}=12.9 Hz, 2×CH₂CH═CH₂), 3.80 (d, ³J_{HH}=8.2 Hz, 4H, 2×NCH₂P), 3.70 (m, 4H, NHCH₂CH₂N), 3.54 (m, 4H, 2×CCH₂O), 1.21 (s, 3H, CCH₃); ¹³C-NMR (101 MHZ, CDCl₃) δ (ppm): 178.9 (s, C═O), 136.2 (s, CH═CH₂), 117.40 (s, CH═CH₂), 73.80 (s, CCH₂O), 72.89 (s, 2×CH₂CH═CH₂), 59.4 (s, NHCH₂CH₂N), 52.9+51.5 (s, 2×NCH₂P), 48.98 (s, C_{q}), 36.71 (s, NHCH₂CH₂N), 18.29 (s, C_{q}CH₃); ³¹P NMR (101 MHZ, CDCl₃) δ (ppm): 9.66.

### Synthesis Example 3—Preparation of Allyloxymethylphosphonic Acid (Phn-1C-Allo) (S3)

### Step 1: Preparation of Allyloxymethylphosphonic Acid Diethylester (PhnEt-1C-Allo)

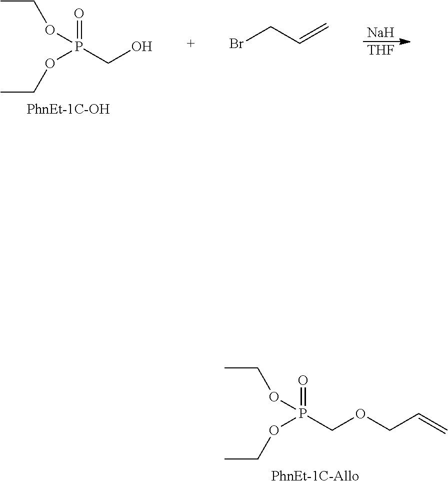

In a three-necked flask, sodium hydride (1.9 Äqu., 0.7 g, 28.5 mmol) was suspended in 40 ml of dry tetrahydrofuran (THF) under argon. Hydroxymethylphosphonic acid diethylester (PhnEt-1C—OH) (1 eq., 2.2 ml, 15 mmol) was diluted in 10 ml of THF and slowly added at 0° C. via a septum. After 1 h, allylbromide (1.3 eq., 1.7 ml, 19.5 mmol) was injected into the flask. Then, the ice bath was removed, and the reaction mixture was stirred over night at room temperature. After evaporation of the solvent and non-reacted educts, the raw material was purified by column chromatography (ethyl acetate, EE), 1.85 g (59% of theory) of the desired phosphonates PhnEt-1C-Allo were isolated as a colorless liquid.

DC (EE) R_{f}=0.52; RI n_{D}^{20° C.}: 1.4357; ¹H NMR (400 MHZ, CDCl₃) δ (ppm): 5.96-5.77 (m, 1H, CH₂CH═CH₂), 5.33-5.18 (m, 2H, CH₂CH═CH₂), 4.25-4.03 (m, 6H, 2×OCH₂CH₃, CH₂CH═CH₂), 3.75 (d, ³J_{HH}=8.7 Hz, 2H, PCH₂O), 1.39-1.29 (t, ³J_{HH}=7.0 Hz, 6H, 2×OCH₂CH₃); ¹³C-NMR (101 MHZ, CDCl₃) δ (ppm): 133.78 (s, OCH₂CHCH₂), 118.54 (s, OCH₂CHCH₂), 74.11 (d, J=12.7 Hz, OCH₂CHCH₂), 63.81 (d, ²J_{CP}=167.1 Hz, CH₂P C₁), 62.54 (d, ³J_{CP}=6.3 Hz, OCH₂CH₃), 16.53 (d, ³J_{CP}=6.0 Hz, OCH₂CH₃): ³¹P NMR (CDCl₃) δ (ppm): 21.58.

### Step 2: Preparation of Allyloxymethylphosphonic Acid (Phn-1C-Allo) (S3)

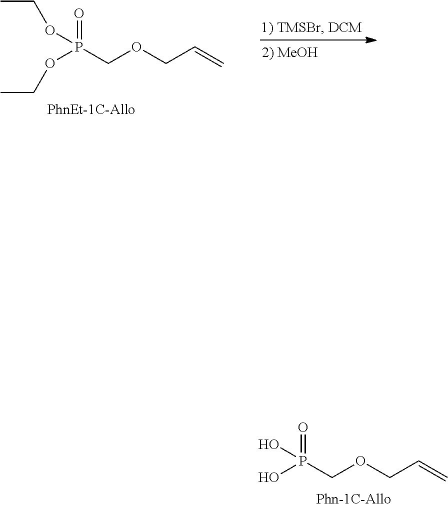

1.7 ml of bromotrimethylsilane (TMSBr) (12.5 mmol, 2.5 eq.) were added to a solution of PhnEt-1C-Allo (1 eq., 1.1 g, 5 mmol) in 15 ml of dry methylene chloride (DCM). After 20 h stirring at 30° C., the mixture was concentrated under reduced pressure. Methanol (20 ml) was added, and the solution was stirred at room temperature for 5 h. The solvent was evaporated, and the product was dried under vacuum. 0.75 g (99% of th.) of the desired phosphonic acid Phn-1C-Allo were isolated as a colorless liquid.

RI n_{D}^{20° C.}: 1.4773; ¹H NMR (400 MHZ, CDCl₃) δ (ppm): 9.78 (s, 2H, 2×OH), 5.97-5.78 (m, 1H, CH₂CH═CH₂), 5.41-5.21 (m, 2H, CH₂CH═CH₂), 4.15 (d, J=5.7 Hz, 2H, CH₂CH═CH₂), 3.85 (d, J=8.8 Hz, 2H, PCH₂O); ¹³C-NMR (101 MHZ, CDCl₃) δ (ppm): 133.19 (s, OCH₂CHCH₂), 119.51 (s, OCH₂CHCH₂), 74.44 (d, J=12.5 Hz, OCH₂CHCH₂), 63.81 (d, ²J_{CP}=167.9 Hz, CH₂P C₁); ³¹P NMR (CDCl₃) δ (ppm): 23.73; HR-MS (MeOH, ESI, m/z): calcd. 152.09, found 152.07 [M].

### Synthesis Example 4—Preparation of 6-allyloxyhexylphosphonic acid (Phn-6C-Allo) (S4)

Step 1: Preparation of 6-allyloxyhexylphosphonic acid diethylester (PhnEt-6C-Allo)

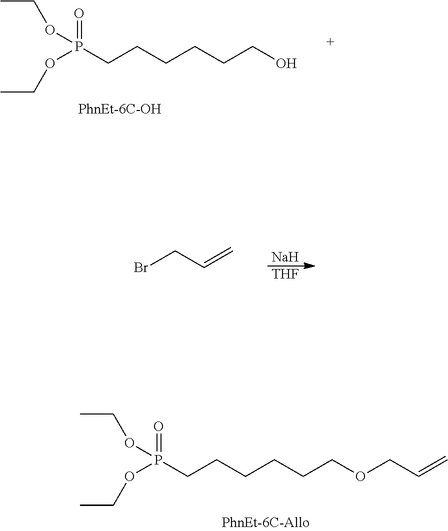

The synthesis was conducted in analogy to Synthesis Example 3, Step 1, from 6-hydroxyhexylphosphonic acid (PhnEt-6C—OH) (1 eq., 3.1 g, 13 mmol) with allyl bromide (1.5 eq., 1.7 ml, 19.5 mmol) and sodium hydride in paraffin oil (1.9 eq., 0.6 g, 24.7 mmol). By means of column chromatography (EE), 1.4 g (49% of th.) of the desired phosphonate PhnEt-6C-Allo were isolated as a colorless liquid.

DC (EE/MeOH, 19:1) R_{f}=0.47; RI n_{D}^{2° C.}: 1.4461; ¹H NMR (400 MHZ, CDCl₃) δ (ppm): 5.98-5.81 (ddt, ¹J_{HH}=17.3, ²J_{HH}=10.4, ³J_{HH}=5.6 Hz, 1H, CH═CH₂), 5.24 (dq, ¹J_{HH}=17.2, ²J_{HH}=1.7 Hz, 1H, CH═CH₂). 5.15 (dq, ¹J_{HH}=10.4, ²J_{HH}=1.4 Hz, 1H, CH═CH₂), 4.16-4.00 (m, 4H, 2×OCH₂CH₃), 3.94 (dt, ¹J_{HH}=5.8, ²J_{HH}=1.4 Hz, 2H, OCH₂CH), 3.40 (1, ²J_{HH}=6.6 Hz, 2H, CH₂OCH₂CH), 1.77-1.48 (m, 6H, CH₂P C₁, CH₂CH₂O C₅. CH₂C₄), 1.42-1.20 (m, 10H, 2×OCH₂CH₃, CH₂C₃, CH₂CH₂P); ¹³C-NMR (101 MHZ, CDCl₃) δ (ppm): 135.17 (s, CH₂CH═CH₂), 116.83 (s, CH₂CH═CH₂), 71.92 (s, CH₂CH═CH₂), 70.37 (s, CH₂O), 61.49 (d, ²J_{CP}=6.6 Hz, OCH₂CH₃), 30.55 (d, ³J_{CP}=16.8 Hz, CH₂CH₂CH₂P), 29.63 (s, CH₂CH₂O C₅), 25.84 (s, ⁴J_{CP}=1.3 Hz, CH₂C₄), 25.76 (d, ¹J_{CP}=140.6 Hz, CH₂P C₁), 22.50 (d, ²J_{CP}=5.1 Hz, PCH₂CH₂), 16.62 (d, ³J_{CP}=6.0 Hz, OCH₂CH₃); ³¹P NMR (CDCl₃) δ (ppm): 32.55.

### Step 2: Preparation of 6-allyloxyhexylphosphonic acid (Phn-6C-Allo) (S4)

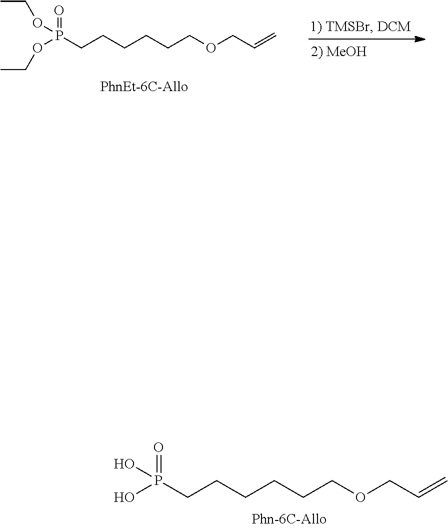 [SMILES: C=CCOCCCCCCP(=O)(OCC)OCC ; C=CCOCCCCCCP(=O)(O)O]

The synthesis was conducted in analogy to Synthesis Example 3, Step 2, from PhnEt-6C-Allo (1 eq., 1.4 g, 5 mmol) with TMSBr (2.5 eq., 1.7 ml, 12.5 mmol) to isolate 0.95 g (99% d. Th.) of the desired phosphonic acid Phn-6C-Allo as a brown viscous liquid. RI n_{D}^{2° C.}: 1.4775; ¹H NMR (400 MHZ, CDCl₃) δ (ppm): 9.03-8.47 (m, 2H, 2×OH), 5.91 (ddt, ¹J_{HH}=17.2, ²J_{HH}=10.3, ³J_{HH}=5.7 Hz, 1H, CH═CH₂), 5.27 (dq, ¹J_{HH}=17.2, ²J_{HH}=1.6 Hz, 1H, CH═CH₂), 5.18 (dq, ¹J_{HH}=10.4, ²J_{HH}=1.4 Hz, 1H, CH═CH₂), 3.98 (dt, ¹J_{HH}=5.7, ²J_{HH}=1.4 Hz, 2H, OCH₂CH), 3.45 (t, ³J_{HH}=6.6 Hz, 2H, CH₂OCH₂CH), 1.88-1.73 (m, 2H, PCH₂), 1.62 (m, 4H, CH₂CH₂O C₅, CH₂C₄), 1.40 (m, 4H, PCH₂CH₂C₂, PCH₂CH₂CH₂C₃); ¹³C-NMR (101 MHZ, CDCl₃) δ (ppm): 134.70 (s, CH₂CH═CH₂), 117.38 (s, CH₂CH═CH₂), 71.97 (s, CH₂CH═CH₂), 70.43 (s, CH₂O), 30.15 (d, ³J_{CP}=16.9 Hz, CH₂CH₂CH₂P), 29.35 (s, CH₂CH₂O C₅), 25.65 (s, CH₂C₄), 25.53 (d, ¹J_{CP}=141 Hz, CH₂P C₁), 21.96 (d, ²J_{CP}=5.1 Hz, PCH₂CH₂C₂); ³¹P NMR (CDCl₃) δ (ppm): 36.26; HR-MS (MeOH, ESI, m/z): calcd. 222.22, found 222.20 [M].

### Synthesis Example 5—Preparation of 6-(2,3-bis(allyloxy)propylthio)hexylphosphonic acid (Phn-6C-bisAllo) (S5)

### Step 1: Preparation of 6-(2,3-bis(allyloxy)propylthio)hexylphosphonic acid diethylester (PhnEt-6C-bisAllo)

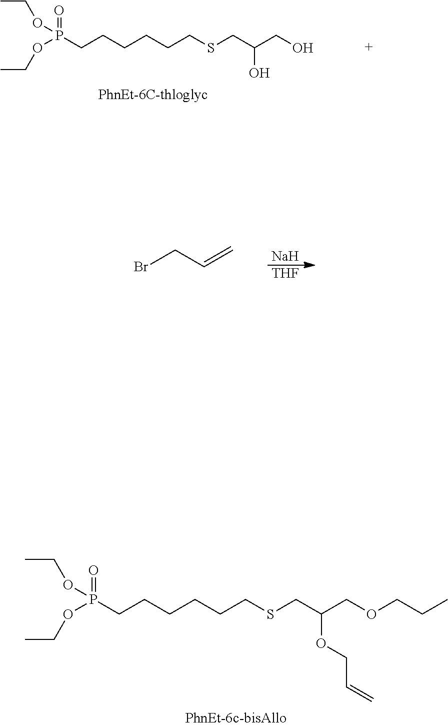

The synthesis was conducted in analogy to Synthesis Example 3, Step 1, from 6-(2,3-dihydroxypropylthio)hexylphosphonic acid diethylester (PhnEt-6C-thioglyc) (1 eq., 3.1 g, 13 mmol) with allyl bromide (1.5 eq., 1.7 ml, 19.5 mmol) and sodium hydride in paraffin oil (1.9 eq., 0.6 g, 24.7 mmol). By means of column chromatography (EE), 1.4 g (49% of th.) of the desired phosphonate PhnEt-6C-bisAllo were isolated as a colorless liquid.

DC (EE) R_{f}=0.3; RI n_{D}^{2° C.}: 1.4775; ¹H NMR (400 MHZ, CDCl₃) δ (ppm): 5.99-5.82 (m, 2H, CH═CH₂). 5.32-5.09 (m, 4H, CH═CH₂), 4.23-3.95 (m, 8H, OCH₂CH₃, OCH₂), 3.74-3.49 (m, 3H, CHO, CH₂O), 2.80-2.61 (m, 2H, alkyl-CH₂SCH₂), 2.59-2.47 (m, 2H, alkyl-CH₂SCH₂), 1.79-1.50 (m, 6H, PCH₂, PCH₂CH₂, CH₂CH₂CH₂S), 1.38 (m, 4H, PCH₂CH₂CH₂, CH₂CH₂S), 1.31 (t, ³J_{HH}=7.0 Hz, 6H, OCH₂CH₃); ¹³C-NMR (101 MHz, CDCl₃) δ (ppm): 134.93 (s, 2×CH═CH₂), 117.00 (s, 2×CH═CH₂), 78.2 (s, CHO), 72.35 (s, CH₂O), 71.18 (d, ³J_{CP}==3.6 Hz, 2×OCH₂CH═CH₂), 61.42 (d, ³J_{CP}=6.5 Hz, OCH₂CH₃), 33.61 (s, CH₂SCH₂), 33.05 (s, alkyl-CH₂SCH₂), 30.22 (d, ³J_{CP}=16.8 Hz, PCH₂CH₂CH₂), 29.44 (s, CH₂CH₂CH₂S), 28.33 (s, CH₂CH₂S), 25.67 (d, ¹J_{CP}=140.6 Hz, PCH₂), 22.36 (d, ²J_{CP}=5.2 Hz, PCH₂CH₂), 16.45 (d, ³J_{CP}=6.0 Hz, OCH₂CH₃); ³¹P NMR (CDCl₃) δ (ppm): 32.40.

### Step 2: Preparation of 6-(2,3-bis(allyloxy)propylthio)hexylphosphonic acid (Phn-6C-bisAllo) (S5)

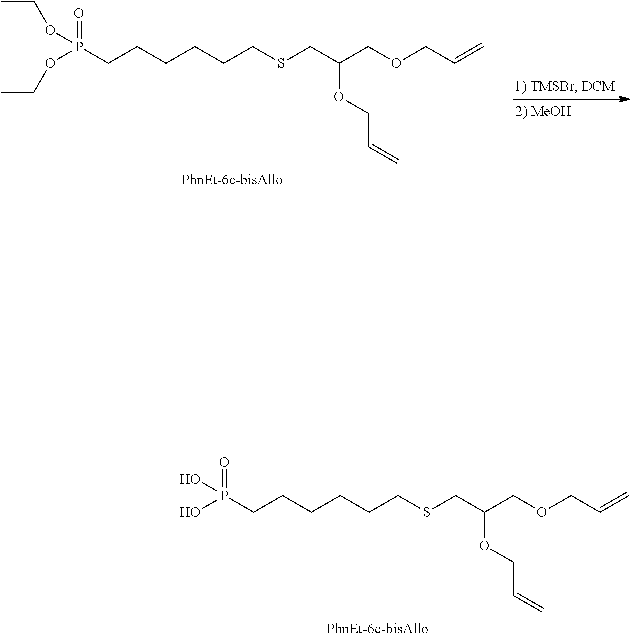 [SMILES: C=CCOCC(CSCCCCCCP(=O)(OCC)OCC)OCC=C ; C=CCOCC(CSCCCCCCP(=O)(O)O)OCC=C]

The synthesis was conducted in analogy to Synthesis Example 3, Step 1, from PhnEt-6C-bisAllo (1 eq., 0.8 g, 1.9 mmol) with TMSBr (2.5 eq., 0.6 ml, 4.75 mmol) to isolate 0.5 g (75% of th.) of the desired phosphonic acid Phn-6C-bisAllo as a brown viscous liquid.

RI n_{D}^{20° C.}: 1.5084; ¹H NMR (400 MHZ, CDCl₃) δ (ppm): 6.02-5.80 (m, 2H, CH═CH₂), 5.32-5.14 (m, 4H, CH═CH₂), 4.19-3.98 (m, 4H, OCH₂), 3.75-3.52 (m, 3H, CHO, CH₂ O), 2.77-2.62 (m, 2H, alkyl-CH₂SCH₂), 2.60-2.50 (m, 2H, alkyl-CH₂SCH₂), 1.87-1.70 (m, 2H, PCH₂), 1.70-1.49 (m, 4H, PCH₂CH₂; CH₂CH₂CH₂S), 1.45-1.33 (m, 4H, PCH₂CH₂CH₂, CH₂CH₂S); ¹³C-NMR (101 MHZ, CDCl₃) δ (ppm): 135.12 (s, CH═CH₂), 134.74 (s, CH═CH₂), 117.20 (s, 2×CH═CH₂), 78.10 (s, CHO), 72.51 (s, CH₂O), 71.30 (d, ³J_{CP}=3.7 Hz, 2×OCH₂CH═CH₂), 33.67 (s, CH₂SCH₂), 33.13 (s, alkyl-CH₂SCH₂), 30.22 (d, ³J_{CP}=17.1 Hz, PCH₂CH₂CH₂), 29.45 (s, CH₂CH₂CH₂S), 28.27 (s, CH₂CH₂S), 25.32 (d, ¹J_{CP}=141 Hz, PCH₂), 21.95 (d, ²J_{CP}=5.5 Hz, PCH₂CH₂); ³¹P NMR (CDCl₃) δ (ppm): 37.32; HR-MS (MeOH, ESI, m/z): calcd. 352.43, found 352.47 [M].

### Synthesis Example 6—Preparation of Allyloxycarbonyloxymethylphosphonic Acid (Phn-1C-Allcarb) (S6)

### Step 1: Preparation of Allyloxycarbonyloxymethylphosphonic Acid Diethylester (PhnEt-1C-Allcarb)

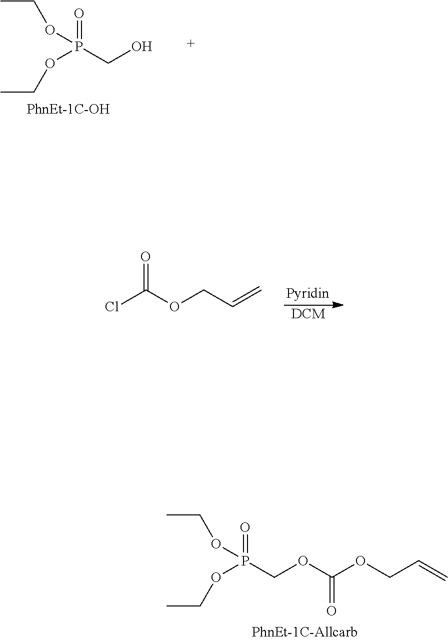

A three-necked flask was charged with hydroxymethylphosphonic acid diethylester (PhnEt-1C—OH) (1 eq., 2.2 ml, 15 mmol) and pyridine (4 eq., 4.8 ml, 60 mmol) in 50 ml of DCM under argon. Chloroformic acid allyl ester (1.3 eq., 2.1 ml, 19.5 mmol) was added dropwise at 0° C. via a septum. After 1 h of stirring at 0° C., the ice bath was removed, and the reaction was stirred at room temperature overnight. The raw product was dissolved in EE, and the precipitate (pyridinium salts) was collected by filtration. After evaporating the solvent, 2.9 g (77% of th.) of the desired phosphonate PhnEt-1C-Allcarb were isolated as a colorless liquid.

DC (EE) R_{f}=0.5; RI n_{f}^{20° C.}: 1.4460; ¹H NMR (400 MHZ, CDCl₃) δ (ppm): 6.00-5.85 (m, 1H, CH₂CH═CH₂). 5.43-5.22 (m, 2H, CH₂CH═CH₂), 4.69-4.61 (m, 2H, CH₂CH═CH₂), 4.43 (d, ³J_{HH}=8.5 Hz, 2H, PCH₂O), 4.25-4.10 (m, 4H, OCH₂CH₃), 1.35 (t, ³J_{HH}=7.0 Hz, 6H, OCH₂CH₃); ¹³C-NMR (101 MHZ, CDCl₃) δ (ppm): 154.71 (d, ³J_{CP}=9.5 Hz, OC═OO), 131.26 (s, OCH₂CHCH₂), 119.42 (s, OCH₂CHCH₂), 69.31 (s, OCH₂CHCH₂), 63.07 (d, ²J_{CP}=6.5 Hz, OCH₂CH₃), 60.32 (d, ¹J_{CP}=168.5 Hz, CH₂P C₁), 16.49 (d, ³J_{CP}=6.0 Hz, OCH₂CH₃); ³¹P NMR (CDCB) δ (ppm): 17.74.

### Step 2: Preparation of Allyloxycarbonyloxymethylphosphonic Acid (Phn-1C-Allcarb) (S6)

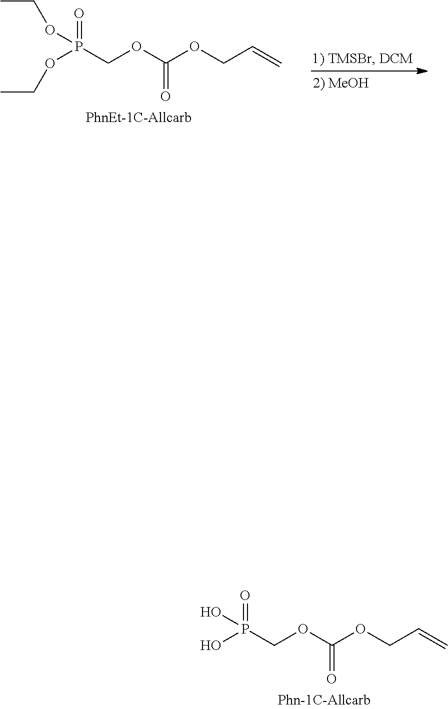 [SMILES: C=CCOC(=O)OCP(=O)(OCC)OCC ; C=CCOC(=O)OCP(=O)(O)O]

The synthesis was conducted in analogy to Synthesis Example 3, Step 2, from PhnEt-1C-Allcarb (1 eq., 2.5 g, 10 mmol) with TMSBr (2.5 eq., 3.3 ml, 25 mmol) to isolate 1.76 g (99% of th.) of the desired phosphonic acid Phn-1C-Allcarb as a colorless liquid. RI n_{D}^{2° C.}: 1.4566; ¹H NMR (400 MHZ, CDCl₃) δ (ppm): 9.52 (s, 2H, OH), 6.00-5.81 (m, 1H, CH₂CH═CH₂), 5.43-5.22 (m, 2H, CH₂CH═CH₂), 4.65 (d, ³J_{HH}=5.7 Hz, 2H, CH₂CH═CH₂), 4.46 (d, ³J_{HH}=8.8 Hz, 2H, PCH₂O); ¹³C-NMR (101 MHZ, CDCl₃) δ (ppm): 154.87 (d, ³J_{CP}=9.5 Hz, OC═OO), 131.19 (s, OCH₂CHCH₂), 119.58 (s, OCH₂CHCH₂), 69.64 (s, OCH₂CHCH₂), 61.19 (d, J_{CP}=169.5 Hz, CH₂P C₁); ³¹P NMR (CDCl₃) δ (ppm): 19.11; HR-MS (MeOH, ESI, m/z): calcd. 196.09, found 196.06 [M].

### Synthesis Example 7—Preparation of 6-(allyloxycarbonyloxy)hexylphosphonic acid (Phn-6C-Allcarb) (S7)

### Step 1: Preparation of 6-(allyloxycarbonyloxy)hexylphosphonic acid diethylester (PhnEt-6C-Allcarb)

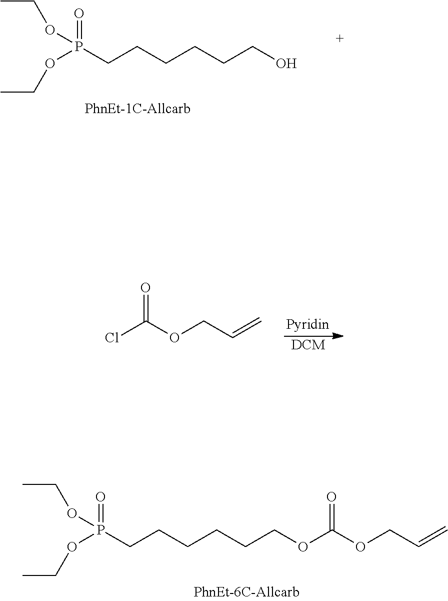

The synthesis was conducted in analogy to Synthesis Example 6, Step 1, from 6-hydroxyhexylphosphonic acid diethylester (PhnEt-6C—OH) (1 eq., 2.4 g, 10 mmol) with chloroformic acid allyl ester (1.5 eq., 1.6 ml, 15 mmol) and pyridine (4 eq., 3.2 ml, 40 mmol), wherein after evaporation of the solvent, 2.2 g (68% of th.) of the desired phosphonate PhnEt-6C-Allcarb were isolated as a yellowish viscous liquid.

DC (EE/MeOH, 9:1) R_{f}=0.51; RI n_{d}^{20° C.}: 1.4463; ¹H NMR (400 MHZ, CDCl₃) δ (ppm): 5.98-5.82 (m, 1H, CH₂CH═CH₂), 5.39-5.20 (m, 2H, CH₂CH═CH₂), 4.64-4.53 (m, 2H, CH₂CH═CH₂), 4.15-3.94 (m, 6H, CH₂O C₆, OCH₂CH₃), 1.83-1.47 (m, 4H, CH₂P C₁, CH₂CH₂O C₅), 1.44-1.19 (m, 12H, CH₂ C₂-C₄, OCH₂CH₃); ¹³C-NMR (101 MHZ, CDCl₃) δ (ppm): 155.15 (s, OC═OO), 131.67 (s, OCH₂CHCH₂), 118.93 (s, OCH₂CHCH₂), 68.41 (s, CH₂CH₂O C₆), 68.05 (s, OCH₂CHCH₂), 61.49 (d, ²J_{CP}=6.4 Hz, OCH₂CH₃), 32.54 (s, CH₂CHO C₅), 30.21 (d, ³J_{CP}=16.8 Hz, CH₂CH₂CH₂P C₃), 25.68 (d, J_{CP}=140.7 Hz, CH₂P C₁), 25.32 (d, ⁴J_{CP}=1.2 Hz, CH₂ C₄), 22.40 (d, ²J_{CP}=5.1 Hz, CH₂CH₂P C₂), 16.55 (d, ³J_{CP}=6.0 Hz, OCH₂CH₃); ³¹P NMR (CDCl₃) δ (ppm): 32.23.

### Step 2: Preparation of 6-(allyloxycarbonyloxy)hexylphosphonic acid (Phn-6C-Allcarb) (S7)

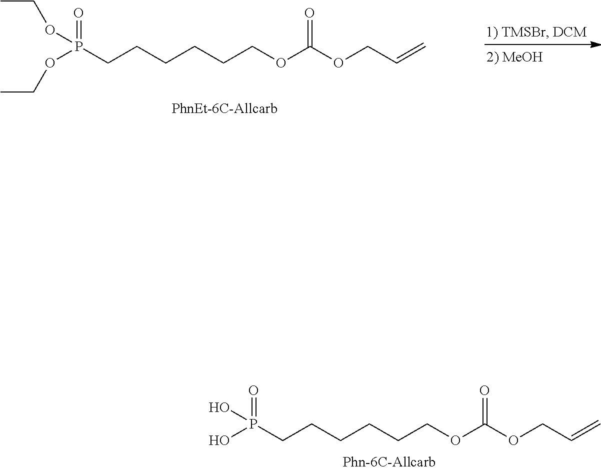 [SMILES: C=CCOC(=O)OCCCCCCP(=O)(OCC)OCC ; C=CCOC(=O)OCCCCCCP(=O)(O)O]

The synthesis was conducted in analogy to Synthesis Example 3, Step 2, from PhnEt-6C-Allcarb (1 eq., 2.1 g, 6.5 mmol) with TMSBr (2.5 eq., 2.2 ml, 16.25 mmol) to isolate 1.5 g (87% of th.) of the desired phosphonic acid Phn-6C-Allcarb as a brown viscous liquid.

¹H NMR (400 MHZ, CDCl₃) δ (ppm): 6.02-5.84 (m, 1H, CH₂CH═CH₂), 5.40-5.21 (m, 2H, CH₂CH═CH₂), 4.67-4.53 (m, 2H, CH₂CH═CH₂), 4.13 (t, J=6.6 Hz, 2H, CH₂O), 1.94-1.56 (m, 6H, CH₂P C₁, CH₂CH₂O C₅, CH₂ C₄), 1.49-1.35 (m, 4H, CH₂CH₂P C₂, CH₂ C₃): 13C-NMR (101 MHZ, CDCl₃) δ (ppm): 155.13 (s, OC═OO), 131.61 (s, OCH₂CHCH₂), 118.92 (s, OCH₂CHCH₂), 68.40 (s, CH₂O C₆), 68.01 (s, OCH₂CHCH₂), 30.02 (d, ³J_{CP}=17.0 Hz, CH₂CH₂CH₂P C₃), 28.52 (s, CH₂CH₂O C₅), 25.45 (d, ¹J_{CP}=141 Hz, CH₂P C₁), 25.31 (s, CH₂ Cc), 21.93 (d, ²J_{CP}=5.0 Hz, PCH₂CH₂ C₂); ³¹P NMR (CDCl₃) δ (ppm): 36.31; HR-MS (MeOH, ESI, m/z): calcd. 238.26, found 238.31 [M].

### Synthesis Example 8—Preparation of 6-(2,3-bis(allyloxycarbonyloxy)propylthio)hexylphosphonic acid (Phn-6C-bisAllcarb) (S8)

### Step 1: Preparation of 6-(2,3-bis(allyloxycarbonyloxy)propylthio)hexylphosphonic acid diethylester (PhnEt-6C-bisAllcarb)

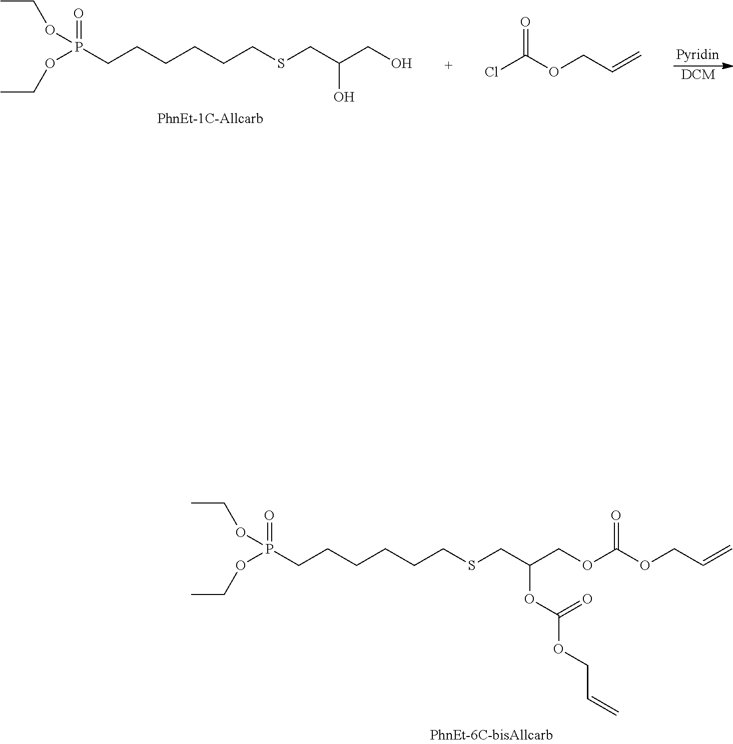

The synthesis was conducted in analogy to Synthesis Example 6, Step 1, from 6-(2,3-dihydroxypropylthio)hexylphosphonic acid diethylester (PhnEt-6C-thioglyc) (1 eq., 3.3 g, 10 mmol) with allyl chloroformic acid ester (4 eq., 4.3 ml, 40 mmol) and pyridine (9 eq., 7.3 ml, 90 mmol). By means of column chromatography (EE), 3.38 g (68% of th.) of the desired phosphonate PhnEt-6C-bisAllcarb were isolated as a brown viscous liquid.

DC (EE) R_{f}=0.4; RI n_{D}^{2° C.}: 1.4774; ¹H NMR (400 MHZ, CDCl₃) δ (ppm): 6.00-5.85 (m, 2H, 2H, 2×CH₂CH═CH₂), 5.41-5.22 (m, 4H, 2×CH₂CH═CH₂), 5.01-4.92 (m, 1H, CHO), 4.67-4.58 (m, 4H, CH₂CH═CH₂), 4.48 (dd, J=11.9, 3.2 Hz, 1H, CH₂OC═O), 4.30 (dd, J=11.9, 5.9 Hz, 1H, CH₂OC═O), 4.18-3.97 (m, 4H, OCH₂CH₃), 2.83-2.69 (m, 2H, SCH₂CH), 2.60-2.52 (m, 2H, CH₂CH₂S C₆), 1.79-1.51 (m, 6H, PCH₂ C₁, PCH₂CH₂ C₂, CH₂CH₂S C₅), 1.38 (m, 4H, CH₂ C₃-C₄), 1.31 (t, J=7.1 Hz, 6H, OCH₂CH₃); ¹³C-NMR (101 MHZ, CDCl₃) δ (ppm): 154.77 (s, OC═OO), 154.42 (s, OC═OO), 131.44 (s, OCH₂CHCH₂), 119.29 (s, OCH₂CHCH₂), 119.21 (s, OCH₂CHCH₂), 74.84 (s, CHO), 68.90 (d, J=3.1 Hz, 2×OCH¿CHCH₂), 66.97 (s, CH₂O), 61.53 (d, ²J_{CP}=6.5 Hz, OCH₂CHs), 32.80 (s, SCH₂CH), 31.95 (s, CH₂CH₂S), 30.27 (d, ³J_{CP}=16.7 Hz, CH₂CH₂CH₂P C₃), 29.33 (s, CH₂CH₂S), 28.34 (d, ⁴J_{CP}=1.3 Hz, CH₂ C₄), 25.77 (d, ¹J_{CP}=140.6 Hz, PCH₂ C₁), 22.47 (d, ²J_{CP}=5.2 Hz, PCH₂CH₂ C₂), 16.61 (d, ³J_{CP}=6.1 Hz, OCH₂CH₃); ³¹P NMR (CDCl₃) 0 (ppm): 32.33.

### Step 2: Preparation of 6-(2,3-bis(allyloxycarbonyloxy)propylthio)hexylphosphonic acid (Phn-6C-bisAllcarb) (S8)

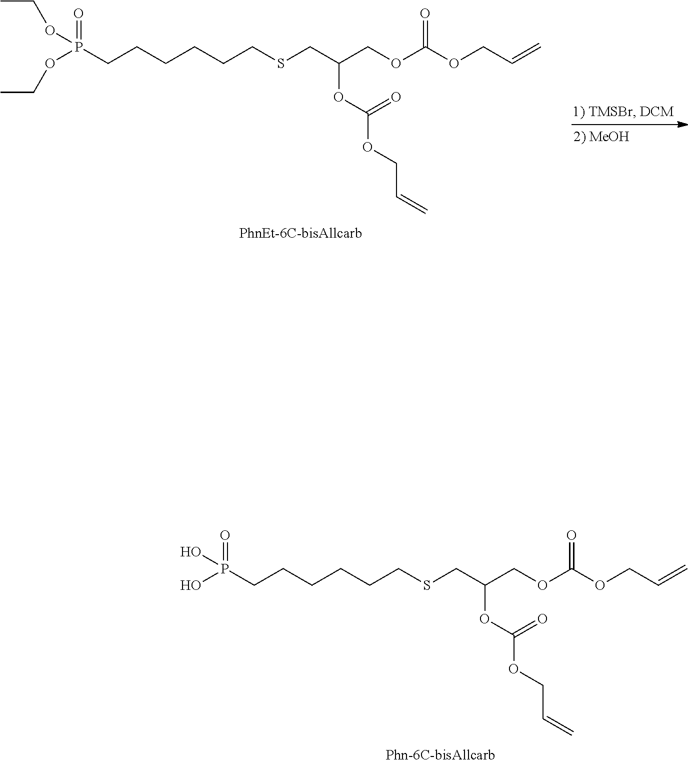 [SMILES: C=CCOC(=O)OCC(CSCCCCCCP(=O)(OCC)OCC)OC(=O)OCC=C ; C=CCOC(=O)OCC(CSCCCCCCP(=O)(O)O)OC(=O)OCC=C]

The synthesis was conducted in analogy to Synthesis Example 3, Step 2, from PhnEt-6C-bisAllcarb (1 eq., 1.9 g, 3.8 mmol) with TMSBr (2.5 eq., 1.3 ml, 9.5 mmol) to isolate 1.61 g (87% of th.) of the desired phosphonic acid Phn-6C-bisAllcarb as a brown viscous liquid.

RI n_{D}^{2° C.}: 1.4877; ¹H NMR (400 MHZ, CDCl₃) δ (ppm): 7.90 (s, 2H, 2×CH₂CH═CH₂), 6.01-5.84 (m, 2H), 5.43-5.23 (m, 4H, 2×CH₂CH═CH₂), 5.04-4.92 (m, 1H, CHO), 4.68-4.58 (m, 4H, 2×CH₂CH═CH₂), 4.48 (dd, J_{HH}=11.9, 3.1 Hz, 1H, CH₂OC═O), 4.31 (dd, J_{HH}=11.9, 6.0 Hz, 1H, CH₂OC═O), 2.80-2.72 (m, 2H, SCH₂CH), 2.62-2.52 (m, 2H, CH₂CH₂S C₈), 1.90-1.73 (m, 2H, PCH₂ C₁), 1.70-1.53 (m, 4H, PCH₂CH₂ C₂, CH₂CH₂S C₅), 1.47-1.35 (m, 4H, CH₂ C₃-C₄); ¹³C-NMR (101 MHZ, CDCl₃) δ (ppm): 154.81 (s, OC═OO), 154.49 (s, OC═OO), 131.44 (s, OCH₂CHCH₂), 119.29 (s, OCH₂CHCH₂), 119.21 (s, OCH₂CHCH₂), 74.84 (s, CHO), 68.97 (d, J=3.1 Hz, 2×OCH₂CHCH₂), 67.03 (s, CH₂O), 32.72 (s, SCH₂CH), 31.94 (s, CH₂CH₂S), 29.90 27 (d, 3J_{CP}=17.1 Hz, CH₂CH₂CH₂P C₃), 29.21 (s, CH₂CH₂S), 28.15 (d, ⁴J_{CP}=1.3 Hz, CH₂ C₄), 25.32 (d, ¹J_{CP}=140.4 Hz, PCH₂ C₁), 21.88 (d, ²J_{CP}=5.1 Hz, PCH₂CH₂ C₂); ³¹P NMR (CDCB) δ (ppm): 37.05; HR-MS (MeOH, ESI, m/z): calcd. 440.44, found 440.41 [M].

### Synthesis Examples 9 and 10—Preparation of Comparative Primers with Vinyl (enyl) as PG and Phosphonic Acid (Phn) as AM

### Synthesis Example 9—Preparation of hex-5-en-1-yl phosphonic acid (Phn-6C-Enyl) (S9)

### Step 1: Preparation of hex-5-en-1-yl phosphonic acid diethylester (PhnEt-6C-Enyl)

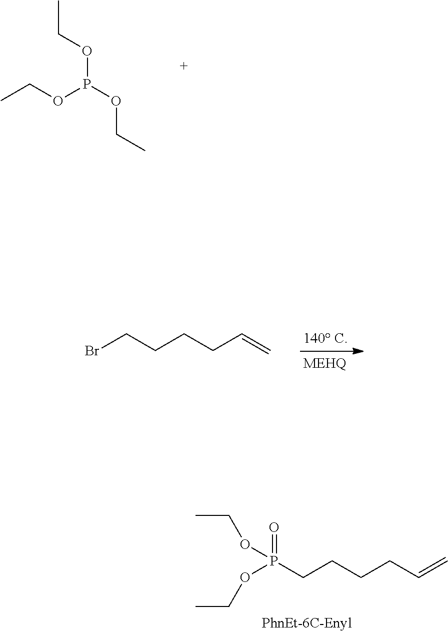

A distillation apparatus provided with a Vigreux column was charged with 9.8 g (1 eq., 60 mmol) of hex-5-en-1-ylbromide, 12.0 g (1.2 eq., 72 mmol) of triethyl phosphite, and 11 mg of p-methoxyphenol (hydroquinone monomethyl ether, MEHQ) under argon atmosphere. The solution was heated to 140° ° C. for 24 h, forming ethyl bromide (b.p. 38° C.) as a byproduct, which was distilled off during the reaction. The reaction progress was monitored by ³¹P NMR. After the reaction was complete, the product was fractionally distilled off under high vacuum (0.011 mbar) at 75° C. to obtain 8.99 g (68% of th.) of the desired phosphonate PhnEt-6C-Enyl as a colorless liquid.

DC (PE/EE 1:2) R_{f}=0.23; RI n_{D}^{2° C.}: 1.4540; ¹H NMR (400 MHZ, CDCl₃) δ (ppm): 5.84-5.69 (m, 1H, CH═CH₂), 5.04-4.89 (m, 2H, CH═CH₂), 4.16-4.00 (m, 4H, OCH₂CH₃), 2.11-2.00 (m, 2H, CH₂CH), 1.78-1.53 (m, 4H, CH₂), 1.51-1.41 (m, 2H, PCH₂), 1.30 (1, ³J_{HH}=7.1 Hz, 6H, OCH₂CH₃); ¹³C-NMR (101 MHZ, CDCl₃) δ (ppm): 138.31 (s, CH═CH₂), 114.92 (s, CH═CH₂), 61.50 (d, J=6.5 Hz, OCH₂CH₃), 33.31 (d, ⁴J_{CP}=1.4 Hz, CH₂CH), 29.88 (d, ³J_{CP}=17.0 Hz, PCH₂CH₂CH₂), 26.72-24.62 (d, ¹J_{CP}=140.6 Hz, PCH₂, 22.03 (d, ²J_{CP}=5.2 Hz, PCH₂CH₂), 16.62 (d, ³J_{CP}=6.1 Hz, OCH₂CH₃); ³¹P NMR (CDCl₃) δ (ppm): 32.31.

### Step 2: Preparation of hex-5-en-1-yl phosphonic acid (Phn-6C-Enyl) (S9)

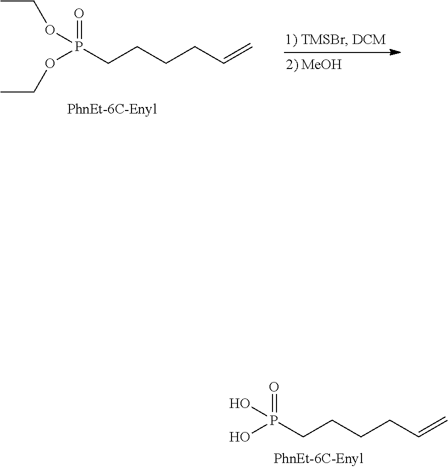

The synthesis was conducted in analogy to Synthesis Example 3, Step 2, from PhnEt-6C-Enyl (1 eq., 3.3 g, 15 mmol) with TMSBr (3 eq., 6 ml, 45 mmol) to isolate 2.51 g (84% of th.) of the desired phosphonic acid Phn-6C-Enyl as a colorless solid.

Fp.: 97.2-100.1° C.: 1H NMR (400 MHZ, CDCl₃) δ (ppm): 9.47 (s, 2H, OH), 5.88-5.67 (m, 1H, CH═CH₂). 5.08-4.89 (m, 2H, CH═CH₂), 2.12-1.99 (m, 2H, CH₂CH), 1.85-1.55 (m, 4H, 2×CH₂), 1.55-1.41 (m, 2H, PCH₂); ¹³C-NMR (101 MHZ, CDCl₃) δ (ppm): 138.17 (s, CH═CH₂), 114.88 (s, CH═CH₂), 33.16 (s, CH₂CH), 29.58 (d, ³J_{CP}=17.0 Hz, PCH₂CH₂CH₂), 26.15-23.79 (d, ¹J_{CP}=140.8 Hz, PCH₂), 21.49 (d, ²J_{CP}=5.0 Hz, PCH₂CH₂); ³¹P NMR (CDCl₃) δ (ppm): 37.41.

### Synthesis Example 10—Preparation of undec-10-en-1-yl phosphonic acid (Phn-11C-Enyl) (S10)

### Step 1: Preparation of undec-10-en-1-yl phosphonic acid diethylester (PhnEt-11C-Enyl)

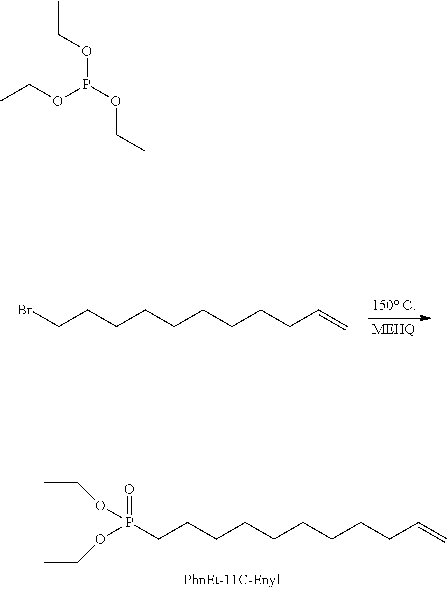

The synthesis was conducted in analogy to Synthesis Example 8, Step 1, from undec-10-en-1-ylbromide (1 eq., 8.2 g, 35 mmol), triethyl phosphite (1.3 eq., 8 ml, 45.5 mmol), and 20 mg of MEHQ. After the reaction was complete, the product was purified by column chromatography (EE) to isolate 6.75 g (66% of th.) of the desired phosphonate PhnEt-11C-Enyl as a yellowish viscous liquid.

DC (EE) R_{f}=0.38; RI n_{D}^{2° C.}: 1.4462; ¹H NMR (400 MHZ, CDCl₃) δ (ppm): 5.86-5.71 (m, 1H, CH═CH₂), 5.01-4.93 (m, 1H, CH═CH₂), 4.93-4.87 (m, 1H, CH═CH₂), 4.16-3.99 (m, 4H, OCH₂CH₃), 2.09-1.94 (m, 2H, CH₂CH═CH₂), 1.78-1.64 (m, 2H, PCH₂), 1.63-1.51 (m, 2H, PCH₂CH₂), 1.41-1.20 (m, 18H, 2×OCH₂CH₃, 6×CH₂ C₃-C₈); ¹³C-NMR (101 MHZ, CDCl₃) δ (ppm): 139.40 (s, CH═CH₂), 114.24 (s, CH═CH₂ C₁₁), 61.49 (d, J=6.4 Hz, OCH₂CH₃), 33.90 (s, CH₂CH), 30.71 (d, ³J_{CP}=17.0 Hz, PCH₂CH₂CH₂ C₃), 29.51 (s, CH₂ C₆), 29.42 (s, CH₂ C₇), 29.19 (s, CH₂ C₅ C₈)), 29.02 (s, CH₂ C₄), 26.66-24.96 (d, ¹J_{CP}=140.4 Hz, PCH₂ C₁), 22.51 (d, ²J_{CP}=5.3 Hz, PCH₂CH₂ C₂), 16.60 (d, ³J_{CP}=6.0 Hz, OCH₂CH₃); ³¹P NMR (CDCl₃) δ (ppm): 32.65.

### Step 2: Preparation of undec-10-en-1-yl phosphonic acid (Phn-11C-Enyl) (S10)

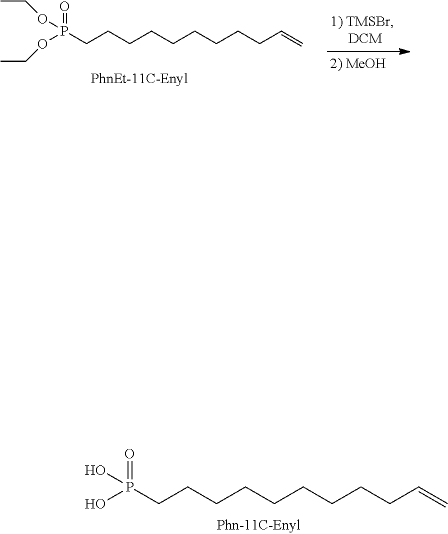 [SMILES: C=CCCCCCCCCCP(=O)(OCC)OCC ; C=CCCCCCCCCCP(=O)(O)O]

The synthesis was conducted in analogy to Synthesis Example 3, Step 2, from PhnEt-11C-Enyl (1 eq., 5.8 g, 20 mmol) with TMSBr (3 eq., 8 ml, 60 mmol) to isolate 4.65 g (99% of th.) of the desired phosphonic acid Phn-11C-Enyl as a colorless solid.

Fp.: 74.2-76.9° C.; ¹H NMR (400 MHZ, CDCl₃) δ (ppm): 5.91-5.70 (m, 1H, CH═CH₂), 5.02-4.96 (m, 1H, CH═CH₂), 4.95-4.91 (m, 1H, CH═CH₂), 2.12-1.95 (m, 2H, CH₂CH═CH₂), 1.81-1.69 (m, 2H, PCH₂), 1.67-1.55 (m, 2H, PCH₂CH₂), 1.46-1.13 (m, 12H, 6×CH₂ C₃-C₆); ¹³C-NMR (101 MHZ, CDCl₃) δ (ppm): 139.24 (s, CH═CH₂), 114.28 (s, CH═CH₂ C₁₁), 33.95 (s, CH₂CH), 30.56 (d, ³J_{CP}=17.1 Hz, PCH₂CH₂CH₂ C₃), 29.56 (s, CH₂ C₆), 29.46 (s, CH₂ C₇), 29.24 (s, CH₂ C₈), 29.17 (s, CH₂ C₅), 29.07 (s, CH₂ C₄), 25.34 (d, ¹J_{CP}=141 Hz, PCH₂ C₁), 22.12 (d, ²J_{CP}=5.0 Hz, PCH₂CH₂ C₂); ³¹P NMR (CDCl₃) δ (ppm): 37.52.

### Synthesis Example 11—Preparation of a Comparative Primer with Vinyl (Enyl) as PG and 4-pyrocatechyl (Catechol) as AM

### 4-Allylpyrocatechol (Catechol-3C-Enyl) (S11)

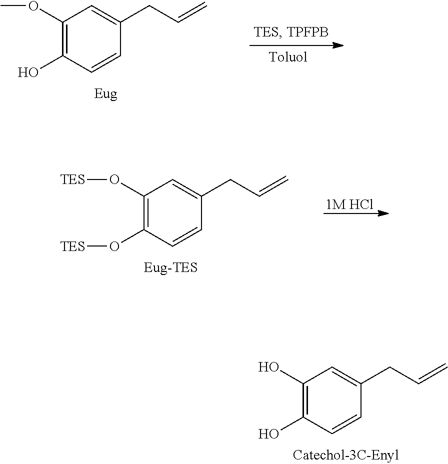

The synthesis known from literature was carried out as described in Donovan et al., RSC Adv. 4, 61927-61935 (2014) from 4-allyl-2-methoxyphenol (Eugenol, Eug) by silylation with triethylsilane (TES) with the cleavage of CH₄ in the presence of tris(pentafluorophenyl)borane (TPFPB), followed by hydrolysis of the double-silylated intermediate Eug-TES with 1 M of aqueous HCl to obtain the deisired Catechol-3C-Enyl.

DC (PE:EE 4:1) R_{f}=0.43; Fp.: 45.1-45.3° C.: ¹H NMR (400 MHZ, MeOD-d) δ (ppm): 6.89-6.53 (m, 3H, Ar—H), 6.03-5.80 (m, 1H, CH═CH₂), 5.13-5.06 (m, 1H, CH═CH₂), 5.02 (1, ²J_{HH}=1.5 Hz, 1H, CH═CH₂). 3.28 (d, ³J_{HH}=6.7 Hz, 2H, CH₂CH═CH₂); ¹³C-NMR (101 MHZ, MeOD-d) δ (ppm): 143.39 (s, Ar—C_{q}—O), 141.66 (s, Ar—C_{q}—O), 137.61 (s, CH═CH₂), 133.31 (s, Ar—C_{q}), 121.05 (s, Ar—C), 115.72 (s, Ar—C), 115.62 (s, CH═CH₂), 115.36 (s, Ar—C), 39.51 (s, CH₂CH═CH₂).

### Examples 1 to 9—Preparation of Primers with Norbornenyl (Norb) as PG and Phosphonic Acid (Phn) or 4-Pyrocatechyl (Catechol) as AM

### Example 1—Preparation of N-(4-hydroxy-4,4-diphosphonobutyl)-5-norbornene-2-carboxylic acid amide (N-(4-hydroxy-4,4-diphosphonobutyl)alendronic acid amide) (Alendronate-Norb) (E1)

### Step 1: Preparation of 5-norbornene-2-carboxylic acid-2,5-dioxopyrrolidine amide (Norb-NHS)

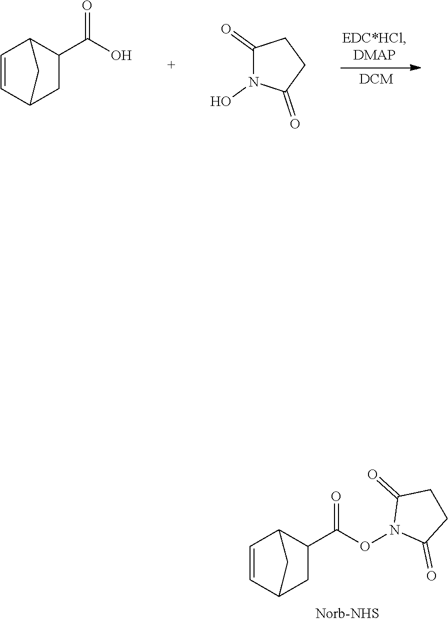 [SMILES: O=C(O)C1CC2C=CC1C2 ; O=C1CCC(=O)N1O ; O=C(ON1C(=O)CCC1=O)C1CC2C=CC1C2]

The synthesis known from literature was carried out as described in Werther et al., Chem. Eur. J. 23(72), 18216-18224 (2017) from 5-norbornene-2-carboxylic acid and 1-hydroxypyrrolidine-2,5-dione in the presence of 1-ethyl-3-(3-dimethylaminopropyl)carbodiimide hydrochloride (EDC·HCl) and 4-dimethylaminopyridine (DMAP) in dry DCM.

DC (EE/PE, 3:1) R_{f}=0.74; Fp.: 87.2-94.0° C.; ¹H NMR (400 MHZ, CDCl₃) δ (ppm): 6.21 (dd, ³J_{HH}=3.0 Hz, ²J_{HH}=5.3 Hz, 1H, CH═CH), 6.12 (dd, ³J_{HH}=3.0 Hz, ²J_{HH}=5.3 Hz, 1H, CH═CH), 3.43-3.35 (m, 1H, CH norb), 3.29-3.19 (m, 1H, CH norb), 2.98 (m, 1H, CHC═O), 2.81 (s, 4H, 2×CH₂), 2.09-1.95 (m, 1H, CH₂ norb), 1.57-1.40 (m, 3H, CH₂ bridge+CH₂ norb), 1.37-1.29 (m, 1H, CH₂ bridge); ¹³C-NMR (101 MHz, CDCl₃) δ (ppm): 171.75 (s, C═O), 170.08 (s, NC═O), 169.43 (s, NC═O), 138.24 (s, CH═CH), 135.38 (s, CH═CH), 49.76 (s, CH₂ bridge endo), 47.24 (s, CH norb exo), 46.52 (s, CH norb endo), 46.50 (s, CH₂ bridge exo), 42.63 (s, CH norb endo), 41.89 (s, CH norb exo), 40.72 (s, CHC═O endo), 40.40 (s, CHC═O exo), 31.08 (s, CH₂ norb exo) 29.68 (s, CH₂ norb endo), 25.72 (s, 2×CH₂).

### Step 2: Preparation of N-(4-hydroxy-4,4-diphosphonobutyl)-5-norbornene-2-carboxylic acid amide sodium salt (Alendronate-Norb) (E1)

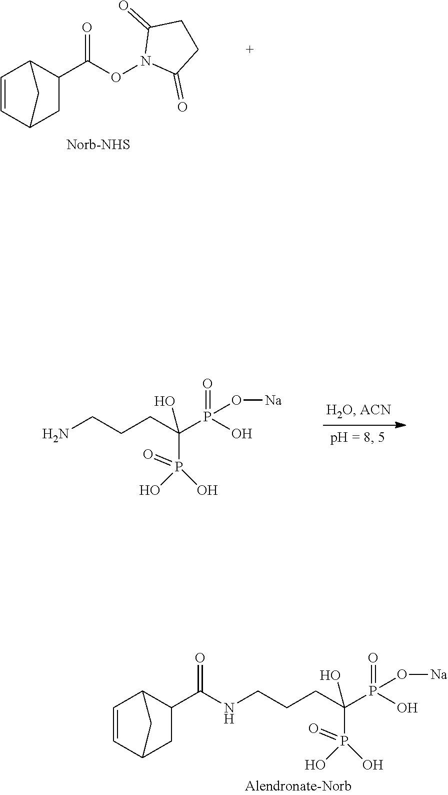

Alendronic acid monosodium salt trihydrate (1 eq., 2.6 g, 8 mmol) was dissolved in 60 ml of deionized water and the pH was adjusted to 8.5 with 1 M of NaOH solution. Norb-NHS (2 eq., 3.8 g, 16 mmol) was dissolved in a second flask in 60 ml of acetonitrile, and the organic solution was added portionwise to the aqueous alendronic acid solution, wherein the pH value was measured after each portion and readjusted to 8.5 when required. After 24 h of stirring at room temperature, the solvents were evaporated, and the raw product was recrystallized from ethanol to isolate 3.5 g (99% of th.) of the desired compound Alendronate-Norb as a colorless solid.

Fp.: 212.3° C. (dec.); ¹H NMR (400 MHz, D₂O) δ (ppm): 6.30-5.93 (m, 2H, CH═CH), 3.21 (t, ³J_{HH}=6.9 Hz, 1H, CH norb), 3.18-3.13 (m, 2H, CH₂NH), 3.03-2.96 (m, 1H, CH norb), 2.95-2.90 (m, 1H, C_{q}OH), 2.67 (s, 3H, 3×POH), 2.49-2.35 (m, 1H, CHC═O), 1.99-1.87 (m, 3H, C_{q}CH₂, CH₂ norb), 1.84-1.71 (m, 2H, CH₂ norb, CH₂ bridge), 1.41-1.35 (m, 1H, CH₂ bridge), 1.35-1.30 (m, 2H, CH₂CH₂NH); ¹³C-NMR (101 MHZ, D₂O) δ (ppm): 178.77 (s, C═O), 138.32 (s, CH═CH), 132.08 (s, CH═CH), 73.88 (t, ¹J_{CP}=132.2 Hz, C_{q}), 49.50 (s, CH₂ bridge endo), 46.43 (m, CH norb endo/exo), 45.97 (s, CH₂ bridge exo), 44.12 (s, CH norb endo), 43.97 (s, CH norb exo), 42.56 (s, CHC═O endo), 41.30 (s, CHC═O exo), 40.04 (s, CqCH₂CH₂CH₂NH), 31.19 (s, CqCH₂CH₂CH₂NH), 29.12 (s, CH₂ norb exo), 28.75 (s, CH₂ norb endo), 23.60 (t, ³J_{CP}=6.4 Hz, CqCH₂CH₂CH₂NH); ³¹P NMR (D₂O) δ (ppm): 18.31; elementary analysis: calcd. for C₁₂H₂₀NNaO₈P₂: C-36.84, H-5.15, N-3.58, Na-5.88, O-32.72, P-15.83; found: C-36.85, H-4.86, N-3.62, Na-5.01, O-33.32, P-15.79.

### Example 2—Preparation of N-[2-(3,4-dihydroxyphenyl)ethyl]-5-norbornene-2-carboxylic acid amide (Catechol-2C-Norb) (E2)

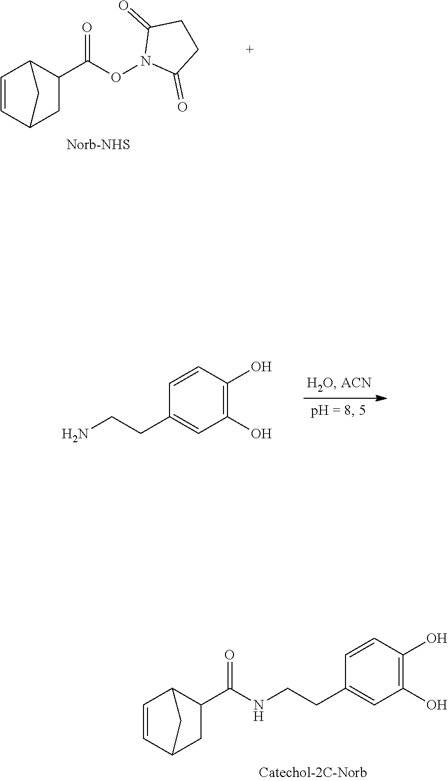 [SMILES: O=C(ON1C(=O)CCC1=O)C1CC2C=CC1C2 ; NCCc1ccc(O)c(O)c1 ; O=C(NCCc1ccc(O)c(O)c1)C1CC2C=CC1C2]

The synthesis was conducted in analogy to Synthesis Example 12, Step 2, from Norb-NHS (1.5 eq., 2.3 g, 10 mmol) and dopamine hydrochloride (1 eq., 2.4 g, 15 mmol) in a water/acetonitrile mixture at pH 8.5. The raw product obtained by evaporating the solvent was purified by column chromatography (ethyl acetate:petroleum ether, EE:PE, 1:1-3:1) to obtain 1.4 g (60% of th.) of the desired compound Catechol-2C-Norb as a colorless viscous liquid.

DC (EE:PE, 3:1) R_{f}=0.42: ¹H NMR (400 MHZ, CDCl₃) δ (ppm): 6.82 (d, ³J_{HH}=8.0 Hz, 1H, CH Ar), 6.73 (d, ³J_{HH}=2.0 Hz, 1H, CH Ar), 6.54 (dd. ³J_{HH}=8.1 Hz, 4J_{HH}=2.0 Hz, 1H, CH Ar), 6.17 (dd, ³J_{HH}=5.7, ⁴J_{HH}=3.1 Hz, 1H, CH═CH), 5.82 (dd, ³J_{HH}=5.7, ⁴J_{HH}==2.8 Hz, 1H, CH═CH), 5.63 (1, ³J_{HNCH}=5.8 Hz, 1H, NH), 3.42 (q, ³J_{HH}=6.7 Hz, 2H, CH₂NH), 3.06 (s, 1H, CH norb), 2.95-2.76 (m, 2H, CH norb, CHC═O norb), 2.64 (t, ³J_{HH}=7.0 Hz, 2H, C_{Ar}CH₂), 2.03-1.82 (m, 1H, CH norb), 1.50-1.34 (m, 1H, CH₂ bridge), 1.34-1.09 (m, 1H, CH₂ bridge); ¹³C-NMR (101 MHZ, CDCl₃) δ (ppm): 176.47 (s, C═O), 145.04 (s, C—OH), 143.39 (s, C—OH), 138.31 (s, CH═CH), 132.26 (s, CH═CH), 129.83 (s, C_{Ar}CH₂), 120.50 (s, CH Ar), 115.70 (s, CH Ar), 115.41 (s, CH Ar), 50.22 (s, CH₂ bridge), 46.42 (s, CH norb), 45.11 (s, CH norb), 42.89 (CHC═O norb), 41.10 (s, CH₂NH), 35.67 (s, C_{Ar}CH₂), 30.43 (s, CH₂ norb); elementary analysis: calcd. for C₁₆H₁₉NO₃. C-70.31, H-7.01, N-5.12, O-17.56; found: C-70.58, H-7.05, N-4.91, O-18.35.

### Example 3—Preparation of 5-norbornen-2-ylcarbonyloxymethylphosphonic acid (Phn-1C-Norb) (E3)

### Step 1: Preparation of 5-norbornene-2-carboxylic acid chloride (Norb-Cl)

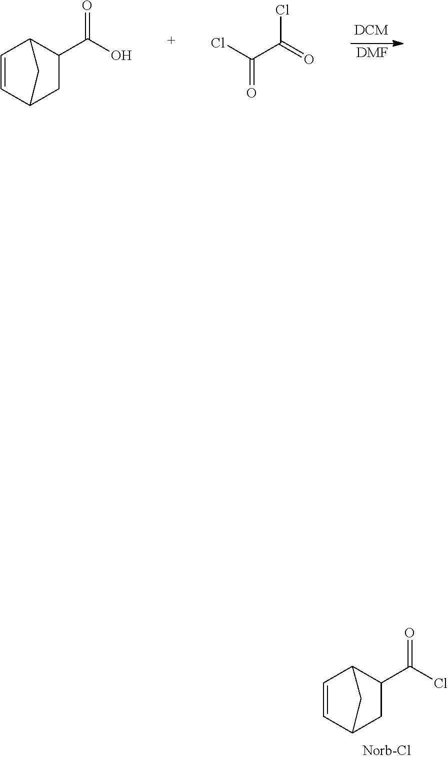

The synthesis known from literature was carried out as described in Chae et al., Macromolecules 51, 3458-3466 (2018) from 5-norbornene-2-carboxylic acid and oxalyl chloride in dry DCM in the presence of catalytic amounts of dimethyl formamide (DMF) to obtain the desired Norb-Cl by distillation as a colorless liquid.

1H NMR (400 MHZ, CDCl₃) δ (ppm): 6.32-6.17 (m, 1H, CH═CH endo/exo), 6.17-5.99 (m, 1H, CH═CH exo/endo), 3.49-2.71 (m, 3H, CH), 2.07-1.87 (m, 1H, CH₂, CH₂ bridge), 1.57-1.38 (m, 2H, CH₂, CH₂ bridge), 1.37-1.28 (m, 1H, CH₂, CHąbridge); ¹³C-NMR (101 MHZ, CDCB) δ (ppm): 175.19 (s, C═O). 139.17 (s, CH═CH exo), 138.83 (s, CH═CH endo), 135.02 (s, CH═CH exo), 131.75 (s, CH═CH endo). 56.56 (s, CHC═O endo), 56.45 (s, CHC═O exo), 49.36 (s, CH₂ bridge), 47.29 (s, CH endo), 47.03 (s, CH exo), 46.43 (s, CH₂ bridge), 43.00 (s, CH endo), 42.00 (s, CH exo), 31.29 (s, CH₂ exo), 30.21 (s, CH₂ endo).

### Step 2: Preparation of 5-norbornen-2-ylcarbonyloxymethylphosphonic acid diethylester (PhnEt-1C-Norb)

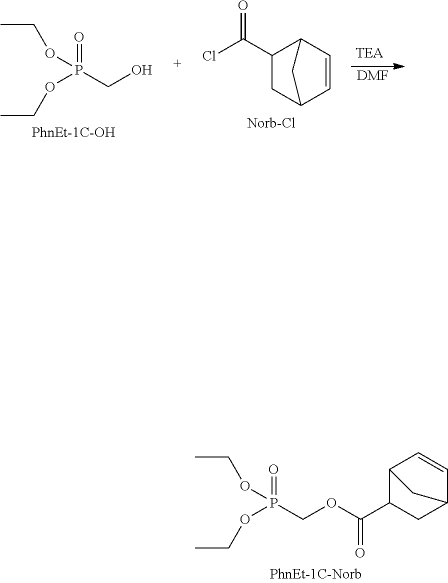

Phosphonic acid diethyl(hydroxymethyl)ester PhnEt-1C—OH (1 eq., 2.2 ml, 15 mmol) was slowly (within 15 min) added to a mixture of Norb-Cl (1.5 eq., 3.5 g, 22.5 mmol) and triethylamine (TEA) (1.8 eq., 3.7 ml, 27 mmol) in 50 ml of dry DCM with stirring, then the reaction mixture was stirred at 0° C. for 1 h and at room temperature for 24 h. The solvent and the non-reacted educts were evaporated, and the raw product was again dissolved in 50 ml of DCM. The solution was washed with brine (3×25 ml), and the organic phase was dried over MgSO₄. The solvent was evaporated, and the product was dried under vacuum to constant weight to obtain 4.3 g (99% of th.) of the desired PhnEt-1C-Norb as a brown viscous liquid.

DC (EE) R_{f}=0.53; ¹H NMR (400 MHZ, CDCl₃) δ (ppm): 6.26-5.90 (m, 2H, CH═CH), 4.47-4.23 (m, 2H, PCH₂O), 4.22-4.10 (m, 4H, OCH₂CH₃), 3.27-2.84 (m, 3H, 2×CH norb, CHC═O), 2.00-1.87 (m, 1H, CH₂ norb), 1.57-1.23 (m, 9H, CH₂ norb, CH₂ bridge, 2×OCH₂CH₃); ¹³C-NMR (101 MHZ, CDCl₃) δ (ppm): 175.47 (s, exo C═O), 173.89 (s, endo C═O), 138.34 (s, exo CH═CH), 137.99 (s, endo CH═CH), 135.69 (s, exo CH═CH), 132.42 (s, endo CH═CH), 62.89-62.73 (m, OCH₂CH₃), 56.85 (d, ¹J_{CP}=168.5 Hz, CH₂P C₁ endo), 56.61 (d, ¹J_{CP}=168.7 Hz, CH₂P C₁ exo), 49.73 (s, CH₂ bridge), 46.75 (s, exo CH norb), 46.44 (s, CH₂ bridge), 45.89 (s, endo CH norb), 43.15 (s, endo CH norb), 42.93 (s, exo CH norb), 42.63 (s, endo CH norb), 41.77 (s, exo CH norb), 30.57 (s, exo CH₂ norb), 29.51 (s, endo CH₂ norb), 16.53 (d, ³J_{CP}=5.9 Hz, OCH₂CH₃); ³¹P NMR (CDCl₃) δ (ppm): 19.47; HR-MS (MeOH, ESI, m/z): calcd. 288.28, found 288.41 [M].

### Step 3: Preparation of 5-norbornen-2-ylcarbonyloxymethylphosphonic acid (Phn-1C-Norb) (E3)

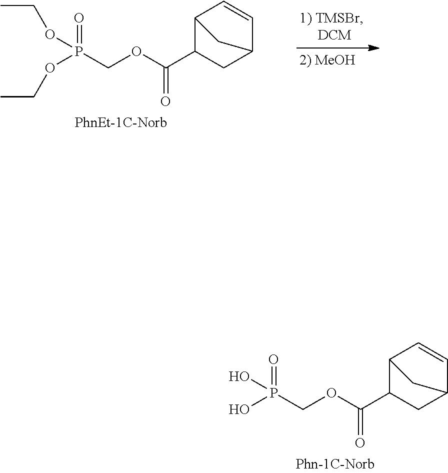 [SMILES: CCOP(=O)(COC(=O)C1CC2C=CC1C2)OCC ; O=C(OCP(=O)(O)O)C1CC2C=CC1C2]

The synthesis was conducted in analogy to Synthesis Example 3, Step 2, from PhnEt-1C-Norb (1 eq., 5.2 g, 18 mmol) with TMSBr (3 eq., 6 ml, 45 mmol) to isolate 4.17 g (99% of th.) of the desired phosphonic acid Phn-1C-Norb as a brown viscous liquid. ¹H NMR (400 MHZ, CDCl₃) δ (ppm): 8.17 (s, 2H), 6.23-5.87 (m, 2H, CH═CH), 4.51-4.27 (m, 2H, PCH₂O), 3.30-3.14 (m, 1H, CH norb), 3.12-2.99 (m, 1H, CH norb), 2.97-2.87 (m, 1H, CHC═O), 2.01-1.86 (m, 1H, CH₂ norb), 1.62-1.19 (m, 3H, CH₂ norb, CH₂ bridge); ¹³C-NMR (101 MHZ, CDCl₃) δ (ppm): 176.13 (s, exo C═O), 174.86 (s, endo C═O), 138.34 (s, exo CH═CH), 137.99 (s, endo CH═CH), 135.69 (s, exo CH═CH), 132.42 (s, endo CH═CH), 57.63 (d, ¹J_{CP}=169.3 Hz, CH₂P C₁ exo), 56.96 (d, ¹J_{CP}=169.2 Hz, CH₂P C₁ endo), 49.78 (s, CH₂ bridge), 46.80 (s, exo CH norb), 46.48 (s, CH₂ bridge), 46.03 (s, endo CH norb), 43.20 (s, endo CH norb), 42.99 (s, exo CH norb), 42.72 (s, endo CH norb), 41.79 (s, exo CH norb), 30.61 (s, exo CH₂ norb), 29.51 (s, endo CH₂ norb); ³¹P NMR (CDCl₃) δ (ppm): 20.93; HR-MS (MeOH, ESI, m/z): calcd. 288.28, found 288.28 [M].

### Example 4—Preparation of 6-(5-Norbornen-2-ylcarbonyloxy)hexylphosphonic acid (Phn-6C-Norb) (E4)

Step 1: Preparation of 6-(5-norbornen-2-ylcarbonyloxy)hexylphosphonic acid diethylester (PhnEt-6C-Norb)

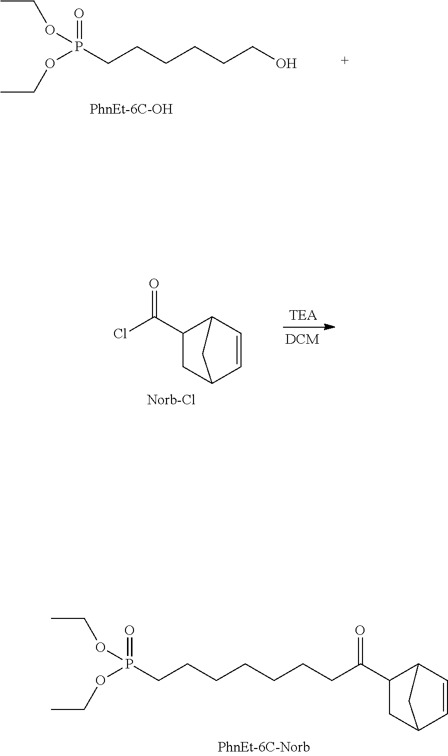 [SMILES: CCOP(=O)(CCCCCCO)OCC ; O=C(Cl)C1CC2C=CC1C2 ; CCOP(=O)(CCCCCCCC(=O)C1CC2C=CC1C2)OCC]

The synthesis was conducted in analogy to Example 3, Step 2, from phosphonic acid diethyl(6-hydroxyhexyl)ester PhnEt-6C—OH (1 eq., 2.9 g, 12 mmol) with Norb-Cl (2.5 eq., 4.7 g, 30 mmol) and TEA (2.8 eq., 4.7 ml, 33 mmol) in DCM to isolate, by column chromatography (PE:EE, 1:1), 3.01 g (70% of th.) of the desired ester PhnEt-6C-Norb as a brown viscous liquid.

DC (EE/MeOH, 19:1) R_{f}=0.5; ¹H NMR (400 MHZ, CDCl₃) δ (ppm): 6.21-5.84 (m, 2H, CH═CH), 4.15-3.90 (m, 6H, OCH₂CH₃, CH₂OC═O), 3.21-3.13+3.04-2.98 (m, 1H, CH norb), 2.95-2.81+2.25-2.13 (m, 2H, CH norb, CHC═O), 1.94-1.79 (m, 1H, CH₂ norb), 1.77-1.52 (m, 5H, CH₂ norb, CH₂ bridge, CH₂P), 1.44-1.20 (m, 14H, 2×OCH₂CH₃, 4×CH₂ alkyl); ¹³C-NMR (101 MHZ, CDCl₃) δ (ppm): 176.37 (exo C═O), 174.87 (endo C═O), 138.12 (s, exo CH═CH), 137.83 (s, endo CH═CH), 135.85 (s, exo CH═CH), 132.42 (s, endo CH═CH), 64.28 (d, J=24.4 Hz, CH₂OC═O), 61.50 (d, ²J_{CP}=6.5 Hz, OCH₂CH₃), 49.71 (s, CH₂ bridge), 46.69 (s, exo CH norb), 46.44 (s, CH₂ bridge), 45.80 (s, endo CH norb), 43.44 (s, endo CH norb), 43.28 (s, exo CH norb), 42.62 (s, endo CH norb), 41.71 (s, exo CH norb), 30.40 (s, exo CH₂ norb), 30.27 (d, ³J_{CP}=17.0 Hz, CH₂CH₂CH₂P C₃), 29.27 (s, CH₂CH₂O C₅), 28.53 (s, endo CH₂ norb), 25.72 (d, ¹J_{CP}=140.8 Hz, CH₂P C₁), 25.60 (d, ⁴J_{CP}=1.4 Hz, CH₂ C₄), 22.45 (d, ²J_{CP}=5.1 Hz, PCH₂CH₂ C₂). 16.57 (d, ³J_{CP}=5.9 Hz, OCH₂CH₃); ³¹P NMR (CDCl₃) δ (ppm): 32.29.

### Step 2: Preparation of 6-(5-Norbornen-2-ylcarbonyloxy)hexylphosphonic acid (Phn6C-Norb) (E4)

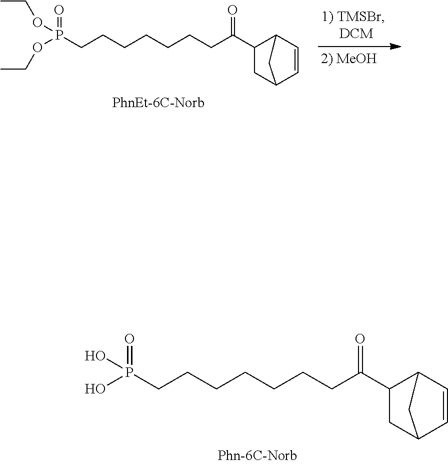 [SMILES: CCOP(=O)(CCCCCCCC(=O)C1CC2C=CC1C2)OCC ; O=C(CCCCCCCP(=O)(O)O)C1CC2C=CC1C2]

The synthesis was conducted in analogy to Synthesis Example 3, Step 2, from PhnEt-6C-Norb (1 eq., 2.9 g, 8 mmol) with TMSBr (2 eq., 2.1 ml, 16 mmol) to isolate 2.37 g (99% of th.) of the desired phosphonic acid Phn-6C-Norb as a brown viscous liquid. 1H NMR (400 MHZ, CDCl₃) δ (ppm): 8.27 (s, 2H, OH), 6.24-5.84 (m, 2H, CH═CH), 4.12-3.95 (m, 2H, CH₂OC═O alkyl), 3.24-3.14 (m, 1H, CH norb), 3.07-2.84 (m, 2H, CH norb, CHC═O), 2.25-2.15 (m, 1H, CH₂ norb), 1.95-1.11 (m, 13H, CH₂ norb, CH₂ bridge, CH₂P, 4×CH₂ alkyl); ¹³C-NMR (101 MHZ, CDCl₃) δ (ppm): 176.60 (s, exo C═O), 175.15 (s, endo C═O), 138.17 (s, exo CH═CH), 137.92 (s, endo CH═CH), 135.90 (s, exo CH═CH), 132.46 (s, endo CH═CH), 64.39 (d, J=24.3 Hz, CH₂OC═O), 49.77 (s, CH₂ bridge), 46.75 (s, exo CH norb), 46.51 (s, CH₂ bridge), 45.87 (s, endo CH norb), 43.52 (s, endo CH norb), 43.35 (s, exo CH norb), 42.67 (s, endo CH norb), 41.77 (s, exo CH norb), 30.48 (s, exo CH₂ norb). 30.04 (d, ³J_{CP}=17.0 Hz, CH₂CH₂CH₂P), 29.36 (s, CH₂CH₂O C₅), 28.51 (s, endo CH₂ norb), 25.54 (s, CH₂ C₄), 25.43 (d, ¹J_{CP}=141 Hz, CH₂P C₁), 22.07 (d, ²J_{CP}=5.0 Hz, PCH₂CH₂ C₂); ³¹P NMR (CDC) δ (ppm): 36.87; HR-MS (MeOH, ESI, m/z): calcd. 302.35, found 302.35 [M].

### Example 5—Preparation of 6-(2,3-bis(5-norbornen-2-ylcarbonyloxy)propylthio)hexylphosphonic acid (Phn-6C-bisNorb) (E5)

### Step 1: Preparation of 6-(2,3-dihydroxypropylthio)hexylphosphonic acid diethylester (PhnEt-6C-thioglyc)

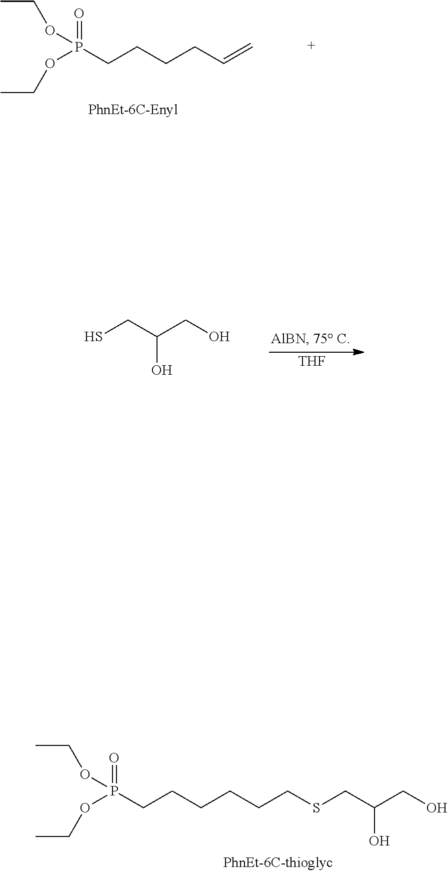 [SMILES: C=CCCCCP(=O)(OCC)OCC ; OCC(O)CS ; "THF" ; CCOP(=O)(CCCCCCSCC(O)CO)OCC]

The synthesis known from literature was carried out as described in Catel et al., Macromol. Mater. Eng. 300, 1010-1022 (2015) from hex-5-en-1-yl phosphonic acid diethylester (PhnEt-6C-Enyl) from Synthesis Example 9, Step 1, by addition of thioglycol in the presence of azobis(isobutyronitrile) (AlBN) in THF.

DC (EE/MeOH, 9:1) R_{f}=0.9; RI n_{D}^{20° C.}: 1.4880; ¹H NMR (400 MHZ, CDCl₃) δ (ppm): 4.17-4.00 (m, 4H, OCH₂CH₃), 3.83-3.68 (m, 2H, CHOH, CH₂OH), 3.63-3.54 (m, 1H, CH₂OH), 2.71 (dd, ²J_{HH}=13.7, ³J_{HH}=5.0 Hz, 1H, SCH₂CH), 2.62 (dd, ²J_{HH}=13.7, ³J_{HH}=7.8 Hz, 1H, SCH₂CH), 2.56 (t, ³J_{HH}=7.4 Hz, 2H, CH₂CH₂S), 1.81-1.68 (m, 2H, PCH₂), 1.68-1.54 (m, 4H, PCH₂CH₂, CH₂CH₂CH₂S), 1.48-1.39 (m, 4H, PCH₂CH₂CH₂, CH₂CH₂S), 1.33 (t, ³J_{HH}=7.1 Hz, 6H, OCH₂CH₃); ¹³C-NMR (101 MHZ, CDCl₃) δ (ppm): 70.32 (s, CHOH), 65.54 (s, CH₂OH), 61.75 (d, ³J_{CP}=6.7 Hz, OCH₂CH₃), 35.78 (S. SCH₂CH), 32.30 (s, CH₂CH₂S), 29.95 (d, ³J_{CP}=16.8 Hz, PCH₂CH₂CH₂), 29.30 (s, CH₂CH₂CH₂S), 28.11 (s, CH₂CH₂S), 26.64-24.44 (d, ¹J_{CP}=140.8 Hz, PCH₂), 22.21 (d, ²J_{CP}=5.1 Hz, PCH₂CH₂), 16.51 (d, ³J_{CP}=6.1 Hz, OCH₂CH₃); ³¹P NMR (CDCl₃) δ (ppm): 32.60.

### Step 2: Preparation of 6-(2,3-bis(5-norbornen-2-ylcarbonyloxy)propylthio)hexylphosphonic acid diethylester (PhnEt-6C-bisNorb)

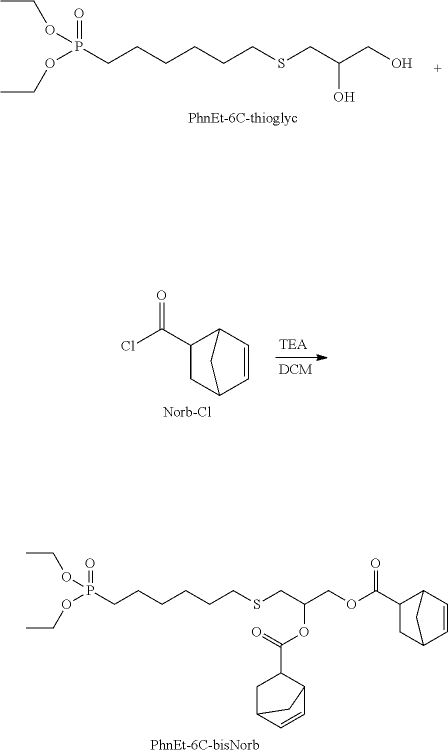 [SMILES: CCOP(=O)(CCCCCCSCC(O)CO)OCC ; O=C(Cl)C1CC2C=CC1C2 ; CCOP(=O)(CCCCCCSCC(COC(=O)C1CC2C=CC1C2)OC(=O)C1CC2C=CC1C2)OCC]

The synthesis was conducted in analogy to Example 3, Step 2, from PhnEt-6C-thioglyc (1 eq., 3.3 g, 10 mmol) with Norb-Cl (2.3 eq., 3.6 g, 23 mmol) and TEA (2.5 eq., 3.5 ml, 25 mmol) in dry DCM to isolate, by column chromatography (EE), 3.0 g (73% of th.) of the desired ester PhnEt-6C-bisNorb as a red viscous liquid.

DC (EE) R_{f}=0.37; ¹H NMR (400 MHZ, CDCl₃) δ (ppm): 6.24-5.87 (m, 4H, 2×CH═CH), 5.23-4.98 (m, 1H, CHO), 4.46-4.15 (m, 2H, CH₂O), 4.09 (m, 4H, OCH₂CH₃), 3.25-3.16 (m, 2H, 2×CH norb), 3.12-2.87 (m, 4H, 2×CH norb, SCH₂CH), 2.74-2.62 (m, 2H, CH₂SCH₂ C₅), 2.60-2.49 (m, 2H, CHC═O), 1.99-1.84 (m, 2H, CH₂ norb), 1.79-1.22 (m, 22H, 2×CH₂ bridge, 5×CH₂ C₁-C₅, CH₂ norb, 2×OCH₂CH₃); ¹³C-NMR (101 MHz, CDCl₃) δ (ppm): 174.14 (m, 2×C═O), 138.14 (s, CH═CH), 137.80 (s, CH═CH), 135.67 (s, CH═CH), 132.39 (s, CH═CH), 70.55-70.34 (m, CHO), 64.48-62.74 (m, CHCH₂O), 61.42 (d, J=6.5 Hz, OCH₂CH₃), 49.64 (s, CH₂ bridge), 46.35 (s, CH₂ norb), 45.82-45.70 (m, CH norb), 43.42-43.25 (m, CHC═O), 42.59-42.49 (m, CH norb), 32.58 (s, SCH₂CH), 32.34 (s, CH₂SCH₂), 30.18 (d, ³J_{CP}=17.0 Hz, PCH₂CH₂CH₂), 29.24 (s, CH₂CH₂CH₂S), 28.26 (s, CH₂CH₂S), 25.65 (d, ¹J_{CP}=140.7 Hz, PCH₂), 22.36 (d, ²J_{CP}=5.2 Hz, PCH₂CH₂), 16.52 (d, ³J_{CP}=6.0 Hz, OCH₂CH₃); ³¹P NMR (CDCl₃) δ (ppm): 32.34.

### Step 3: Preparation of 6-(2,3-bis(5-norbornen-2-ylcarbonyloxy)propylthio)hexylphosphonic acid (Phn-6C-bisNorb) (E5)

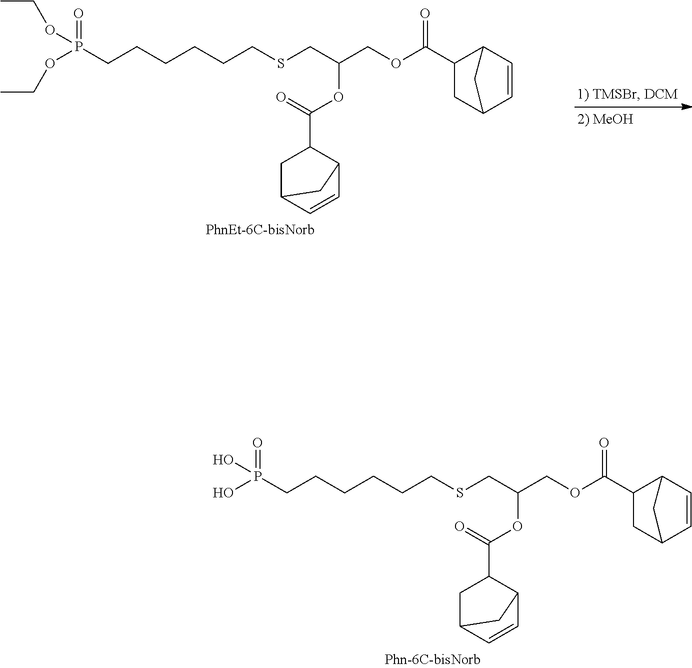 [SMILES: CCOP(=O)(CCCCCCSCC(COC(=O)C1CC2C=CC1C2)OC(=O)C1CC2C=CC1C2)OCC ; O=C(OCC(CSCCCCCCP(=O)(O)O)OC(=O)C1CC2C=CC1C2)C1CC2C=CC1C2]

The synthesis was conducted in analogy to Synthesis Example 3, Step 2, from PhnEt-6C-bisNorb (1 eq., 2.3 g, 4 mmol) with TMSBr (2.3 eq., 1.2 ml, 9.2 mmol) to isolate 1.86 g (91% of th.) of the desired phosphonic acid Phn-6C-bisNorb as a brown viscous liquid.

¹H NMR (400 MHZ, CDCl₃) δ (ppm): 6.24-5.86 (m, 4H, 2×CH═CH), 5.25-4.98 (m, 1H, CHO), 4.46-4.08 (m, 2H, CH₂O), 3.26-3.14 (m, 2H, 2×CH norb), 3.12-2.87 (m, 4H, 2×CH norb, SCH₂ CH), 2.77-2.63 (m, 2H, CH₂SCH₂ C₆), 2.60-2.50 (m, 2H, CHC═O), CH₂ norb), 1.99-1.21 (m, 18H, 2×CH₂ bridge, 5×CH₂ C₁-C₅, 2×CH₂ norb); ¹³C-NMR (101 MHZ, CDCl₃) δ (ppm): 175.99-174.17 (m, 2×C═O), 138.28 (s, CH═CH), 137.99 (s, CH═CH), 135.79 (s, CH═CH), 132.47 (s, CH═CH), 70.80-70.39 (m, CHO), 64.19-63.51 (m, CHCH₂O), 50.78 (s, CH₂ norb), 49.74 (s, CH₂ bridge), 45.82-45.70 (m, CH norb), 43.42-43.25 (m, CHC═O), 42.59-42.49 (m, CH norb), 32.64 (s, SCH₂CH), 32.41 (s, CH₂SCH₂), 30.18 (d, ³J_{CP}=17.0 Hz, PCH₂CH₂CH₂), 29.34 (s, CH₂CH₂CH₂S), 28.25 (s, CH₂CH₂S), 25.58 (s, ¹J_{CP}=140.9 Hz, PCH₂, 22.08 (d, ²J_{CP}=5.2 Hz, PCH₂CH₂); ³¹P NMR (CDCl₃) δ (ppm): 36.46; HR-MS (MeOH, ESI, m/z): calcd. 512.62, found 512.62 [M].

### Example 6—Preparation of 6-(2,3-bis(5-norbornen-2-ylcarbonyloxy)propylthio)hexane-1,1-diyl-bis(phosphonic acid) (bisPhn-6C-bisNorb) (E6)

Step 1: Preparation of 6-(2,3-dihydroxypropylthio)hexane-1,1-diyl-bis(phosphonic acid diethylester) (bisPhnEt-6C-thioglyc)

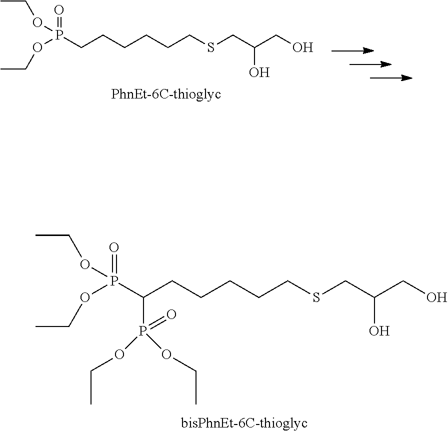 [SMILES: CCOP(=O)(CCCCCCSCC(O)CO)OCC ; CCOP(=O)(OCC)C(CCCCCSCC(O)CO)P(=O)(OCC)OCC]

The synthesis known from literature was carried out as described in Catel et al., Macromol. Mater. Eng. 300, 1010-1022 (2015) from 6-(2,3-dihydroxypropylthio)hexylphosphonic acid diethylester (PhnEt-6C-thioglyc) by protecting the diol with 2,2-dimethoxypropane, then a second phosphonic acid diethylester group was introduced via diethyl chlorophosphite, followed by deprotection of the diol.

DC (EE) R_{f}=0.18; ¹H NMR (400 MHz, CD₃OD) δ (ppm): 4.23-4.11 (m, 8H, 4×OCH₂ CH₃), 3.75-3.67 (m, 1H, CHO), 3.59 (dd, ³J_{HH}=4.7 Hz, ²J_{HH}=11.2 Hz, 1H, CHCH₂ O), 3.53 (dd, ³J_{HH}=5.8 Hz, ²J_{HH}=11.2 Hz, 1H, CHCH₂O), 2.71 (dd, ³J_{HH}=5.7 Hz, ²J_{HH}=13.5 Hz, 1H, SCH₂CH), 2.64-2.45 (m, 4H, P₂CH C₁+ CH₂SCH₂ C₆+SCH₂CH), 1.96-1.84 (m, 2H, CH₂ C₂), 1.61 (m, 4H, CH₂CH₂S C₅, CH₂ C₄), 1.48-1.40 (m, 2H, CH₂ C₃), 1.35 (t, ³J_{HH}=7.1 Hz, 12H, 4×OCH₂CHa); ¹³C-NMR (101 MHZ, CD:OD) δ (ppm): 72.86 (s, CHCH₂O), 65.96 (s, CHCH₂O), 64.18 (d, ²J_{CP}=6.7 Hz, 4×OCH₂CH₃), 36.28 (1, ¹J_{CP}=133.3 Hz, P₂CH C₁), 33.37 (s, SCH₂CH), 30.31 (s, CH₂SCH₂ C₆), 29.38 (s, CH₂CH₂CH₂S C₄), 25.39 (t, ²J_{CP}=5.0 Hz, P₂CHCH₂ C₂), 16.74 (d, ³J_{CP}=6.0 Hz, 2×OCH₂CH₂), 16.70 (d, ³J_{CP}=6.0 Hz, 2×OCH₂CH₃); ³¹P NMR (CD₃OD) δ (ppm): 24.07.

### Step 2: Preparation of 6-(2,3-bis(5-norbornen-2-ylcarbonyloxy)propylthio)hexane-1,1-diyl-bis(phosphonic acid diethylester) (bisPhnEt-6C-bisNorb)

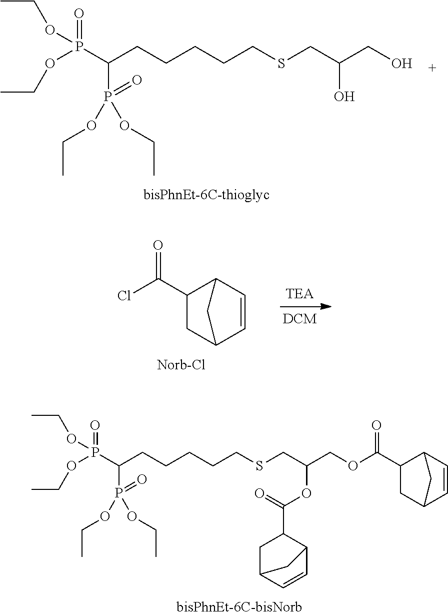 [SMILES: CCOP(=O)(OCC)C(CCCCCSCC(O)CO)P(=O)(OCC)OCC ; O=C(Cl)C1CC2C=CC1C2 ; CCOP(=O)(OCC)C(CCCCCSCC(COC(=O)C1CC2C=CC1C2)OC(=O)C1CC2C=CC1C2)P(=O)(OCC)OCC]

The synthesis was conducted in analogy to Example 3, Step 2, from bisPhn-6C-thioglyc (1 eq., 9.9 g, 22 mmol) with Norb-Cl (2.5 eq., 8.6 g, 55 mmol) and TEA (2.8 eq., 6.2 g, 61.6 mmol) in dry DCM to isolate, by column chromatography (EE), 11.24 g (73% of th.) of the desired ester bisPhnEt-6C-bisNorb as a red viscous liquid.

DC (EE) R_{f}=0.53; ¹H NMR (400 MHZ, CDCl₃) δ (ppm): 6.26-5.90 (m, 2H, CH═CH), 4.47-4.23 (m, 2H, PCH₂O), 4.22-4.10 (m, 4H, OCH₂CH₃), 3.27-2.84 (m, 3H, 2×CH norb, CHC═O), 2.00-1.87 (m, 1H, CH₂ norb), 1.57-1.23 (m, 9H, CH₂ norb, CH₂ bridge, 2×OCH₂CH₃); ¹³C-NMR (101 MHz, CDCl₃) δ (ppm): 175.47 (s, exo C═O), 173.89 (s, endo C═O), 138.34 (s, exo CH═CH), 137.99 (s, endo CH═CH), 135.69 (s, exo CH═CH), 132.42 (s, endo CH═CH), 62.89-62.73 (m, OCH₂CH₃), 56.85 (d, ¹J_{CP}=168.5 Hz, CH₂P C₁ endo), 56.61 (d, ¹J_{CP}=168.7 Hz, CH₂P C: exo), 49.73 (s, CH₂ bridge), 46.75 (s, exo CH norb), 46.44 (s, CH₂ bridge), 45.89 (s, endo CH norb), 43.15 (s, endo CH norb), 42.93 (s, exo CH norb), 42.63 (s, endo CH norb), 41.77 (s, exo CH norb), 30.57 (s, exo CH₂ norb), 29.51 (s, endo CH₂ norb), 16.53 (d, ³J_{CP}=5.9 Hz, OCH₂CH₃); ³¹P NMR (CDCl₃) δ (ppm): 19.47; HR-MS (MeOH, ESI, m/z): calcd. 288.28, found 288.41 [M].

### Step 3: Preparation of 6-(2,3-bis(5-norbornen-2-ylcarbonyloxy)propylthio)hexane-1,1-diyl-bis(phosphonic acid) (bisPhn-6C-bisNorb) (E6)

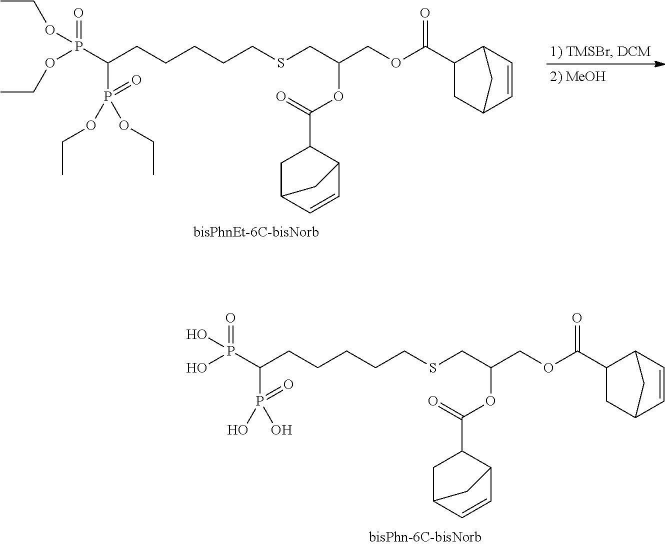 [SMILES: CCOP(=O)(OCC)C(CCCCCSCC(COC(=O)C1CC2C=CC1C2)OC(=O)C1CC2C=CC1C2)P(=O)(OCC)OCC ; O=C(OCC(CSCCCCCC(P(=O)(O)O)P(=O)(O)O)OC(=O)C1CC2C=CC1C2)C1CC2C=CC1C2]

The synthesis was conducted in analogy to Synthesis Example 3, Step 2, from bisPhnEt-6C-bisNorb (1 eq., 10.6 g, 4 mmol) with TMSBr (2.3 eq., 1.2 ml, 9.2 mmol) to isolate 8.8 g (99% of th.) of the desired phosphonic acid bisPhn-6C-bisNorb as a deeply red viscous liquid.

¹H NMR (400 MHZ, CD₃OD) δ (ppm): 6.22-5.84 (m, 4H, 2×CH═CH), 5.22-4.94 (m, 1H, CHO), 4.51-4.23 (m, 1H, CH norb), 4.23-4.01 (m, 3H, CHCH₂O, CH norb), 3.22-3.15 (m, 1H, CH norb), 3.10-2.84 (m, 4H, CH norb, SCH₂CH), 2.75-2.60 (m, 2H, CH₂CH₂S), 2.60-2.49 (m, 2H, 2×CHC═O), 2.33 (tt, ³J_{HH}=24.2, ²J_{HP}=6.1 Hz, 1H, P₂CHCH₂), 2.28-2.13 (m, 2H, PCHCH₂), 1.98-1.85 (m, 4H, 2×CH₂ norb), 1.66-1.44 (m, 4H, 2×CH₂ bridge norb), 1.44-1.34 (m, 4H, CH₂CH₂CH₂S), 1.28-1.20 (m, 2H, PCHCH₂CH₂); ¹³C-NMR (101 MHZ, CD₃OD) δ (ppm): 176.00 (m, C═O), 174.36 (m, C═O), 138.42-138.14 (m, CH═CH), 138.03-137.75 (m, CH═CH), 135.76 (m, CH═CH), 132.64-132.26 (m, CH═CH), 71.24-70.21 (m, CHO), 63.93 (m, CHCH₂O), 49.71 (s, CH₂ bridge), 46.72 (s, CH₂ norb), 46.49 (s, CH₂ norb), 45.82 (m, CHC═O), 43.80-43.02 (m, CH norb), 42.62 (m, CH norb), 41.75 (m, CH norb), 37.94 (t, ¹J_{CP}=128.7 Hz, P₂CH), 32.90-32.13 (m, SCH₂CH), 30.44 (s, CH₂SCH₂), 29.63-28.96 (m, CH₂CH₂S), 28.53 (t, ³J_{CP}=6.6 Hz, PCHCH₂CH₂), 28.30 (m, CH₂CH₂CH₂S), 25.23 (1, ²J_{CP}=4.8 Hz, P₂ CHCH₂); ³¹P NMR (CD:OD) δ (ppm): 22.73; HR-MS (MeOH, ESI, m/z): calcd. 592.58, found 592.58.

### Example 7—Preparation of 3-(3-(3,4-dihydroxyphenyl)propylthio)propane-1,2-diylbis(norbornen-2-carboxylate) (Catechol-3C-bisNorb) (E7)

### Step 1: Preparation of 5-allyl-2,2-dimethyl-1,3-benzodioxol (Catechol^{prot}-3C-Enyl)

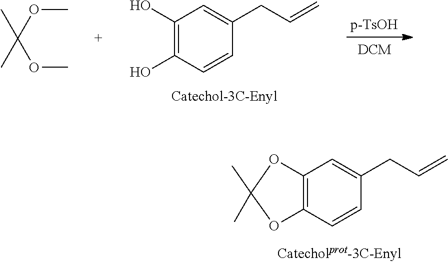

A mixture of Catechol-3C-Enyl (1 eq., 9.8 ml, 57 mmol) from Synthesis Example 11, 2,2-dimethoxypropane (2 eq., 9.9 ml, 114 mmol), and p-toluenesulfonic acid (p-TsOH) (0.06 eq., 0.2 g, 1.14 mmol) in 350 ml of DCM was stirred at room temperature for 20 h, then the solution was washed with a saturated NaHCO₃ solution (3×100 ml) and the aqueous layers with ethyl acetate (3×80 ml). The combined organic layers were dried over MgSO₄ and concentrated to obtain 16.3 g (94% of th.) of the desired compound Catechol^{prot}-3C-Enyl as a brown viscous liquid.

DC (PE) R_{f}=0.27; ¹H NMR (400 MHZ. CDCl₃) δ (ppm): 6.87-6.52 (m, 3H, CH Ar), 6.07-5.80 (m, 1H, CH₂CH═CH₂), 5.15-4.97 (m, 2H, CH₂CH═CH₂), 3.37-3.19 (m, 2H, CH₂CH═CH₂), 1.67 (s, 6H, CH₃); ¹³C-NMR (101 MHz, CDCl₃) δ (ppm): 147.62 (s, CO), 145.77 (s, CO), 137.91 (s, CH═CH₂), 133.35 (s, C_{q}CH₂CH), 120.77 (s, CH Ar), 115.7 (s, CH═CH₂), 115.66 (s, C_{q}(CH₃)₂), 109.02 (s, CH Ar), 108.11 (s, CH Ar), 40.11 (s, C_{q}CH₂CH), 25.97 (s, C_{q}(CH₃)₂).

### Step 2: Preparation of 3-(3-(2,2-dimethyl-1,3-benzodioxol-5-yl)propylthio)propane-1,2-diol (Catechol^{prot}-3C-thioglyc)

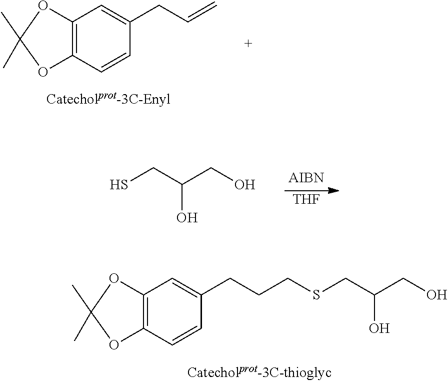 [SMILES: C=CCc1ccc2c(c1)OC(C)(C)O2 ; OCC(O)CS ; CC1(C)Oc2ccc(CCCSCC(O)CO)cc2O1 ; "THF"]

Catechol^{prot}-3C-Enyl (1 eq., 15 g, 79 mmol) and thioglycerol (1.5 eq., 12.8 g, 118.5 mmol) were dissolved in 250 ml of dry THF under argon. The mixture was degassed with argon form 45 min, then 0.2 g AIBN (0.02 eq., 0.92 mmol) were added and the reaction mixtures was stirred at 75° C. for 24 h while the reaction progress was monitored by DC and ³¹P NMR. Subsequently, the solution was concentrated under reduced pressure, and the obtained raw product was purified by column chromatography (EE:PE, 50:50) to obtain 7.26 g (31% of th.) of the desired protected product Catechol^{prot}-3C-thioglyc as a yellow viscous liquid.

DC (EE:PE 80:20) R_{f}=0.54; ¹H NMR (400 MHZ, CDCl₃) δ (ppm): 6.71-6.48 (m, 3H, Ar—H), 3.87-3.63 (m, 2H, CH₂OH), 3.63-3.42 (m, 1H, CHOH), 2.70-2.40 (m, 6H, C_{q}CH₂CH₂CH₂SCH₂), 1.97-1.73 (m, 2H, C_{q}CH₂CH₂), 1.65 (s, 6H, C_{q}(CH₃)₂); ¹³C-NMR (101 MHZ, CDCl₃) δ (ppm): 147.58 (s, CO), 145.74 (s, CO), 134.46 (s, C_{q}CH₂CH₂), 120.67 (s, CH Ar), 117.72 (s, C_{q}(CH₃)₂), 108.74 (s, CH Ar), 108.07 (s, CH Ar), 70.02 (s, CHOH), 65.51 (s, CH₂OH), 35.83 (SCH₂CH), 34.55 (s, C_{q,Ar}CH₂), 31.66 (s, C_{q,Ar}CH₂CH₂), 31.48 (s, CH₂SCH₂CH, 25.95 (s, C_{q}(CH₃)₂).

### Step 3: Preparation of 3-(3-(2,2-dimethyl-1,3-benzodioxol-5-yl)propylthio)propane-1,2-diyl-bis(norbornen-2-carboxylate) (Catechol^{prot}-3C-bisNorb)

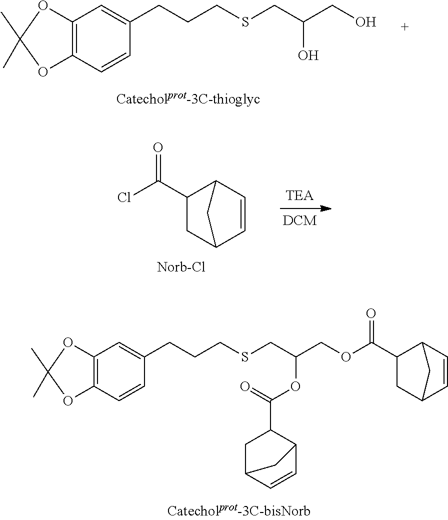 [SMILES: CC1(C)Oc2ccc(CCCSCC(O)CO)cc2O1 ; O=C(Cl)C1CC2C=CC1C2 ; CC1(C)Oc2ccc(CCCSCC(COC(=O)C3CC4C=CC3C4)OC(=O)C3CC4C=CC3C4)cc2O1]

The synthesis was conducted in analogy to Example 3, Step 2, from Catechol^{prot}-3C-thioglyc (1 eq., 6.0 g, 20 mmol) with Norb-Cl (3 eq., 12.3 g, 60 mmol) and TEA (3.2 eq., 8.9 ml, 64 mmol) to isolate, by column chromatography (PE:EE 95:5), 4.5 g (42% of th.) of the desired protected product Catechol^{prot}-3C-bisNorb as a brown viscous liquid.

DC (EE:PE, 1:9) R_{f}=0.54; ¹H NMR (400 MHZ, CDCl₃) δ (ppm): 6.68-6.48 (m, 3H, 3×Ar—H), 6.23-5.86 (m, 4H, 2×CH═CH), 5.24-5.00 (m, 1H, CHO), 4.50-4.19 (m, 2H, CH₂O), 3.28-2.86 (m, 4H, (m, 4×CH norb), 2.77-2.47 (m, 6H, C_{q}CH₂CH₂CH₂SCH₂), 2.22 (m, 2H, 2×CHCO norb) 1.99-1.77 (m, 3H, CH₂ norb, C_{q}CH₂CH₂), 1.65 (s, 6H, C_{q}(CH₃)₂), 1.58-1.32 (m, 5H, CH₂ norb, 2×CH₂ bridge); ¹³C-NMR (101 MHZ, CDCl₃) δ (ppm): 175.81 (s, C═O), 175.48 (s, C═O), 147.54 (s, C—OC_{q}), 145.69 (s, C—OC_{q}), 138.21 (s, CH═CH), 135.74 (s, CH═CH), 134.52 (s, C_{q}CH₂CH₂), 120.63 (s, CH Ar), 117.59 (s, C_{q}(CH₃)₂), 108.72 (s, CH Ar), 108.01 (s, CH Ar), 70.59 (s, CHO), 63.92 (s, CH₂O), 49.69 (s, CH₂ bridge), 46.45 (s, CH norb), 45.85 (s, CH norb), 43.51-43.10 (m, CHC═O norb), 42.82-42.42 (m, CH norb), 41.73 (s, CH norb), 34.48 (s, SCH₂CH), 32.38 (s, Co_{q, Ar}CH₂), 32.01 (s, CH₂SCH₂CH), 30.43 (s, CH₂ norb), 29.31 (s, C_{q,Ar}CH₂CH₂), 25.91 (s, C_{q}(CH₃)₂).

### Step 4: Preparation of 3-(3-(3,4-dihydroxyphenyl)propylthio)propane-1,2-diylbis(norbornen-2-carboxylate) (Catechol-3C-bisNorb) (E7)

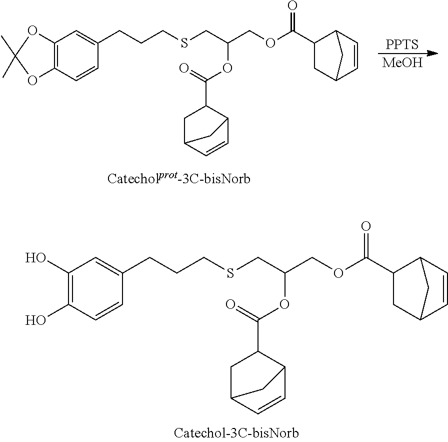

A mixture of Catechol^{prot}-3C-bisNorb (1 eq., 2.9 g, 5.4 mmol) and pyridinium-p-toluene-sulfonate PPTS (0.1 eq., 0.14 g, 0.54 mmol) in 30 ml of dry methanol was stirred under reflux overnight. The solvent was evaporated, and the raw product was dissolved in 50 ml of DCM. The organic layer was washed with NaHCO₃ (3×75 ml) and dried over MgSO₄ to isolate 1.73 g (64% of th.) of the desired product Catechol-3C-bisNorb as a brown viscous liquid.

DC (EE:PE, 1:1) R_{f}=0.67; ¹H NMR (400 MHZ, CDCl₃) δ (ppm): 6.91-6.51 (m, 3H, 3×Ar—H), 6.27-5.87 (m, 4H, 2×CH═CH), 5.25-5.06 (m, 1H, CHO), 4.59-4.33 (m, 1H, CH₂O), 4.30-4.06 (m, 1H, CH₂O), 3.27-2.86 (m, 4H, 4×CH norb), 2.83-2.41 (m, 6H, C_{q}CH₂CH₂CH₂SCH₂), 2.40-2.16 (m, 2H, 2×CHCO norb), 2.03-1.73 (m, 3H, CH₂ norb, C_{q}CH₂CH₂), 1.57-1.33 (m, 5H, CH₂ norb, 2×CH₂ bridge); ¹³C-NMR (101 MHz, CDCl₃) δ (ppm): 176.47 (s, C═O), 175.09 (s, C═O), 143.63 (s, C—OC_{q}), 142.35 (s, C—OC_{q}), 138.28 (s, CH═CH), 135.74 (s, CH═CH), 133.77 (s, C_{q}CH₂CH₂), 120.89 (s, CH Ar), 115.61 (s, CH Ar), 115.35 (s, CH Ar), 70.85 (s, CHO), 64.26 (s, CH₂O), 49.76 (s, CH₂ bridge), 46.68 (s, CH norb), 46.51 (s, CH₂ bridge), 45.96 (s, CH norb), 43.53 (s, CHC═O norb), 43.44 (s, CHC═O norb), 43.27 (s, CH norb), 42.68 (s, CH norb), 41.75, 33.44 (SCH₂CH), 32.58 (s, C_{q,Ar}CH₂), 31.73 (s, CH₂SCH₂CH), 30.81 (s, 2×CH₂ norb), 29.38 (s, C_{q,Ar}CH₂CH₂); elementary analysis: calcd. for C₂₈H₃₄O₆S: C-67.44, H-6.87, O-19.25, S6.43, found: C-67.28, H-6.32, O-19.28, S-5.96.

### Example 8—Preparation of ethylenediaminetetraacetic acid-bis(2-(3,4-dihydroxyphenyl)ethyl)amide-bis(2-(5-norbornen-2-yl)ethyl)amide (bisCatechol-EDTA-bisNorb) (E8)

### Step 1: Preparation of ethylenediaminetetraacetic acid dianhydride (EDTA dianhydride)

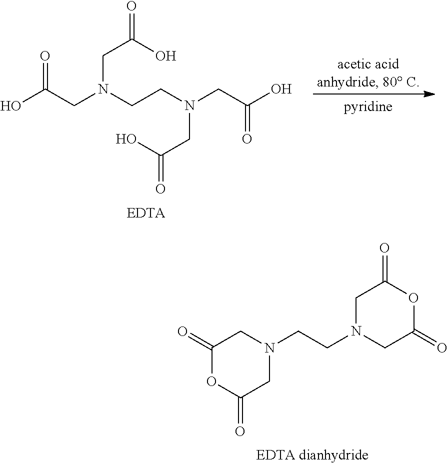 [SMILES: O=C(O)CN(CCN(CC(=O)O)CC(=O)O)CC(=O)O ; O=C1CN(CCN2CC(=O)OC(=O)C2)CC(=O)O1]

The synthesis known from literature was carried out as described in Peng et al., Org. Biomol. Chem. 15(30), 6441-6446 (2017) by reacting ethylenediaminetetraacetic acid (EDTA) with acetic acid anhydride in pyridine at 80° C.

DC (PE:EE, 2:1) R_{f}=0.43; Fp.: 192-193ªC; 1H NMR (400 MHZ, DMSO-d) δ (ppm): 3.71 (s, 8H, 4×(CH₂)₂N), 2.66 (s, 4H, NCH₂CH₂N); ¹³C-NMR (101 MHZ, DMSO-d) δ (ppm): 172.20 (s, 4×C═O), 54.71 (s, 4×(CH₂)₂N), 51.37 (s, NCH₂CH₂N).

### Step 2: Preparation of ethylenediaminetetraacetic acid-bis(2-(5-norbornen-2-yl)ethyl)amide (EDTA-bisNorb)

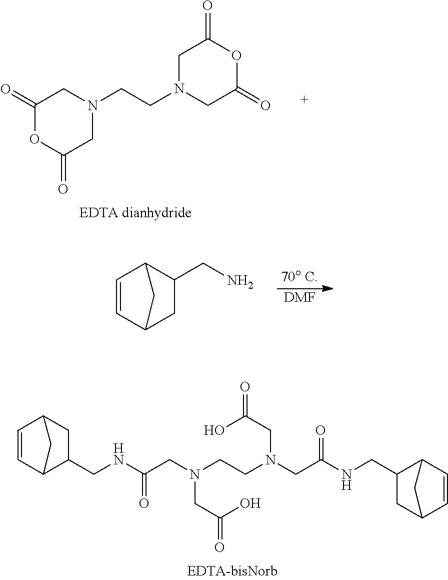

EDTA-dianhydride (1 eq., 4.6 g, 18 mmol) was dissolved in 46 ml of DMF and stirred for 10 min, then 5-norbornen-2-ylmethylamine (2.04 eq., 4.5 g, 36.7 mmol) was slowly added and the reaction mixture was stirred at 70° C. for 24 h. The brown mixture was concentrated, filtered, and treated with 13 ml of chloroform. Addition of 250 ml of diethylether (Et₂O) led to the precipitation of a white solid, followed by removal of the supernatant. The precipitate was triturated twice with 150 ml of Et₂O, removed by filtration and dried in vacuum to obtain 8.9 g (98% of th.) of the desired EDTA-diamide EDTA-bisNorb as a white solid.

Fp.: 148.1-153.2° ° C. (dec.); ¹H NMR (400 MHZ, DMSO-d) δ (ppm): 8.06-7.91 (m, 2H, 2×NH), 6.21-5.91 (m, 4H, 2×CH═CH), 3.35 (s, 4H, 2×NCH₂COOH), 3.20 (s, 4H, 2×NHCOCH₂), 2.88-2.81 (m, 2H, 2×CH₂NH), 2.79-2.73 (m, 6H, 2×CH₂NH, 4×CH norb), 2.71 (s, 4H, NCH₂CH₂N), 2.25-2.15 (m, 2H, 2×CHCH₂NH norb), 1.81-1.70 (m, 2H, 2×CH₂ norb), 1.36-1.27 (m, 2H, 2×CH₂ norb), 1.25-1.02 (m, 4H, 2×CH₂ bridge); ¹³C-NMR (101 MHZ, DMSO-d) δ (ppm): 172.76 (s, 2×COOH), 170.07 (s, 2×NHC═O), 136.94 (s, 2×CH═CH), 132.57 (s, 2×CH═CH), 64.97 (s, 2×NCH₂COOH), 57.81 (s, 2×NCH₂C═O), 55.78 (s, NCH₂CH₂N), 52.50 (s, 2×CH₂ bridge), 49.00 (s, 2×NHCH₂CH), 43.73 (s, 2×CH norb), 41.91 (s, 2×CH norb), 38.47 (s, 2×CH norb), 29.76 (s, 2×CH₂ norb); elementary analysis: calcd. for C₂₆H₃₈N₄O₆. C-62.13, H-7.62, N-11.15. O-19.10; found: C-62.23, H-7.78, N-10.57, O-19.01.

### Step 3: Preparation of ethylenediaminetetraacetic acid-bis(2-(3,4-dihydroxyphenyl)ethyl)amide-bis(2-(5-norbornen-2-yl)ethyl)amide (bisCatechol-EDTA-bisNorb) (E8)

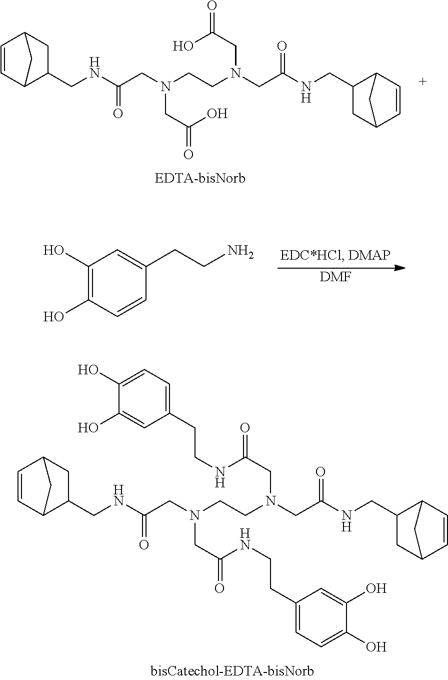

EDTA-bisNorb (1 eq., 7.5 g, 15 mmol), dopamine hydrochloride (5 eq., 11.5 g, 75 mmol) and DMAP (0.4 eq., 0.2 g, 6 mmol) were dissolved in 20 ml of DMF and stirred at −10° ° C. under argon, then a suspension of EDC·HCl in 70 ml of DMF was added dropwise while keeping the temperature under 0° C. After complete addition, the mixture was stirred at room temperature for 48 h, then the solvent was evaporated, the product was precipitated in deionized water, and the precipitate was dried under vacuum to obtain 8.3 g (72% of th.) of the desired EDTA-tetraamide bisCatechol-EDTA-bisNorb as a light brown solid.

Fp.: 112.3-116.5° C.; 1H NMR (400 MHZ, MeOD-d) δ (ppm): 7.90 (s, 2H, 2×NH norb), 6.79-6.75 (s, 2H, 2×NH dopa), 6.71-6.47 (m, 4H, 4×CH Ar), 6.48-6.34 (m, 2H, 2×CH Ar), 6.19-5.79 (m, 4H, 2×CH═CH), 3.52-3.44 (m, 2H, 2×CH₂NH), 3.40 (t, ³J_{HH}=7.2 Hz, 4H, 2×NHCH₂CH₂ dopa), 3.35 (s, 8H, 4×NHCOCH₂N), 3.26-3.06 (m, 2H, 2×CH₂NH), 3.03-2.89 (m, 2H, CH₂NH), 2.88 (s, 4H, NCH₂CH₂N), 2.84-2.76 (m, 4H, 4×CH norb), 2.67 (1, ³J_{HH}=7.2 Hz, 4H, 2×NHCH₂CH₂ dopa), 2.30-2.11 (m, 2H, 2×CHCH₂NH norb), 1.91-1.81 (m, 2H, 2×CH₂ norb), 1.49-1.27 (m, 2H, 2×CH₂ norb), 1.25-1.07 (m, 4H, 2×CH₂ bridge); ¹³C-NMR (101 MHZ, MeOD-d) δ (ppm): 146.28 (s, 2×C_{q}OH), 144.85 (s, 2×C—OH), 138.54 (s, 2×CH═CH), 133.32 (s, 2×CH═CH), 131.84 (s, 2×C_{q}CH₂CH₂NH), 121.06 (s, CH Ar), 116.98 (s, CH Ar), 116.47 (s, CH Ar), 58.79 (s, NCH₂C═O), 57.54 (s, NCH₂C═O), 56.86 (s, NCH₂C═O), 56.39 (s, NCH₂C═O), 54.08 (s, NCH₂CH₂N), 49.66 (s, 2×CH₂ bridge), 45.46 (s, CH norb), 44.75-44.32 (m, 2×NHCH₂CH), 43.70 (s, CH norb), 42.37-41.43 (m, C_{q}CH₂CH₂NH), 40.06-39.70 (m, CH norb), 36.03-35.45 (m, C_{q}CH₂CH₂NH), 31.55-30.87 (m, 2×CH₂ norb); elementary analysis: calcd. for C₄₂H₅₆N₆O₈: C-65.26. H-7.30, N-10.87, O-16.56; found: C-65.80, H-7.33, N-10.85, O-16.29.

### Example 9—Preparation of diethylenetriaminepentaacetic acid-bis(2-(5-norbornen-2-yl)ethyl)amide-tris(2-(3,4-dihydroxyphenyl)ethyl)amide (triCatechol-DTPA-bisNorb) (E9)

### Step 1: Preparation of ethylenediaminetetraacetic acid dianhydride (DTPA-dianhydride)

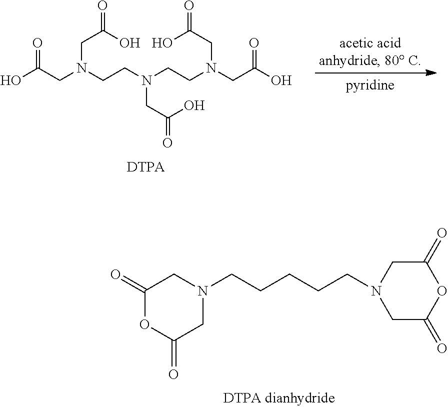 [SMILES: O=C(O)CN(CCN(CC(=O)O)CC(=O)O)CCN(CC(=O)O)CC(=O)O ; O=C1CN(CCCCCN2CC(=O)OC(=O)C2)CC(=O)O1]

The synthesis was conducted in analogy to Example 8, Step 1, according to the instructions in Peng et al., Org. Biomol. Chem. 15(30), 6441-6446 (2017) by reacting diethylenetriaminepentaacetic acid (DTPA) with acetic acid anhydride.

Fp.: 182.1-184.2° C.; 1H NMR (400 MHZ, DMSO-d) δ (ppm): 3.71 (s, 8H, 4×(CH₂)₂N), 3.29 (s, 2H, NCH₂COOH), 2.75 (t, ³J_{HH}=6.2 Hz, 4H, NCH₂CH₂N), 2.59 (t, ³J_{HH}=5.8 Hz, 4H, NCH₂CH₂N); ¹³C-NMR (101 MHZ, DMSO-d) δ (ppm): 171.99 (s, CH₂COOH), 165.81 (s, 4×C═O), 54.56 (s, CH₂COOH), 52.61 (s, 4×NCH₂COO), 51.79 (s, 2×NCH₂CH₂N), 50.74 (s, 2×NCH₂CH₂N).

### Step 2: Preparation of diethylenetriaminepentaacetic acid-bis(2-(5-norbornen-2-yl)ethyl)amide (DTPA-bisNorb)

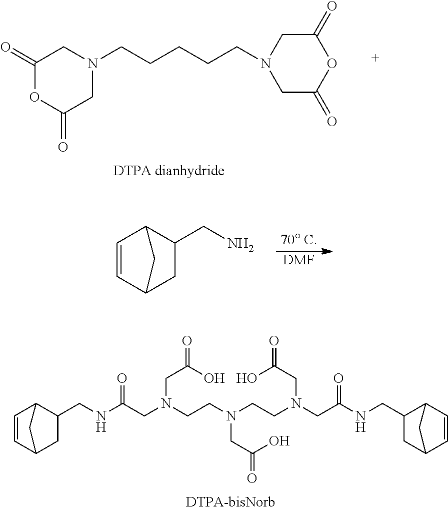

The synthesis was conducted in analogy to Example 8, Step 2, from DTPA dianhydride (1 eq., 23.4 g, 18 mmol) and 5-norbornen-2-ylmethylamine (2.04 eq., 4.5 g, 36.7 mmol) in 70 ml of DMF. The raw product was washed with 150 ml of DCM and 200 ml of Et₂O and dried und vacuum to isolate 9.1 g (84% of th.) of the desired diamide DTPA-bisNorb as a brown solid.

Fp.: 83.5-87.6° C.; ¹H NMR (400 MHZ, DMSO-d) δ (ppm): 8.05 (t, ³J_{HH}=5.8 Hz, 2H, 2×NH), 6.18-5.93 (m, 4H, 2×CH═CH), 3.44 (s, 2H, NCH₂COOH), 3.37 (s, 4H, 2×CHCH₂NH), 3.23 (s, 4H, 2×COCH₂N), 3.20-3.04 (m, 4H, 2×NHCOCH₂), 2.99-2.94 (m, 4H, 2×NCH₂CH₂N), 2.89-2.81 (m, 4H, 2×NCH₂CH₂N), 2.78 (s, 2H, 2×CH norb), 2.75-2.67 (m, 2H, 2×CH norb), 2.26-2.17 (m, 2H, 2×CHCH₂NH norb), 1.80-1.73 (m, 2H, 2×CH₂ norb), 1.34-1.28 (m, 2H, 2×CH₂ norb), 1.24-1.12 (m, 4H, 2×CH₂ bridge); 1ºC-NMR (101 MHZ, DMSO-d) δ (ppm): 172.75 (s, 2×CH₂COOH), 171.99 (s, CH₂COOH), 169.82 (s, 2×NHCOCH₂N), 136.83 (s, 2×CH═CH), 132.62 (s, 2×CH═CH), 57.58 (s, 2×NCH₂ COOH), 55.30 (s, NHCOCH₂N), 54.92 (s, NCH₂COOH), 52.27 (s, 2×NCH₂CH₂N), 51.09 (s, 2×NCH₂CH₂N), 48.95 (s, 2×CH₂ bridge), 43.69 (s, 2×CH norb), 42.44 (s, 2×NHCH₂CH), 41.88 (s, 2×CH norb), 38.39 (s, 2×CH norb), 29.74 (s, 2×CH₂ norb); elementary analysis: calcd. for C₃₀H₄₅N₅O₈L C-59.68, H-7.51, N-11.60, O-21.20; found: C-59.58, H-7.46, N-11.69, O-21.35.

### Step 3: Preparation of diethylenetriaminepentaacetic acid-bis(2-(5-norbornen-2-yl)ethyl)amide-tris(2-(3,4-dihydroxyphenyl)ethyl)amide (triCatechol-DTPA-bisNorb) (E9)

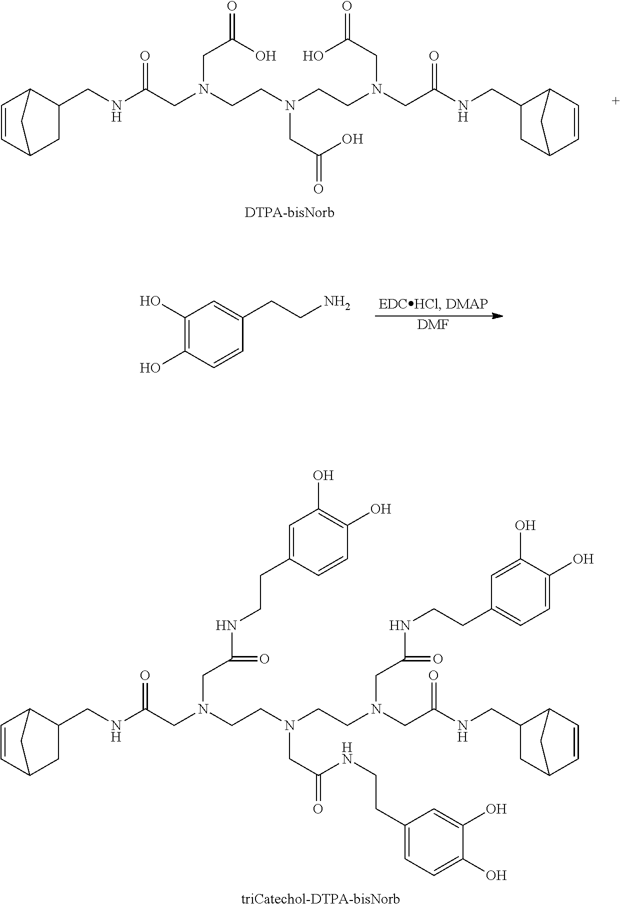

The synthesis was conducted in analogy to Example 8, Step 3, from DTPA-bisNorb (1 eq., 6.9 g, 11.5 mmol), dopamine hydrochloride (4 eq., 8.7 g, 46 mmol), and DMAP (0.6 eq., 0.8 g, 6.9 mmol) in 80 ml of DMF with a suspension of EDC·HCl (3.5 eq., 7.7 g, 40.25 mmol) in 220 ml of DMF. The raw product was dissolved in ethanol and precipitated in deionized water to obtain 2.9 g (25% of th.) of a brown, highly viscous liquid. 1H NMR (400 MHZ, MeOD-d) δ (ppm): 7.88 (s, 2H, 2×NH norb), 6.89-6.26 (m, 9H, 9×CH Ar), 6.16-5.74 (m, 4H, 2×CH═CH), 3.59 (m, 2H, 2×CH₂NH), 3.30 (t, ³J_{HH}=7.2 Hz, 6H, 3×NHCH₂CH₂ dopa), 3.16-2.93 (m, 12H, 5×NHCOCH₂N), 3.04-2.95 (m, 2H, 2×CH₂NH), 2.72-2.66 (m, 8H, 2×NCH₂CH₂N), 2.57 (t, ³J_{HH}=7.2 Hz, 6H, 3×NHCH₂CH₂ dopa), 2.25-2.12 (m, 2H, 2×CHCH₂NH norb), 1.87-1.67 (m, 2H, 2×CH₂ norb), 1.38-1.25 (m, 2H, 2×CH₂ norb), 1.24-1.01 (m, 4H, 2×CH₂ bridge); ¹³C-NMR (101 MHZ, MeOD-d) δ (ppm): 173.22 (s, 3×NHCOCH₂N dopa), 172.90 (s, 2×NHCOCH₂N norb), 146.27 (s, 3×C_{q}OH), 144.82 (s, 3×C_{q}OH), 138.50 (s, 2×CH═CH), 133.35 (s, 2×CH═CH), 131.86 (s, 2×C_{q}CH₂CH₂NH), 121.12 (s, 3×CH Ar), 117.00 (s, 3×CH Ar), 116.62 (s, 3×CH Ar), 59.78 (s, 3×NCH₂C═O), 58.33 (s, NCH₂CH₂N), 56.46 (s, NCH₂CH₂N), 50.37 (s, 2×CH₂ bridge), 45.49 (s, CH norb). 44.41 (m, 3×NHCH₂CH), 43.69 (s, CH norb), 42.41-41.73 (m, C_{q}CH₂CH₂NH), 39.97 (m, CH norb), 37.30 (m, CH norb), 36.01-35.28 (m, 3×C_{q}CH₂CH₂NH), 31.14 (m, 2×CH₂ norb); elementary analysis: calcd. for C₅₄H₇₂N₈O₁₁: C-64.27, H-7.19, N-11.10, O-17.44; found: C-64.27, H-7.86, N-11.64, O-18.27.

### Examples 10 to 36, Comparative Examples 1 to 30—Preparation and Testing of Polymerizable Compositions

### Preparation of the Surfaces to be Coated

Three different substrates were prepared to measure the shear bond strength (SBS) of polymerizable compositions.

### 1) Hydroxyapatite

This mineral with the chemical formula Ca₅[OH|(PO₄)₃] represents the main component of the inorganic hard substance in bones and is therefore usually used as a model substance for test series with bone adhesives. For the purpose herein, hydroxyapatite (HAP) powder was obtained from Lithoz GmbH (Vienna, Austria) and cold-isostatically pressed into pellets with 400 kN for 1 min. The pellets thus obtained were then sintered in a laboratory oven from Carbolite Gero GmbH (Neuhausen, Germany) according to the following temperature program: with 2 K/min to 500° C. 30 min waiting time, with 2 K/min to 1250° C., 2 h waiting time, then cooling to room temperature with 3 K/min.

### 2) Titanium Dioxide

Titanium dioxide (TiO₂) powder (Ø 200 nm) from Cinkarna (Celje, Slovenia) was lasers-intered to TiO₂ pellets using a Hammer Lab35 Metal 3D Printer from Incus GmbH (Vienna, Austria).

### 3) Bovine Bones

For conducting ex-vivo measurements of bone material, parts of bovine hooves surrounding the distal end of the second phalanx, were cut into discs with a thickness of 1 cm with a band. The bovine toe bone discs were then cut into smaller pieces and placed in PBS buffer and frozen at −80° C. for storage until use.

All three substrates were embedded in epoxy resin before coating with the polymerizable compositions, and their surfaces were polished smoothly with SiC wet sandpaper with a grain size of 400.

### Preparation of the Polymerizable Compositions

By simply mixing one of the adhesion-providing primers prepared in the above Synthesis Examples and Examples with multifunctional monomers, multifunctional thiols and a standard radical initiator known from literature to be suitable for use in bone adhesives, one- and two-component compositions were prepared.

### Examples 10 to 24, Comparative Examples 1 to 18—Two-Component Compositions

To examine the tolerability of the new primers with regard to the other components of such compositions, several two-component compositions were initially prepared based on trimethylolpropane-tris(3-mercaptopropionate) (TMPMP) as the thiol and bis-(4-methoxybenzoyl)diethylgermanium, a radical photoinitiator commercially available under the trademark Ivocerin® from Ivoclar Vivadent (Schaan, Liechtenstein).

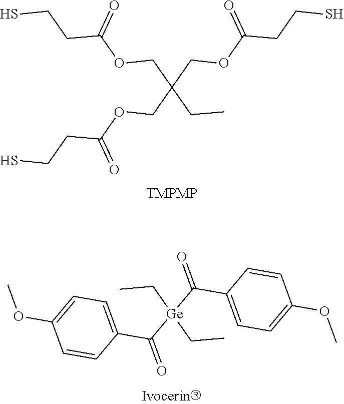

Monomers examined where the trifunctional, relatively rigid triallylisocyanurate 1,3,3-triallyl-1,3,5-triazine-2,4,6-trione (TATATO) and the difunctional, biologically degradable vinyl ester O,O′-(hexahydrofuro[3,2-b]furan-3,6-diyl)divinyladipate (glucitoldivinyl-adipate, GDVA).

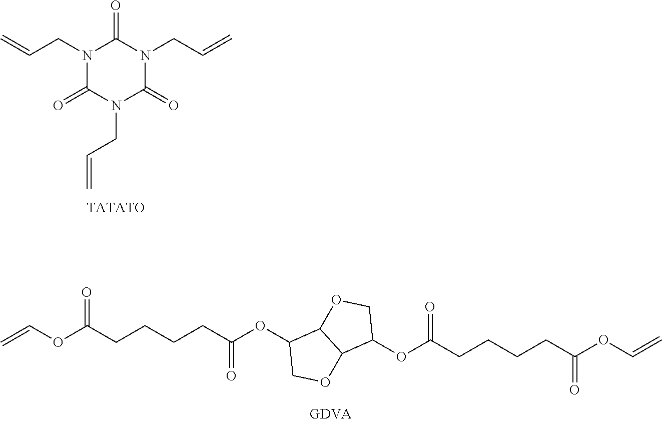 [SMILES: C=CCn1c(=O)n(CC=C)c(=O)n(CC=C)c1=O ; C=COC(=O)CCCCC(=O)OC1COC2C(OC(=O)CCCCC(=O)OC=C)COC12]

Mixtures of monomer, thiol, initiator, and primer were prepared as the first component and those without primer as the second component, herein referred to as “primer formulation”, PF, or “matrix formulation”, MF. The primer used was initially the common primer from Synthesis Example 1, 2,2-bis(allyloxymethyl)propanamido)methyl)phosphonic acid (BAPA^{Phn}) (S1).

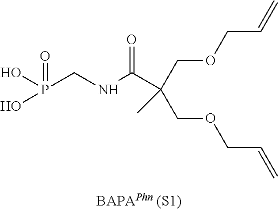 [SMILES: C=CCOCC(C)(COCC=C)C(=O)NCP(=O)(O)O]

The following Table 1 shows the amounts of the individual compounds in wt % based on the total mixture, wherein each formulation additionally contained 0.02 wt % of pyrogallol (1,2,3-trihydroxybenzene) as a thiol-ene stabilizer.

TABLE 1
First (PF) and second (MF) components of two-component compositions
according to the state of the art with varying monomers

| | BAPA^{Phn} | Monomer | TMPMP | Ivocerin |
|---|---|---|---|---|
| Formulation | [wt %] | [wt %] | [wt %] | [wt %] |
| PF1 | 15 | 12.5 TATATO | 71 | 1.5 |
| PF2 | 15 | 12.5 GDVA | 71 | 1.5 |
| MF1 | — | 32 TATATO | 67 | 1 |
| MF2 | — | 32 GDVA | 67 | 1 |

From these four formulations, three combinations were mixed as two-component compositions and used for coating substrates, which coatings were then cured and examined for their shear bond strength:
Comparative Example 1 (C1): PF1+MF1
Comparative Example 2 (C2): PF2+MF1
Comparative Example 3 (C3): PF1+MF2

For this purpose, hydroxyapatite or titanium dioxide discs prepared and polished as described above were first coated with the respective primer formulation PF by applying the mixture to the surface and scrubbing it for 20 s with a micro brush, letting it air-dry for 10 s, and then irradiating it for 10 s with a LED polymerization lamp Bluephase C8 by Ivoclar Vivadent (Schaan, Liechtenstein) with an emission in a wavelength range of 385-515 nm and a power of 720 mW cm-2. The respective matrix formulation MF was applied as the second component to the thus-cured first component (with the aid of a mold delimiting the edges of the surface in order to prevent the formulation from flow off), and cured by irradiation with the LED lamp for 20 s.

Then, the samples were placed in modified simulated body fluid (m-SBF) and stored for 24 h at 37° C., followed by an examination of the shear bond strength (SBS) of the cured composition using a VTSYIQI SF-30 digital force gauge with a V-shaped test rod.

As can be clearly seen from the results graphically show in FIG. 1A, from the three samples, only the combination of PF1 and MF1 of Comparative Example 1, where both components contained TATATO as monomer, showed good results in the shear bond strength test. Those of Comparative Examples 2 and 3, where only one of the two formulations used GDVA as the monomer, however, did not achieve even remotely satisfactory strength. Therefore, no tests with a combination of both GDVA-containing formulations, PF2+MF2, were performed.

Subsequently, the inventors performed a first polymerizable composition, wherein in analogy to the standard primer BAPA^{Phn} examined before, one of the new primers with a similar spacer length and also two polymerizable groups PG and an adhesion motif AM was initially tested, namely 6-(2,3-bis(5-norbornen-2-ylcarbonyloxy)propylthio)hexylphosphonic acid (Phn-6C-bisNorb) (E5) from Example 5 with a phosphonic acid and two norbornenyl moieties:

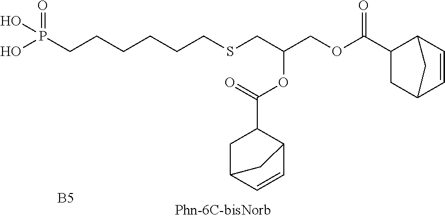 [SMILES: O=C(OCC(CSCCCCCCP(=O)(O)O)OC(=O)C1CC2C=CC1C2)C1CC2C=CC1C2 ; "B5"]

The preparation of formulations with the two monomers of TATATO and GDVA and Ivocerin as the initiator and their combination to obtain two-component compositions was also conducted in analogy to the above Comparative Examples 1 to 3, as specified below.

TABLE 2
First (PF) and second (MF) components of two-component compositions
with varying monomers

| | Phn-6C-bisNorb (E5) | Monomer | TMPMP | Ivocerin |
|---|---|---|---|---|
| Formulation | [wt %] | [wt %] | [wt %] | [wt %] |
| PF1 | 15 | 12.5 TATATO | 71 | 1.5 |
| PF2 | 15 | 12.5 GDVA | 71 | 1.5 |
| MF1 | — | 32 TATATO | 67 | 1 |
| MF2 | — | 32 GDVA | 67 | 1 |

These were again used to mix three combinations as two-component compositions:
Example 10 (E10): PF1+MF1
Example 11 (E11): PF2+MF1
Example 12 (E12): PF1+MF2

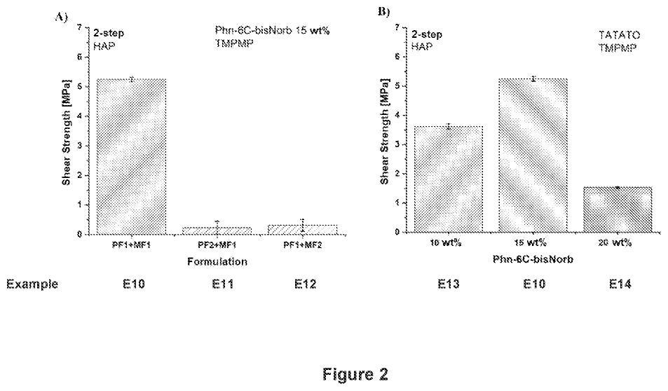

These compositions were subsequently applied to the Example polished hydroxyapatite discs in the same way as described above, (pre)cured, and the shear bond strength of the coating was tested. The results are shown in FIG. 2A.

A comparison of the results of Comparative Examples 1 to 3 in FIG. 1A and of Examples 10 to 12 in FIG. 2A clearly shows that both GDVA-containing compositions of Examples 11 and 12 showed only insignificantly better values for the shear bond strength compared to those of Comparative Examples 2 and 3, however, the two-component composition of Example 1 without GDVA and with Phn-6C-bisNorb as the primer resulted in more than three times the shear bond strength compared to BAPA^{Phn} in Comparative Example 1, namely 5.25±0.08 MPa according to the present invention compared to only 1.64±0.15 MPa according to the state of the art.

### Variation of the Primer Concentration

Subsequently, the common primer BAPA^{Phn} as well as Phn-6C-bisNorb (E5) were used in experiments with varying primer concentrations in the formulations. Here, in the above primer formulations PF1, each containing 15 wt % of primer, the amounts thereof were each reduced or increased by 5 wt % and the amounts of monomer (TATATO) and thiol (TMPMP) were also adapted correspondingly. These primer formulations, referred to, depending on the amount of primer, as PF10, PF15 and PF20, were each combined with the TATAO-containing matrix formulation MF1 to obtain two-component compositions. The exact compositions of the individual formulations are given in the overleaf Table 3.

TABLE 3
First (PF) and second (MF) components of two-component compositions according
to the state of the art and in inventive uses with varying primer amounts

| | | BAPA^{Phn} | Phn-6C-bisNorb (E5) | TATATO | TMPMP | Ivocerin |
|---|---|---|---|---|---|---|
| Example | Formulation | [wt %] | [wt %] | [wt %] | [wt %] | [wt %] |
| Comparative Example 1 (C1) | PF₁₅ | 15 | — | 12.5 | 71.0 | 1.5 |
| Comparative Example 4 (C4) | PF₁₀ | 10 | — | 15.0 | 73.5 | 1.5 |
| Comparative Example 5 (C5) | PF₂₀ | 20 | — | 10.0 | 68.5 | 1.5 |
| Example 10 (E10) | PF₁₅ | — | 15 | 12.5 | 71.0 | 1.5 |
| Example 13 (E13) | PF₁₀ | — | 10 | 15.0 | 73.5 | 1.5 |
| Example 14 (E14) | PF₂₀ | — | 20 | 10.0 | 68.5 | 1.5 |
| All foregoing examples | MF1 | — | — | 32.0 | 67.0 | 1.0 |

The four new two-component compositions of Comparative Examples 4 and 5 as well as of Examples 13 and 14 were applied to hydroxyapatite surfaces in the same way as above, (pre)cured, and examined with regard to the shear bond strength of the coating. The results thus obtained are, together with those previously obtained for the compositions of Comparative Example 1 and Example 10 each containing 15 wt % of primer, shown in FIGS. 1B and 2B.

A comparison of these two diagrams again shows a similar trend for the comparative examples as well as the examples according to the present invention: In both cases, the new compositions resulted, with less (10 wt %) or more (20 wt %) of primer in the primer formulations, in a lower shear strength than before with 15 wt % of the respective primers each, wherein in each case, 10 wt % resulted in better values than 20 wt % of primer. Similar to the inventive Example 10 above with 15 wt % of Phn-6C-bisNorb in the primer formulation, Example 13 of the invention with 10 wt % of primer also shows a more than three times higher shear bond strength than the corresponding composition according to the state of the art with 10 or 15 wt % of BAPA^{Phn} (3.62±0.09 MPa vs. 0.98±0.26 MPa). And for Example 14, the value is also more than twice as high as that of Comparative Example 5, each containing 20 wt % of primer (1.64±0.15 MPa vs. 0.75±0.21 MPa).

In summary, it can on the one hand be stated that a relatively rigid, trifunctional monomer like TATATO, which consequently has a stronger cross-linking effect, is clearly to be preferred over a difunctional and clearly more flexible monomer like GDVA. On the other hand, that approximately 15 wt % of primer in a primer formulation seem to be most suitable as the first component in a two-component composition to achieve high shear bond strengths of the cured compositions.

However, a content of 20 wt % and, in particular, only 10 wt % of primer also resulted in good or at least acceptable shear bond strengths, so that in general a content of 5 to 25 wt % of primer in the primer formulation of two-component compositions seem to be preferred, the content more preferably being approximately 7 wt % to approximately 17 wt %, in particular approximately 12 to 15 wt %.

### Variation of the Thiol

Again using 15 wt % of primer in the primer formulation of a two-component composition, the thiol contained in the two components was then varied.

For this purpose, in both of the formulations PF and MF of Comparative Example 1 and Example 10 to be combined, the thiol TMPMP was replaced by the same amounts of either tris[2-(3-mercaptopropionyloxy)ethyl]isocyanurate (TEMPIC) or dipentaerythritol-hexakis(3-mercaptopropionate) (DiPETMP).

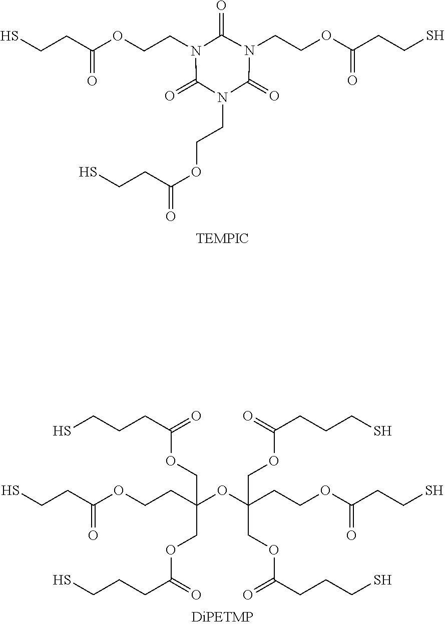 [SMILES: O=C(CCS)OCCn1c(=O)n(CCOC(=O)CCS)c(=O)n(CCOC(=O)CCS)c1=O ; O=C(CCS)OCCC(COC(=O)CCCS)(COC(=O)CCCS)OC(CCOC(=O)CCS)(COC(=O)CCCS)COC(=O)CCCS]

The detailed compositions of the individual formulations are shown in the overleaf Table 4.

TABLE 4
First (PF) and second (MF) components of two-component compositions according
to the state of the art and in inventive uses with varying thiols

| | | BAPA^{Phn} | Phn-6C-bisNorb | TATATO | TEMPMP | TEMPIC | DiPETMP | Ivocerin |
|---|---|---|---|---|---|---|---|---|
| Example | Formulation | [wt %] | (E5) [wt %] | [wt %] | [wt %] | [wt %] | [wt %] | [wt %] |
| Comparative Example 1 (C1) | PF1 | 15 | — | 12.5 | 71.0 | — | — | 1.5 |
| Comparative Example 6 (C6) | PF2 | 15 | — | 12.5 | | 71.0 | — | 1.5 |
| Comparative Example 7 (C7) | PF3 | 15 | — | 12.5 | | — | 71.0 | 1.5 |
| Example 10 (E10) | PF1 | — | 15 | 12.5 | 71.0 | — | — | 1.5 |
| Example 15 (E15) | PF2 | — | 15 | 12.5 | | 71.0 | — | 1.5 |
| Example 16 (E16) | PF3 | — | 15 | 12.5 | | — | 71.0 | 1.5 |
| C1 and E10 | MF1 | — | — | 32.0 | 67.0 | — | — | 1.0 |
| C6 and E15 | MF2 | — | — | 32.0 | — | 67.0 | — | 1.0 |
| C7 and E16 | MF3 | — | — | 32.0 | — | — | 67.0 | 1.0 |

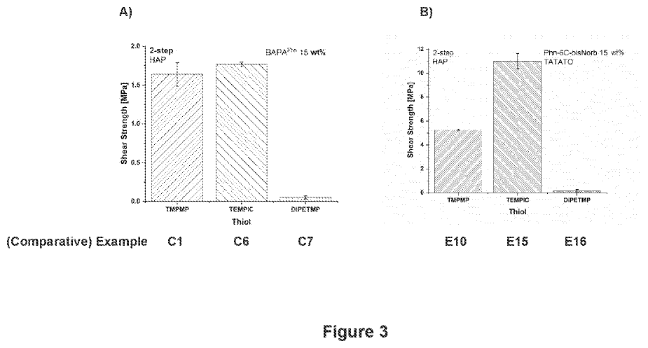

The four new two-component compositions of Comparative Examples 6 and 7 as well as Examples 15 and 16 were applied on hydroxyapatite surfaces in the same way as above, (pre)cured, and the shear bond strength of the composition was examined. The results obtained are, together with the results previously obtained with the TMPMP-containing compositions of Comparative Example 1 and Example 1, shown in FIGS. 3A and 3B.

In FIG. 3 it is immediately noticeably that using DiPETMP as the thiol, according to the state of the art as well as in the inventive use, only very low shear bond strengths were achieved. Without wishing to be bound by theory, the inventors assume that this is, on the one hand, due to the high (or too high) degree of crosslinking achieved with the hexavalent thiol, so that only insufficient amounts of the phosphonic acid groups serving as adhesion motifs of the primers can contact the substrate surface and stay in contact with it. It is therefore to be assumed that with the use of strongly cross-linking thiols, such as DiPETMP, the use of a primer with a larger spacer chain length and/or a larger number of adhesion motifs AM will lead to better results.

However, while the replacement of the thiol of TMPMP with TEMPIC in experiments with BAPA^{Phn} as the primer resulted in only a slight improvement of the values by approximately 5%, with the inventive use of the primer Phn-6C-bisNorb (E5) in Example 15, this replacement resulted in a more than 2 times higher shear bond strength compared to Example 1 (11.00±0.64 MPa vs. 5.25±0.08 MPa). Without wishing to be bound by this, the inventors attribute that, similar to the comparison between TATATO and GDVA as monomers, to the higher rigidity of TEMPIC compared to TMPMP.

Subsequently, a series of further comparative examples and examples for two-component compositions as combinations of TATATO as the monomer, TEMPIC as the thiol and Ivocerin as the initiator were prepared with the primers listed in the overleaf Tables 5 and 6.

TABLE 5
First (PF) and second (MF) components of two-component compositions according to the state of the
art and in inventive uses with varying primers in the primer formulation PF as the first component

| | | Primer | TATATO | TEMPIC | Ivocerin | SBS HAP |
|---|---|---|---|---|---|---|
| Example | Formulation | 15 wt % | [wt %] | [wt %] | [wt %] | [MPa] |
| Comparative Example 6 (C6) | PF | BAPA^{Phn} (S1) | 12.5 | 71.0 | 1.5 | 1.8 |
| Comparative Example 8 (C8) | PF | BAPA^{bisPhn} (S2) * | 11.5 | 70.0 | 1.5 | 3.9 |
| Comparative Example 9 (C9) | PF | Phn-1C-Allo (S3) | 12.5 | 71.0 | 1.5 | 3.1 |
| Comparative Example 10 (C10) | PF | Phn-6C-Allo (S4) | 12.5 | 71.0 | 1.5 | 3.5 |
| Comparative Example 11 (C11) | PF | Phn-6C-bisAllo (S5) | 12.5 | 71.0 | 1.5 | 5.2 |
| Comparative Example 12 (C12) | PF | Phn-1C-Allcarb (S6) | 12.5 | 71.0 | 1.5 | 2.7 |
| Comparative Example 13 (C13) | PF | Phn-6C-Allcarb (S7) | 12.5 | 71.0 | 1.5 | 3.1 |
| Comparative Example 14 (C14) | PF | Phn-6C-bisAllcarb (S8) | 12.5 | 71.0 | 1.5 | 4.1 |
| Comparative Example 15 (C15) | PF | Phn-6C-Enyl (S9) * | 11.5 | 70.0 | 1.5 | 2.9 |
| Comparative Example 16 (016) | PF | Phn-11C-Enyl (S10) * | 11.5 | 70.0 | 1.5 | 4.1 |
| Example 15 (E15) | PF | Phn-6C-bisNorb (E5) | 12.5 | 71.0 | 1.5 | 11.0 |
| Example 17 (E17) | PF | Alendronate-Norb (E1) * | 11.5 | 70.0 | 1.5 | 3.7 |
| Example 18 (E18) | PF | Phn-1C-Norb (E3) | 12.5 | 71.0 | 1.5 | 3.0 |
| Example 19 (E19) | PF | Phn-6C-Norb (E4) | 12.5 | 71.0 | 1.5 | 3.8 |
| Example 20 (E20) | PF | bisPhn-6C-bisNorb (E6) * | 12.5 | 71.0 | 1.5 | 6.8 |
| All foregoing examples | MF | — | 32.0 | 67.0 | 1.0 | |

* For improving solubility in the formulation, 1 wt % of water and 1 wt % of ethanol were added at the expense of the amount of monomer and thiol.

TABLE 6
First (PF) and second (MF) components of two-component compositions according to the state of the
art and in inventive uses with varying primers in the primer formulation PF as the first component

| | | Primer | TATATO | TEMPIC | Ivocerin | SBS TIO₂ |
|---|---|---|---|---|---|---|
| Example | Formulation | 15 wt % | [wt %] | [wt %] | [wt %] | [MPa] |
| Comparative Example 6 (C6) | PF | BAPA^{Phn} (S1) | 12.5 | 71.0 | 1.5 | 3.4 |
| Comparative Example 8 (C8) | PF | BAPA^{bisPhn} (S2) * | 11.5 | 70.0 | 1.5 | 4.8 |
| Comparative Example 9 (C9) | PF | Phn-1C-Allo (S3) | 12.5 | 71.0 | 1.5 | 1.7 |
| Comparative Example 10 (C10) | PF | Phn-6C-Allo (S4) | 12.5 | 71.0 | 1.5 | 2.3 |
| Comparative Example 11 (C11) | PF | Phn-6C-bisAllo (S5) | 12.5 | 71.0 | 1.5 | 2.3 |
| Comparative Example 12 (C12) | PF | Phn-1C-Allcarb (S6) | 12.5 | 71.0 | 1.5 | 1.8 |
| Comparative Example 13 (C13) | PF | Phn-6C-Allcarb (S7) | 12.5 | 71.0 | 1.5 | 2.2 |
| Comparative Example 14 (C14) | PF | Phn-6C-bisAllcarb (S8) | 12.5 | 71.0 | 1.5 | 2.4 |
| Comparative Example 15 (C15) | PF | Phn-6C-Enyl (S9) * | 11.5 | 70.0 | 1.5 | 2.0 |
| Comparative Example 16 C16) | PF | Phn-11C-Enyl (S10) * | 11.5 | 70.0 | 1.5 | 2.3 |
| Example 15 (E15) | PF | Phn-6C-bisNorb (E5) | 12.5 | 71.0 | 1.5 | 7.9 |
| Example 17 (E17) | PF | Alendronate-Norb (E1) * | 11.5 | 70.0 | 1.5 | 7.5 |
| Example 18 (E18) | PF | Phn-1C-Norb (E3) | 12.5 | 71.0 | 1.5 | 2.8 |
| Example 19 (E19) | PF | Phn-6C-Norb (E4) | 12.5 | 71.0 | 1.5 | 2.7 |
| Example 20 (E20) | PF | bisPhn-6C-bisNorb (E6) | 12.5 | 71.0 | 1.5 | 4.5 |
| All foregoing examples | MF | — | 32.0 | 67.0 | 1.0 | |

* For improving solubility in the formulation, 1 wt % of water and 1 wt % of ethanol were added at the expense of the amount of monomer and thiol.

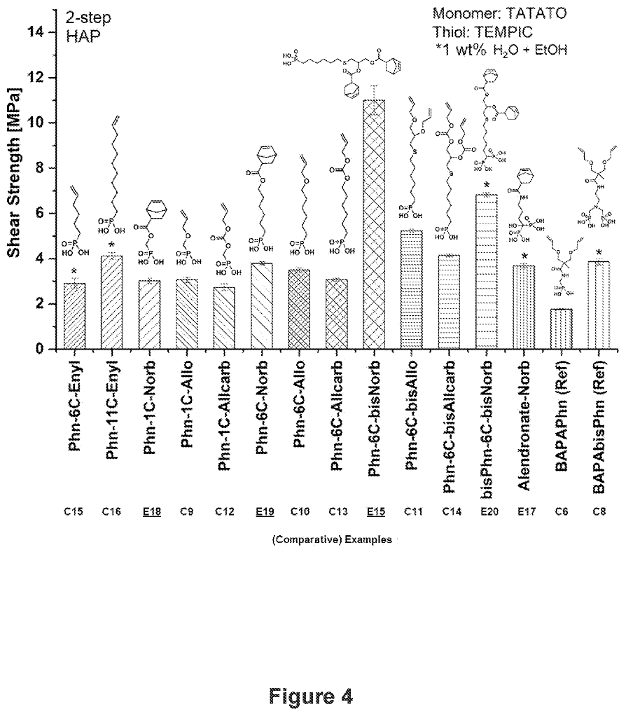

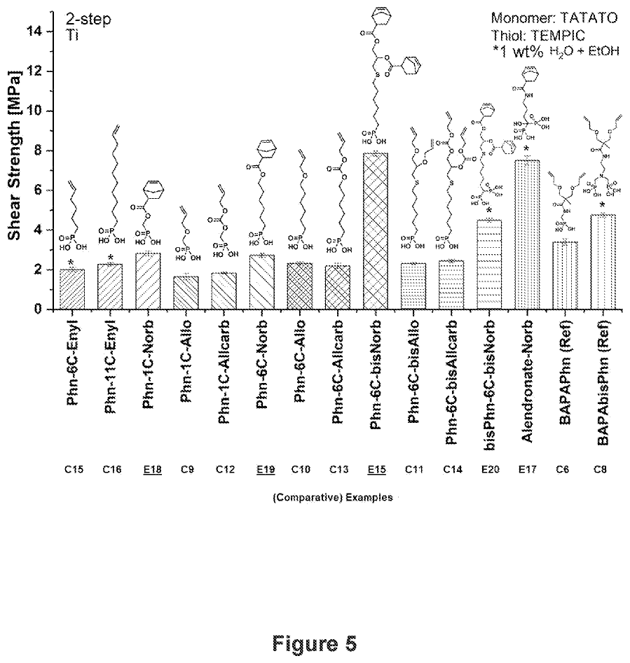

These new two-component compositions of Comparative Examples 8 to 16 and Examples 17 to 20 were applied to hydroxyapatite surfaces in the same way as above, but now also—just like those of Comparative Example 6 and Example 15—to titanium oxide surfaces, (pre-)cured, and the shear bond strength (SBS) of the individual coatings was examined. The results obtained are, together with the results previously obtained for Comparative Example 6 and Example 15, shown in Table 5 and FIG. 4 for hydroxyapatite (“HAP”) and in Table 6 and FIG. 5 for titanium dioxide (“Ti”).

From Table 5 and FIG. 4 it may be seen that primers with a chain length of the spacer between the polymerizable groups PG (vinyl, allyl or norbornenyl) and the adhesion motifs AM (here phosphonic acid groups throughout) with at least 6 carbon or hetero atoms on hydroxyapatite tend to result in better shear bond strengths than the shorter-chain spacers in Comparative Examples 15, 9, 12, and 6 and in Example 18 (Phn-1C-Norb). In addition, it was shown that primers with more than one PG and/or more than one AM also tend to show better results than those with only one corresponding moiety (i.e., in FIG. 4 all results left of the peak value for Example 15).

Furthermore, it is noticeable that the reference primer BAPA^{Phn} from Comparative Example 6 showed—despite the presence of two allyl groups as PG and a spacer length of 6 atoms between the phosphonic acid moiety and each of the two allyl groups —by far the worst result. In contrast, the measurements of Comparative Examples 11, 14 and 8, i.e., primers with 9 to 11 atoms between PG and AM, showed three times higher shear bond strengths, with the first two also containing only one AM and two allyl groups as PG, while BAPA^{bisPhen} has an additional phosphonic acid moiety (HO)₂P(═O)—.

Nevertheless, in a direct comparison, the inventively used primers with norbornenyl as PG also showed better results than the three best primers according to the state of the art: Alendronate-Norb from Example 17 with only one PG and two AM showed approximately the same effectiveness as BAPA^{bisPhn} with two PG and two AM and was hardly worse than Phn-6C-bisAllcarb with one AM and two allyl groups as PG. And Phn-6C-bisNorb from Example 15 as well as bisPhn-6C-bisNorb from Example 20 with two norbornenyl PG and one or two phosphonic acid moieties each resulted in clearly the best shear bond strengths SBS of the cured coatings on hydroxyapatite, while Phn6C-bisNorb from Example 15 with only one AM surprisingly showed even better results. Apparently, the inventors had from the very beginning coincidentally chosen the best of the new primers for their examinations.

With the experiments on titanium oxide surfaces, the situation was different. As can be seen in Table 6 and FIG. 5, again longer-chained spacers with at least 6 atoms between PG and AM moieties are to be preferred, however, in these cases, the two BAPA-containing primers of Comparative Examples 6 and 8 showed the best results of all primers according to the state of the art, with BAPA^{bisPhn} having two AM and two PG again showing better shear bond strengths than BAPA^{Phn} having only one phosphonic acid moiety. The primer bisPhn-6C-bisNorb from Example 20 with also two AM and two norbornenyl PG used according to the invention, however, is almost on a par with BAPA^{bisPhn}, while Alendronate-Norb from Example 17 with only one Norb group and two AM, which had shown similar results as BAPA^{bisPhn} on hydroxyapatite before, was now clearly superior and only superseded by Phn-6C-bisNorb with two norbornenyl PG and only one phosphonic acid AM from Example 15, which thus showed the best values.

However, the primers of Examples 18 and 19 with only one norbornenyl PG and one phosphonic acid moiety each showed better results on titanium oxide than all primers according to the state of the art, except for the two BAPA-containing ones—and thus even better than the primers of Comparative Examples 11 and 14 with two allyl PG each; even though Phn-1C-Norb from Example 18 comprised a spacer with a chain length of only 3 atoms.

In summary, it can be stated that the inventive combinations of 5-norbornen-2-yl groups as polymerizable groups PG and phosphonic acid groups (HO)₂P(═O)— as adhesion motifs AM in primers for polymerizable thiol-ene-based two-component compositions are clearly superior to those according to the state of the art. Apparently, the reactivity of the norbornenyl groups during polymerization of the primer formulation and the quick integration of the primer into the monomer matrix resulting therefrom are more prevalent here than the adhesive effect on the coated surface caused by the adhesion motif.

Subsequently, further two-component compositions were prepared using the previously prepared primers with pyrocatechol (or catechol) groups as adhesion motifs AM and examined with regard to the shear bond strength (SBS) of the coatings obtained therefrom in the same way as above, with the values for the BAPA-containing primers of Comparative Examples 6 and 8 and for the best primer Phn-6C-bisNorb from Example 15 used according to the invention also being listed for comparative purposes. The new pyrocatechol-containing primer compositions used were again combinations of TATATO as the monomer. TEMPIC as the thiol, and Ivocerin as the initiator, now prepared with the primers listed in the following Tables 7 and 8, with Comparative Example 18 being a literature value according to Olofsson et al., v.s. However, this value was not determined on hydroxyapatite, but on bones, namely with a two-component composition also containing TATATO as the monomer and TEMPIC as the thiol, but the initiator was not Ivocerin, but camphorquinone. The primer used was a 1:1 mixture of DOPA-Allyl and DOPA-Thiol:

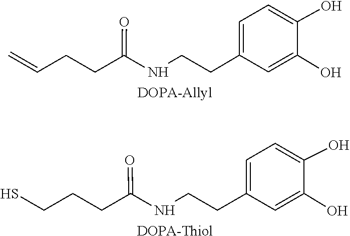

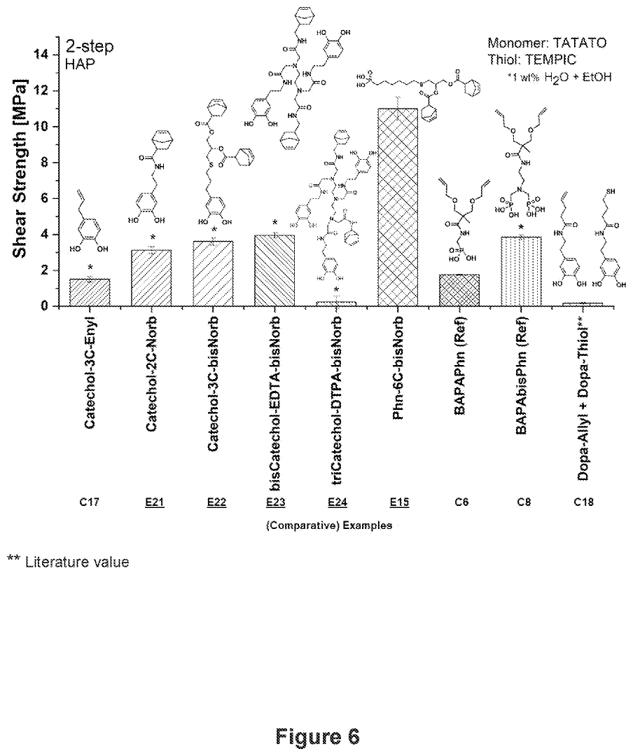

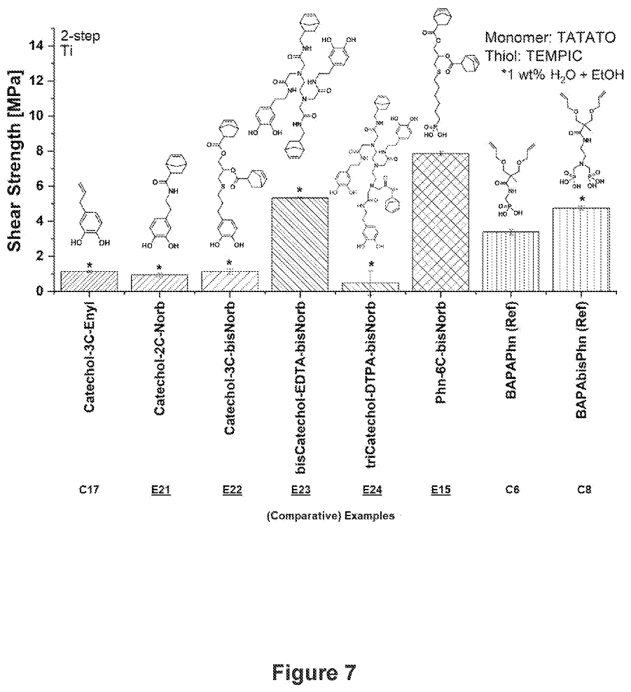

The results are also shown in Tables 7 and 8 and in FIGS. 6 and 7, while Example 21 with the primer catechol-2C-Norb (E2) when used on TiO₂ surfaces (Table 8 and FIG. 7) known from the reference cited above, “Ye et al.”, is to be seen as a comparative example. On the HAP surface (Table 7 and FIG. 6), which is to be seen as a model surface for the use as a bone adhesive, however, it is to be regarded an inventive example.

TABLE 7
First (PF) and second (MF) components of two-component compositions according to the state of the
art and in inventive uses with varying primers in the primer formulation PF as the first component

| | | Primer | TATATO | TEMPIC | Ivocerin | SBS HAP |
|---|---|---|---|---|---|---|
| Example | Formulation | 15 wt % | [wt %] | [wt %] | [wt %] | [MPa] |
| Comparative Example 6 (C6) | PF | BAPA^{Phn} (S1) | 12.5 | 71.0 | 1.5 | 1.8 |
| Comparative Example 8 (C8) | PF | BAPA^{bisPhn} (S2) * | 11.5 | 70.0 | 1.5 | 3.9 |
| Comparative Example 17 (C17) | PF | Catechol-3C-Enyl (S11) * | 11.5 | 70.0 | 1.5 | 1.5 |
| Comparative Example 18 (C18) | PF | DOPA-Allyl + DOPA-Thiol ** | — | — | — | 0.29 |
| Example 15 (E15) | PF | Phn-6C-bisNorb (E5) | 12.5 | 71.0 | 1.5 | 11.0 |
| Example 21 (E21) | PF | Catechol-2C-Norb (E2) * | 11.5 | 70.0 | 1.5 | 3.1 |
| Example 22 (E22) | PF | Catechol-3C-bisNorb (E7) * | 11.5 | 70.0 | 1.5 | 3.6 |
| Example 23 (E23) | PF | bisCatechol-EDTA-bisNorb (E8) * | 11.5 | 70.0 | 1.5 | 4.0 |
| Example 24 (e24) | PF | triCatechol-DTPA-bisNorb (E9) * | 11.5 | 70.0 | 1.5 | 0.4 |
| Foregoing examples except C18 | MF | — | 32.0 | 67.0 | 1.0 | |
| Comparative Example 18 (C18) | MF | — | 32.1 | 67.8 | 0.1 *** | |

* For improving solubility in the formulation, 1 wt % of water and 1 wt % of ethanol were added at the expense of the amount of monomer and thiol.
** SBS value from literature; Olofsson et al. (v.s.)
*** Camphorquinone as the initiator

TABLE 8
First (PF) and second (MF) components of two-component compositions according to the state of the
art and in inventive uses with varying primers in the primer formulation PF as the first component

| | | Primer | TATATO | TEMPIC | Ivocerin | SBS TIO₂ |
|---|---|---|---|---|---|---|
| Example | Formulation | 15 wt % | [wt %] | [wt %] | [wt %] | [MPa] |
| Comparative Example 6 (C6) | PF | BAPA^{Phn} (S1) | 12.5 | 71.0 | 1.5 | 3.4 |
| Comparative Example 8 (C8) | PF | BAPA^{bisPhn} (S2) * | 11.5 | 70.0 | 1.5 | 4.8 |
| Comparative Example 17 (C17) | PF | Catechol-3C-Enyl (S11) * | 11.5 | 70.0 | 1.5 | 1.1 |
| Example 15 (E15) | PF | Phn-6C-bisNorb (E5) | 12.5 | 71.0 | 1.5 | 7.9 |
| Example 21 (E21); here a | PF | Catechol-2C-Norb (E2) * | 11.5 | 70.0 | 1.5 | 0.9 |
| comparative example | | | | | | |
| Example 22 (E22) | PF | Catechol-3C-bisNorb (E7) * | 11.5 | 70.0 | 1.5 | 1.1 |
| Example 23 (E23) | PF | bisCatechol-EDTA-bisNorb (ES) * | 11.5 | 70.0 | 1.5 | 5.3 |
| Example 24 (E24) | PF | triCatechol-DTPA-bisNorb (E9) * | 11.5 | 70.0 | 1.5 | 0.5 |
| All foregoing examples | MF | — | 32.0 | 67.0 | 1.0 | |

* For improving solubility in the formulation, 1 wt % of water and 1 wt % of ethanol were added at the expense of the amount of monomer and thiol.

From the SBS results in Table 7 and FIG. 6 it is noticeable that the inventively used primer triCatechol-DTPA-bisNorb from Example 24 had resulted in a shear bond strength that was only slightly better than the value cited in literature for the mixture of DOPA-Allyl and DOPA-Thiol, but significantly worse than the other values for pyrocatechol-containing primers. However, this is partly due to the fact that these are weight-related primer portions in the formulations, as well as the fact that the three inventively used primers of Examples 21 to 23 did not show better results than the extremely short-chained primer from Comparative Example 17 with a spacer length of only one single carbon atom. Taking into account the highly different molecular weights, the primer formulations of Examples 21 to 24 contained only about half (E21), one third (E22), one fifth (E23), or one seventh (E24) of the molecular amount of primer in Comparative Example 17 (Catechol-3C-Enyl). However, the comparatively bad value of Example 24 is, according to the opinion of the inventors, probably due to the fact that this triCatechol-DTPA-bisNorb primer was not completely soluble in the monomer/thiol mixture, despite the addition of water and ethanol. Further tests with this primer are, therefore, the subject of further investigations by the inventors.

The effectiveness of the three inventively used primers of Examples 21 to 23 or 22 and 23 is, however, to be estimated much higher than reflected by the values in Table 8 and the graph in FIG. 6 because of the high differences in molecular weight compared to those of Comparative Example 17, but also to the two BAPA-containing primers of Comparative Examples 6 and 8. Of course, this also applies to the above examples with phosphonic acid moieties as adhesion motifs.

In a direct comparison between the primers with two polymerizable groups PG and one adhesion motif AM, i.e., Catechol-3C-bisNorb (E22) and BAPA^{Phn} (C6), the inventively used primer achieved a shear bond strength value twice as high. In a comparison between the primers with two PG and two AM each, i.e., bisCatechol-EDTA-bisNorb (from E23) and BAPA^{bisPhn} (from C8), the inventively used primer only shows slightly better results, however, this is one the one hand again due to its higher molecular weight and on the other hand to the fact that phosphonic acid groups on hydroxyapatite surfaces are more effective adhesion motifs than pyrocatechyl residues, as is clearly shown by the peak value of the primer from Example 15 shown here again for comparative purposes. It is approximately three times higher than that of Example 22, even though both primers are structured analogously (2 PG and 1 AM each) and have comparable molecular weights. In particular, this last effect has a stronger impact on titanium oxide surfaces, as is shown by the values in Table 8 and the graph in FIG. 7. Here, the peak value of pyrocatechol the inventively used primer from Example 15 is even approximately seven times higher than that from Example 22, which means that phosphonic acid groups are to be preferred as AM especially on titanium oxide. Without wishing to be bound by this, the inventors assume that this might partly be due to a certain inhibiting effect of the phenolic OH groups during radical polymerization.

In addition to the, again due to the solubility, bad value for the triCatechol-DTPA-bisNorb primer from Example 24, it is noticeable in this case that the inventively used primer from Example 23 with two norbonenyl PG and two Pyrocatechyl AM shows better results here compared to the peak value of Example 14 than before on hydroxyapatite, which again proves the inventive preference of at least two norbornenyl groups as PG and at least two phosphonic acid or pyrocatechyl groups as AM. Also, the value for Example 23 is in this case even 10% better than that for the BAPA primer with an analogous structure from Comparative Example 8—even though the latter has two phosphonic acid PG that provide stronger adhesion than the two pyrocatechyl groups of bisCatechol-EDTA-bisNorb from Example 23.

### Examples 25 to 36, Comparative Examples 19 to 30—One-Component Compositions

In analogy to above, this group of examples comprised the preparation of one-component compositions by mixing monomers, thiols, initator, and one primer used according to the invention or according to the state of the art each, applying it to a polished substrate surface made of hydroxyapatite or titanium dioxide, irradiated for 20 s and thus cured, placed in modified simulated body liquid, stored for 24 h at 37° C., and then tested with regard to the shear bond strength (SBS) of the cured coating using the VTSYIQI SF-30 digital force gauge with a V-shaped test rod.

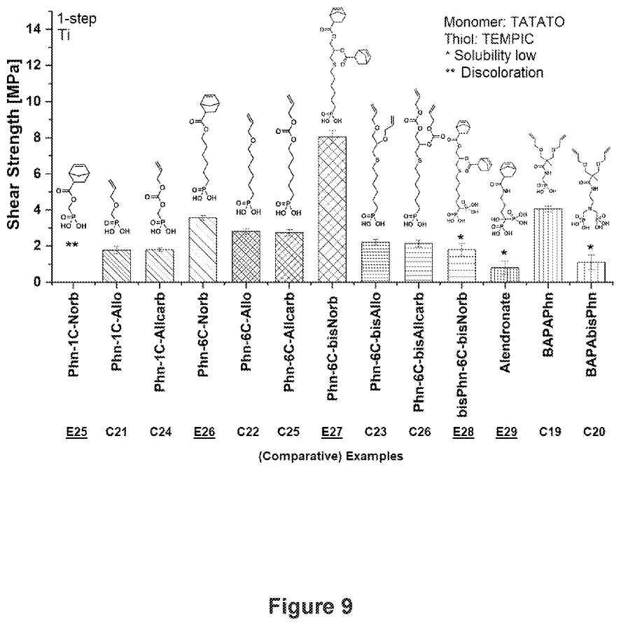

All formulations of the one-component compositions contained a monomer TATATO (27.0 wt %), a thiol TEMPIC (57.0 wt %) and Ivocerin (1.0 wt %) as the initiator. The primers used were exclusively phosphonic acid groups as AM-containing compounds (15 wt %). Table 9 shows the formulations thus prepared, which again each contained additionally 0.02 wt % of pyrogallol as an oxidation stabilizer, as well as the shear bond strength values on hydroxyapatite (HAP) and titanium dioxide (TiO₂) surfaces thus obtained, these values being also graphically shown in FIG. 8 and FIG. 9, respectively.

TABLE 9
One-component compositions according to the state of the art and
according to the present invention with varying primers

| | | SBS HAP | SBS TiO₂ |
|---|---|---|---|
| Example | Primer | [MPa] | [MPa] |
| Comparative | BAPA^{Phn} (S1) | 8.1 | 4.1 |
| Example 19 (C19) | | | |
| Comparative | BAPA^{bisPhn} (S2) * | 1.0 | 1.1 |
| Example 20 (C20) | | | |
| Comparative | Phn-1C-Allo (S3) | 1.6 | 1.8 |
| Example 21 (C21) | | | |
| Comparative | Phn-6C-Allo (S4) | 3.6 | 2.8 |
| Example 22 (C22) | | | |
| Comparative | Phn-6C-bisAllo (S5) | 3.5 | 2.2 |
| Example 23 (C23) | | | |
| Comparative | Phn-1C-Allcarb (S6) | 1.8 | 1.8 |
| Example 24 (C24) | | | |
| Comparative | Phn-6C-Allcarb (S7) | 3.6 | 2.7 |
| Example 25 (C25) | | | |
| Comparative | Phn-6C-bisAllcarb (S8) | 3.5 | 2.1 |
| Example 26 (C26) | | | |
| Example 25 (E25) | Phn-1C-Norb (E3) ** | 0.0 | 0.0 |
| Example 26 (E26) | Phn-6C-Norb (E4) | 8.0 | 3.6 |
| Example 27 (E27) | Phn-6C-bisNorb (E5) | 15.4 | 8.0 |
| Example 28 (E28) | bisPhn-6C-bisNorb (E6) * | 0.5 | 1.8 |
| Example 29 (E29) | Alendronate-Norb (E1) * | 1.1 | 0.8 |

*: not completely soluble
**: discoloration

The primers marked with * were not completely soluble in the formulations, and the primer marked with ** from Example 25 led to a strong brown discoloration of the formulation, which greatly hindered the penetration of the radiation during exposure and significantly reduced the curing depth, so that almost no adhesion to the substrate was noticeable. Further tests with these primers, also with the addition of H₂O and ethanol again to improve solubility, are therefore subject of current investigations conducted by the inventors.

As can be seen from Table 9 and FIG. 8, however, the other two inventively used primers of Examples 26 and 27, which did not show any problems, were again superior to those of the comparative examples, of which BAPA^{Phn} (S1) from Comparative Example 19 showed by far the best results because it had achieved more than twice the shear bond strength of all other comparative primers. Phn-6C-Norb (E4) from Example 26 on hydroxyapatite (HAP) still resulted in almost the same shear bond strength value, even though this new primer contains only one polymerizable norbornenyl group, while BAPA^{Phn} (S1) has two allyl groups. The best primer according to the invention was, as expected, again Phn-6C-bisNorb (E5) from Example 27 with an analogous structure to BAPA^{Phn} (S1) and two norbornenyl groups and a phosphonic acid group as the adhesion motif, which even reached a shear bond strength value almost twice as high as that of BAPA^{Phn} (S1)—on hydroxyapatite as well as on titanium dioxide, as can be seen in FIG. 9. Also on TiO₂, the primer from Example 26 with only one polymerizable group showed better results than all comparative primers except BAPA^{Phn} (S1), the value of which was approximately 10% higher here.

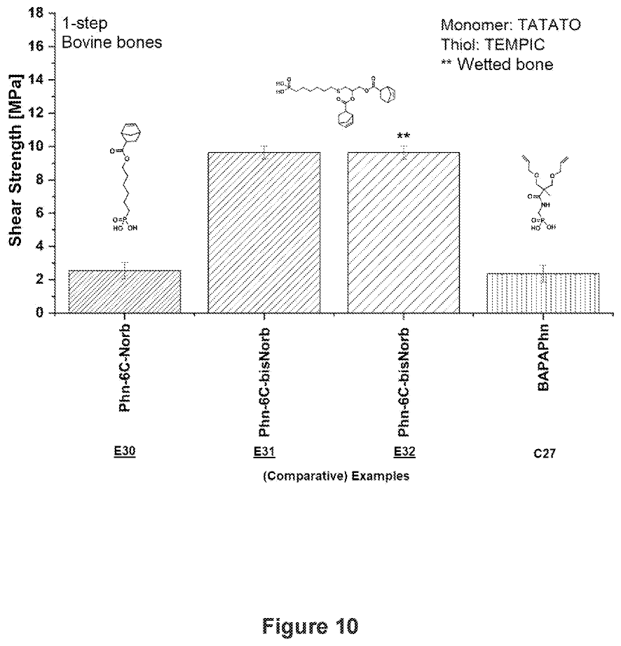

Subsequently, the three primers of BAPA^{Phn} (S1), Phn-6C-Norb (E4), and Phn-6C-bisNorb (E5) were also tested on the bovine toe bone discs prepared as described above in the form of analogous one-component compositions that, in addition to 15 wt % of primer, again contained 27.0 wt % of TATATO as the monomer, 57.0 wt % of TEMPIC as the thiol, and 1.0 wt % of Ivocerin as the initiator. Application, curing, and measuring were conducted as above. Furthermore, the best inventively used primer, Phn-6C-bisNorb (E5), was tested for a second time on such a bone disc, wherein, however, the disc was wetted before application by rubbing in distilled water with a micro brush for 20 s and then letting it air-dry for 10 s. The composition of the formulations and the results obtained therewith are shown in the following Table 10, and the latter are also graphically shown in FIG. 10.

TABLE 10
One-component composition according to the state of the art and
according to the present invention on bovine bones

| | | SBS Bones |
|---|---|---|
| Example | Primer | [MPa] |
| Comparative | BAPA^{Phn} (S1) | 2.4 |
| Example 27 (C27) | | |
| Example 30 (E30) | Phn-6C-Norb (E4) | 2.5 |
| Example 31 (E31) | Phn-6C-bisNorb (E5) | 9.6 |
| Example 32 (E32) | Phn-6C-bisNorb (E5) ** | 9.6 |

**: wetted bones

The ratio of the measured values was, as expected, similar to the previous ones on hydroxyapatite, which is used as a model substance for experiments with bone adhesives, even though the shear strength on smoothly polished, homogeneous hydroxyapatite surfaces was comprehensibly higher than on bone discs. However, the two inventively used primers on bone material exceeded the known primer BAPA^{Phn} (S1) from Comparative Example 27 even more clearly than before. In this case, the shear bond strength value achieved with Phn-6C-Norb (E4) in Example 30 was even slightly higher than the comparative value for the primer with two polymerizable groups, however, that for the primer Phn-6C-bisNorb (E5) with an analogous structure was even almost five times as high as that of the comparison—on dry (Example 31) as well as on pre-wetted and shortly dried bovine bones (Example 32).

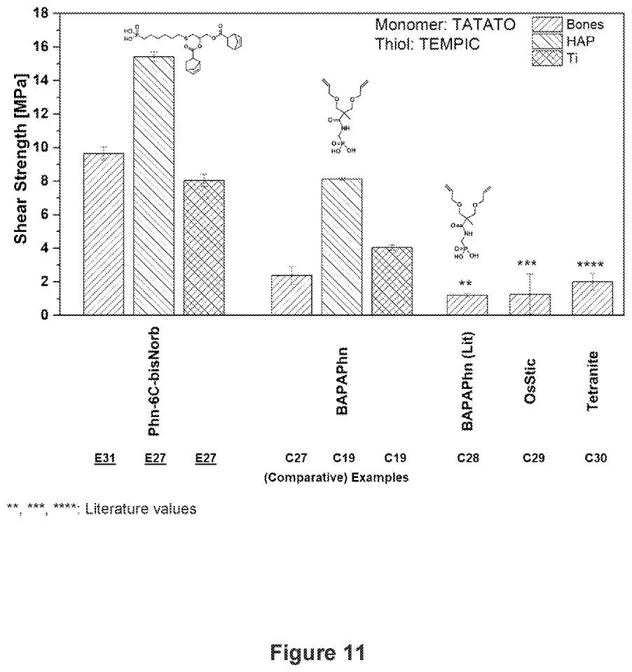

The following Table 11 again shows the results for the best inventively used primer, Phn-6C-bisNorb (E5), and the comparative primer BAPA^{Phn} (S1) with an analogous structure on all three tested surfaces, bovine bones (“bones”), hydroxyapatite (HAP), and titanium dioxide (“Ti”), additionally including, as comparative examples, literature values for BAPA^{Phn} (from Granskog et al., v.s.) as well as the bone adhesives and cements mentioned at the beginning and available under the trademarks OsStic™ by PBC Biomed and Tetranite™ by LaunchPad Medical (from Pujari-Palmer et al., v.s.; and Kirillova et al., v.s., respectively), each on hydroxyapatite surfaces. FIG. 11 is a graphic representation of these values.

TABLE 11
One-component compositions according to the state of the art and used
according to the invention

| | | SBS B | SBS HAP | SBS TiO₂ |
|---|---|---|---|---|
| Example | Primer | [MPa] | [MPa] | [MPa] |
| Comparative | BAPA^{Phn} (S1) | 2.4 | | |
| Example 27 (C27) | | | | |
| Comparative | BAPA^{Phn} (S1) | | 8.1 | 4.1 |
| Example 19 (C19) | | | | |
| Comparative | BAPA^{Phn} ** | 1.2 | | |
| Example 28 (C28) | | | | |
| Comparative | OsStic ™ *** | 1.5 | | |
| Example 29 (C29) | | | | |
| Comparative | Tetranite™ **** | 1.9 | | 3.3 |
| Example 30 (C30) | | | | |
| Example 31 (E31) | Phn-6C-bisNorb (E5) | 9.6 | | |
| Example 27 (E27) | Phn-6C-bisNorb (E5) | | 15.4 | 8.0 |

**: Granskog et al. (v.s.)
***: Pujari-Palmer et al. (v.s.)
****: Kirillova et al. (v.s.)

One-component compositions with the best primer according to the present invention, Phn-6C-bisNorb (E5), thus show significantly better results than previously known systems on all tested surfaces. Due to the generally higher shear strength on hydroxyapatite surfaces than on bone discs, as mentioned above, significantly lower values than those given in the literature for hydroxyapatite are to be expected for the known systems from Comparative Examples 28 to 30 on bovine bones, but probably also on titanium dioxide surfaces.

Finally, the inventors also conducted experiments with filled one-component compositions using Phn-6C-bisNorb (E5), wherein the formulation again containing 15 wt % of the primer, 27.0 wt % of TATATO, 57.0 wt % of TEMPIC, and 1.0 wt % of Ivocerin was mixed with different amounts of hydroxyapatite or titanium dioxide as a filler.

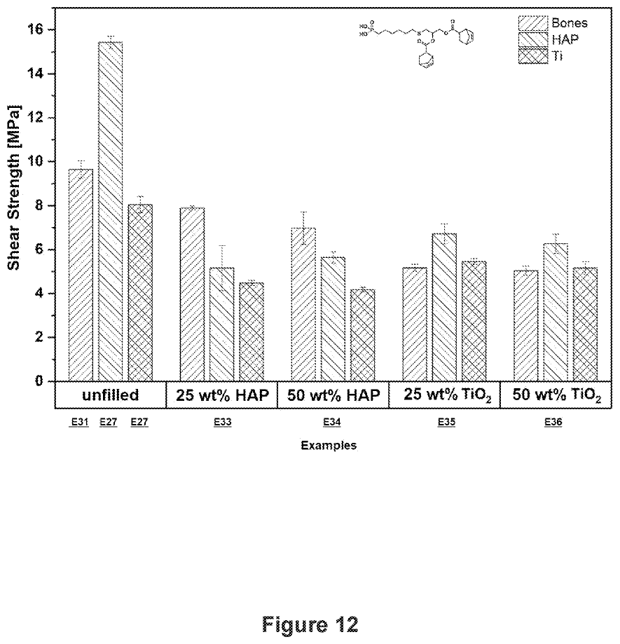

In order to avoid problems with regard to the penetration depth of radiation, the fillers were functionalized with the inventively used bis(phosphonic acid) primer, bisPhn-6C-bisNorb (E6). Here, 4 g of each filler powder were dispersed with 0.5 g of the primer in 10 ml of MeOH:H₂O (1:4 Vol.), stirred for 24 h, filtered, washed with MeOH (3×50 ml), and dried under high vacuum. The fillers thus functionalized were each mixed in the given parts by weight based on 100 parts by weight of the formulation. The respective compositions and the results obtained therewith on the different surfaces (bones, B, hydroxyapatite, HAP, and titanium dioxide, TiO₂) are shown in the following Table 12, wherein the latter are also graphically shown in FIG. 12. Table 12 also again includes the values of the comparative examples with BAPA^{Phn} as the primer without filler.

TABLE 12
Filled one-component compositions used according to the invention with
Phn-6C-bisNorb (E5) as the primer

| | | | | SBS |
|---|---|---|---|---|
| | Filler | SBS B | SBS HAP | TiO₂ |
| Example | (parts by weight) | [MPa] | [MPa] | [MPa] |
| Comparative | — | 2.4 | | |
| Example 27 (C27) | — | | | |
| Comparative | — | | 8.1 | 4.1 |
| Example 19 (C19) | — | | | |
| Example 31 (E31) | — | 9.6 | | |
| Example 27 (E27) | — | | 15.4 | 8.0 |
| Example 33 (E33) | Hydroxyapatite (25) | 7.9 | 5.2 | 4.5 |
| Example 34 (E34) | Hydroxyapatite (50) | 7.0 | 5.6 | 4.2 |
| Example 35 (E35) | Titanium dioxide (25) | 5.2 | 6.7 | 5.5 |
| Example 36 (E36) | Titanium dioxide (50) | 5.0 | 6.3 | 5.2 |

This shows that the shear bond strengths obtained with the filled formulations are reduced compared to the respective unfilled examples, however, they were superior to the values of the unfilled comparative examples with BAPA^{Phn} as the primer on bovine bones and titanium dioxide. Assuming a similar reduction of the shear bond strength of the comparative examples with a corresponding addition of a filler, i.e., by a factor of approximately 2.5 to 3, the inventive examples would almost certainly also supersede those with BAPA^{Phn} as the primer on hydroxyapatite. Corresponding experiments are subject of current investigations by the inventors.

The above examples and comparative examples thus impressively demonstrate the superiority of the primers and of the thiol-ene-based polymerizable compositions containing the same when used according to the present invention compared to the state of the art.

1. A use of compounds comprising, per molecule, at least one polymerizable C—C double bond, at least one adhesion-providing moiety, and one at least bivalent hydrocarbon moiety in between as a spacer as primers in a thiol-ene-based polymerizable composition for improving the adhesion of the composition on a substrate to be coated therewith, wherein the composition further comprises a radical initiator, monomers with at least two polymerizable C—C double bonds per molecule, and polythiols with at least two SH groups per molecule, wherein the primers comprise 5-norbornen-2-yl groups as polymerizable C—C double bonds,
characterized in that
compounds are used as primers in the polymerizable composition that
a) comprise phosphonic acid groups as adhesion-providing moieties; or
b) comprise groups selected from phosphonic acid groups and 3,4-dihydroxyphenyl groups as adhesion-providing moieties, and that the polymerizable composition is used as a bone adhesive.

2. The use according to claim 1, characterized in that the primers each comprise at least two 5-norbornen-2-yl groups and/or at least two adhesion-providing moieties.

3. The use according to claim 1 or 2, characterized in that the spacer moiety of the primers has a chain length of at least 3 or at least 6 carbon atoms.

4. The use according to claim 1, characterized in that the spacer moiety of the primer has a chain length of not more than 12 or not more than 10 carbon atoms.

5. The use according to claim 1, characterized in that the monomers are selected from compounds with at least two allyl or vinyl ester groups.

6. The use according to claim 5, characterized in that the monomers comprise 1,3,3-triallyl-1,3,5-triazin-2,4,6-trione (TATATO).

7. The use according to claim 1, characterized in that the polythiols are selected from compounds with at least three SH groups per molecule.

8. The use according to claim 7, characterized in that the polythiols comprise tris[2-(3-mercaptopropionyloxy)ethyl]isocyanurate (TEMPIC).

9. The use according to claim 1, characterized in that the composition is a one-component composition.

10. The use according to claim 1, characterized in that the composition is a two-component composition, of which only the first component comprises the primers.

11. The use according to claim 1, characterized in that the composition comprises approximately 0.1 wt % to approximately 5 wt % of a radical initiator, approximately 10 wt % to approximately 50 wt % of monomers, approximately 40 wt % to approximately 80 wt % of polythiols, and approximately 5 wt % to approximately 25 wt % of primers, in such amounts that the sum is 100 wt %.

12. The use according to claim 9, characterized in that the composition contains approximately 0.5 wt % to approximately 2 wt % of a radical initiator, approximately 40 wt % to approximately 60 wt % of monomers, approximately 60 wt % to approximately 80 wt % of polythiols, and approximately 10 wt % to approximately 20 wt % of primers, in such amounts that the sum is 100 wt %.

13. The use according to claim 1, characterized in that the radical initiator is a radical photoinitiator and the composition is photopolymerizable.

14. The use according to claim 9, characterized in that the radical initiator is a radical photoinitiator and the composition is photopolymerizable and the composition is appliable to the substrate to be coated in one step and then curable by irradiation.

15. A norbornene derivative of the following formula 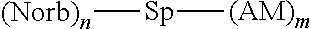
wherein
Norb represents a 5-norbornen-2-yl radical;
each AM independently represents a phosphonic acid group (HO)₂P(═O)— or a 3,4-dihydroxyphenyl groups;
n and m each independently represent an integer ≥1; and
Sp represents a spacer group selected from (n+m)-valent hydrocarbon residues with 1 to 40 carbon atoms;
provided that at least one of n and m represents an integer >1 when AM represents a 3,4-dihydroxyphenyl group.

16. The norbornene derivative according to claim 15, characterized in that
a) one or both of n and m represent(s) 2; and/or
b) S represents an (n+m)-valent hydrocarbon residue with 3 to 20 or 6 to 16 carbon atoms, one or more of which are optionally replaced by a heteroatom selected form O, S and N; and/or
c) AM represents a phosphonic acid group (HO)2P(═O)—.

17. The norbornene derivative according to claim 15,
characterized in that it is selected from the following compounds:
N-(4-hydroxy-4,4-diphosphonobutyl)-5-norbornene-2-carboxylic acid amide sodium salt (N-(4-hydroxy-4,4-diphosphonobutyl)alendronic acid amide sodium salt) (Alendronate-Norb) (E1): 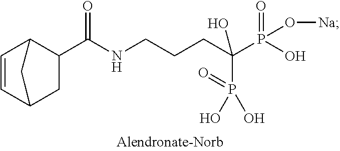
5-norbornen-2-ylcarbonyloxymethylphosphonic acid (Phn-1C-Norb) (E3): 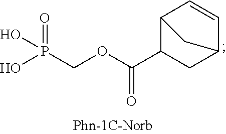 [SMILES: O=C(OCP(=O)(O)O)C1CC2C=CC1C2]
6-(5-norbornen-2-ylcarbonyloxy)hexylphosphonic acid (Phn-6C-Norb) (E4): 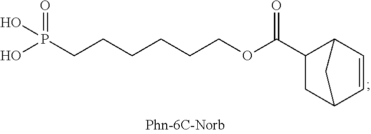 [SMILES: O=C(OCCCCCCP(=O)(O)O)C1CC2C=CC1C2]
6-(2,3-bis(5-norbornen-2-ylcarbonyloxy)propylthio)hexylphosphonic acid (Phn-6C-bisNorb) (E5): 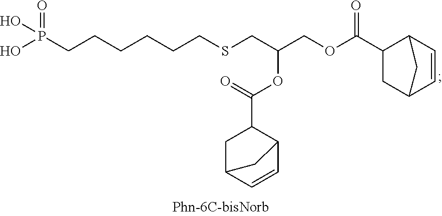 [SMILES: O=C(OCC(CSCCCCCCP(=O)(O)O)OC(=O)C1CC2C=CC1C2)C1CC2C=CC1C2]
6-(2,3-bis(5-norbornen-2-ylcarbonyloxy)propylthio)hexane-1,1-diyl-bis(phosphonic acid) (bisPhn-6C-bisNorb) (E6): 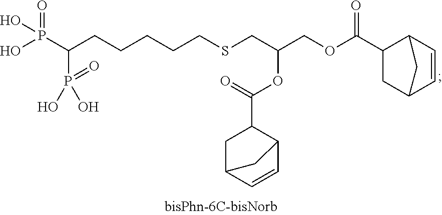 [SMILES: O=C(OCC(CSCCCCCC(P(=O)(O)O)P(=O)(O)O)OC(=O)C1CC2C=CC1C2)C1CC2C=CC1C2]
3-(3-(3,4-dihydroxyphenyl)propylthio)propane-1,2-diyl-bis(norbornen-2-carboxylate) (Catechol-3C-bisNorb) (E7): 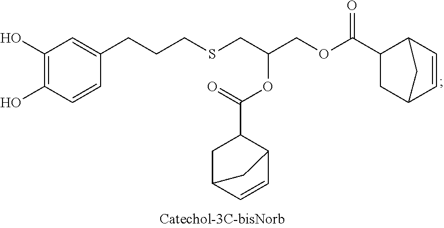 [SMILES: O=C(OCC(CSCCCc1ccc(O)c(O)c1)OC(=O)C1CC2C=CC1C2)C1CC2C=CC1C2]
ethylenediaminetetraacetic acid-bis(2-(3,4-dihydroxyphenyl)ethyl)amide-bis(2-(5-norbornen-2-yl)ethyl)amide (bisCatechol-EDTA-bisNorb) (E8): 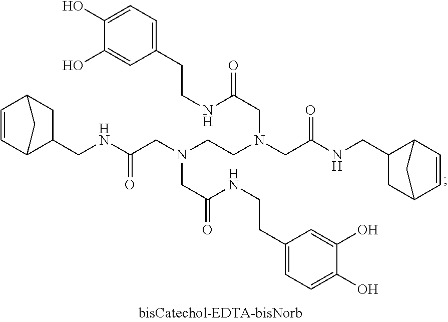 [SMILES: O=C(CN(CCN(CC(=O)NCCc1ccc(O)c(O)c1)CC(=O)NCC1CC2C=CC1C2)CC(=O)NCC1CC2C=CC1C2)NCCc1ccc(O)c(O)c1]
and
diethylenetriaminepentaacetic acid-bis(2-(5-norbornen-2-yl)ethyl)amide-tris(2-(3,4-dihydroxyphenyl)ethyl)amide (triCatechol-DTPA-bisNorb) (E9):

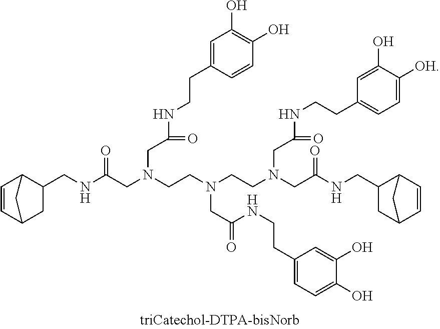 [SMILES: O=C(CN(CCN(CC(=O)NCCc1ccc(O)c(O)c1)CC(=O)NCC1CC2C=CC1C2)CCN(CC(=O)NCCc1ccc(O)c(O)c1)CC(=O)NCC1CC2C=CC1C2)NCCc1ccc(O)c(O)c1]

18. The use according to claim 1, characterized in that the primer used is a norbornene derivative of the following formula 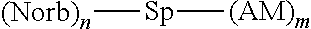
wherein
Norb represents a 5-norbornen-2-yl radical;
each AM independently represents a phosphonic acid group (HO)₂P(═O)— or a 3,4-dihydroxyphenyl groups;
n and m each independently represent an integer ≥1; and
Sp represents a spacer group selected from (n+m)-valent hydrocarbon residues with 1 to 40 carbon atoms;
provided that at least one of n and m represents an integer >1 when AM represents a 3,4-dihydroxyphenyl group.
